(19) United States
(12) Patent Application Publication
(10) Pub. No.: US 2009/0036355 A1
(43) Pub. Date: Feb. 5, 2009

(54) Antisense Modulation of PTP1B Expression

(76) Inventors: Sanjay Bhanot, Carlsbad, CA (US); Brett P. Monia, Encinitas, CA (US); Richard S. Geary, Carlsbad, CA (US); Lise Lunt Kjems, Encinitas, CA (US)

Correspondence Address: McDermott Will & Emery, 4370 La Jolla Village Drive, Suite 700, San Diego, CA, 92122, US

(21) Appl. No.: 11/665,423

(22) PCT Filed: Oct. 13, 2005
(86) PCT No.: PCT/US05/36813

§ 371 Date: Jul. 9, 2008

(30) Foreign Application Priority Data
Oct. 13, 2004 (US) 60618384
Feb. 14, 2005 (US) 60653165
Mar. 24, 2005 (US) 60665555
Jun. 9, 2005 (US) 60688984

(51) Int. Cl. A61K 38/28; A61K 31/711; A61P 3/10; A61P 3/04; A61P 9/10

(52) U.S. Cl. 514/4; 514/44

(57) ABSTRACT

Compositions and methods are provided for decreasing blood glucose levels in an animal or for preventing or de-laying the onset of a rise in blood glucose levels in an animal, comprising administering to said animal an antisense inhibitor of PTP1B expression in combination with at least one glucose-lowering drug. The present invention is also directed to compositions and methods for improving insulin sensitivity in an animal or for preventing or delaying the onset of insulin resistance in an animal. Also provided are compositions and methods for treating or preventing a metabolic condition in an animal. The metabolic condition may be, e.g., diabetes or obesity.

### CROSS REFERENCE TO RELATED APPLICATIONS

This application is related to application No. 60/618,384, filed on Oct. 13, 2004, application No. 60/653,165, filed Feb. 14, 2005, application No. 60/665,555, filed on Mar. 24, 2005, and application No. 60/688,984, filed on Jun. 9, 2005 each of which is herein incorporated by reference in its entirety.

### SEQUENCE LISTING

A paper copy of the sequence listing and a computer-readable form of the sequence listing, on diskette, containing the file named BIOL0044WOSEQ.txt, which was created on Oct. 13, 2005, are herein incorporated by reference.

### BACKGROUND OF THE INVENTION

The process of phosphorylation, defined as the attachment of a phosphate moiety to a biological molecule through the action of enzymes called kinases, represents one course by which intracellular signals are propagated, resulting finally in a cellular response. Within the cell, proteins can be phosphorylated on serine, threonine or tyrosine residues. The extent of phosphorylation is regulated by the opposing action of phosphatases, which remove the phosphate moieties. While the majority of protein phosphorylation within the cell is on serine and threonine residues, tyrosine phosphorylation is modulated to the greatest extent during oncogenic transformation and growth factor stimulation (Zhang, Crit. Rev. Biochem. Mol. Biol., 1998, 33, 1-52).

Because phosphorylation is such a ubiquitous process within cells and because cellular phenotypes are largely influenced by the activity of these pathways, it is currently believed that a number of disease states and/or disorders are a result of either aberrant activation of, or functional mutations in, kinases and phosphatases. Consequently, considerable attention has been devoted recently to the characterization of tyrosine kinases and tyrosine phosphatases.

PTP1B (also known as protein phosphatase 1B and PTPN1) is an endoplasmic reticulum (ER)-associated enzyme originally isolated as the major protein tyrosine phosphatase of the human placenta (Tonks et al., J. Biol. Chem., 1988, 263, 6731-6737; Tonks et al., J. Biol. Chem., 1988, 263, 6722-6730).

An essential regulatory role in signaling mediated by the insulin receptor has been established for PTP1B. PTP1B interacts with and dephosphorylates the activated insulin receptor both in vitro and in intact cells resulting in the downregulation of the signaling pathway (Goldstein et al., Mol. Cell. Biochem., 1998, 182, 91-99; Seely et al., Diabetes, 1996, 45, 1379-1385). In addition, PTP1B modulates the mitogenic actions of insulin (Goldstein et al., Mol. Cell. Biochem., 1998, 182, 91-99). In rat adipose cells overexpressing PTP1B, the translocation of the GLUT4 glucose transporter was inhibited, implicating PTP1B as a negative regulator of glucose transport as well (Chen et al., J. Biol. Chem., 1997, 272, 8026-8031).

Mouse knockout models lacking the PTP1B gene also point toward the negative regulation of insulin signaling by PTP1B. Mice harboring a disrupted PTP1B gene showed increased insulin sensitivity and increased phosphorylation of the insulin receptor. When placed on a high-fat diet, PTP1B−/− mice were resistant to weight gain and remained insulin sensitive (Elchebly et al., Science, 1999, 283, 1544-1548). These studies clearly establish PTP1B as a therapeutic target in the treatment of diabetes and obesity.

Diabetes and obesity (sometimes now collectively referred to as “diabesity”) are interrelated. Most human obesity is associated with insulin resistance and leptin resistance. In fact obesity may have an even greater impact on insulin action than does diabetes itself (Sindelka et al., Physiol Res., 2002, 51, 85-91). Syndrome X or metabolic syndrome is a new term for a cluster of conditions, that, when occurring together, may indicate a predisposition to diabetes and cardiovascular disease. These symptoms, including high blood pressure, high triglycerides, decreased HDL and obesity, tend to appear together in some individuals. Because of its role in both diabetes and obesity, PTP1B is believed to be a therapeutic target for a range of metabolic conditions, including diabetes, obesity and metabolic syndrome. By improving blood glucose control, inhibitors of PTP1B may also be useful in slowing, preventing, delaying or ameliorating the sequelae of diabetes, which include retinopathy, neuropathy, cardiovascular complications and nephropathy.

PTP1B, which is differentially regulated during the cell cycle (Schievella et al., Cell. Growth Differ., 1993, 4, 239-246), is expressed in insulin sensitive tissues as two different isoforms that arise from alternate splicing of the pre-mRNA (Shifrin and Neel, J. Biol. Chem., 1993, 268, 25376-25384). The ratio of the alternatively spliced products is affected by growth factors, such as insulin, and differs in various tissues examined (Sell and Reese, Mol. Genet. Metab., 1999, 66, 189-192). In these studies the levels of the variants correlated with the plasma insulin concentration and percentage body fat. These variants may therefore be used as a biomarker for patients with chronic hyperinsulinemia or type 2 diabetes.

PTP1B null mice are normal in size compared to their wild-type littermates and do not display increased incidence of tumor formation in old age compared to wild-type controls (Dube, N. PNAS, 2004 101:1834-1839). Signaling through several other growth factor receptors including epidermal growth factor receptor and insulin-like growth factor receptor, which is structurally homologous to the insulin receptor, was unchanged between PTP1B knockout and wild type mice.

Currently, therapeutic agents designed to inhibit the synthesis or action of PTP 1B include small molecules (Ham et al., Bioorg. Med. Chem. Lett., 1999, 9, 185-186; Skorey et al., J. Biol. Chem., 1997, 272, 22472-22480; Taing et al., Biochemistry, 1999, 38, 3793-3803; Taylor et al., Bioorg. Med. Chem., 1998, 6, 1457-1468; Wang et al., Bioorg. Med. Chem. Lett., 1998, 8, 345-350; Wang et al., Biochem. Pharmacol., 1997, 54, 703-711; Yao et al., Bioorg. Med. Chem., 1998, 6, 1799-1810) and peptides (Chen et al., Biochemistry, 1999, 38, 384-389; Desmarais et al., Arch. Biochem. Biophys., 1998, 354, 225-231; Roller et al., Bioorg. Med. Chem. Lett., 1998, 8, 2149-2150). In addition, International Patent Application Publication WO 97/32595 (Olefsky, 1997) refers to phosphopeptides and antibodies that inhibit the association of PTP1B with the activated insulin receptor for the treatment of disorders associated with insulin resistance, and refers to antisense nucleotides against PTP1B generally.

International Patent Application Publication WO 03/099227 (Lewis et al.) refers to small interfering RNAs (siRNAs) capable of interfering with expression of a PTP1B polypeptide, as well as pharmaceutical compositions and methods.

International Patent Application Publication WO 03/070881 (McSwiggen et al.) refers to short interfering nucleic acid (siNA) molecules that down-regulate expression of one or more PTP1B genes by RNA interference (RNAi), using short interfering nucleic acid (siNA), short interfering RNA (siRNA), double-stranded RNA (dsRNA), micro-RNA (miRNA), and short hairpin RNA (shRNA) molecules.

There remains a long felt need for additional agents and compositions capable of effectively inhibiting PTP1B function, in combination with other compounds, for the treatment of diabetes, obesity and related disorders.

### SUMMARY OF THE INVENTION

Provided herein is a method of reducing HbA_{1c} levels in a subject. In preferred embodiments, said subject is a human. In one embodiment, the method comprises administering to said subject an oligonucleotide having the nucleobase sequence “GCTCCTTCCACTGATCCTGC” (SEQ ID NO: 17) and which is targeted to PTP1B. In preferred embodiments, said oligonucleotide is administered in a dosing regimen comprised of a plurality of doses. In one embodiment, the subject has Type 2 diabetes, or, prior to the step of administering, said subject exhibits fasting blood glucose levels of at least 130 mg/dL, HbA_{1c} levels of at least 6%, or body mass index greater than 25 kg/m². In one embodiment, the subject has Type 2 diabetes, or, prior to the step of administering, said subject exhibits fasting blood glucose levels of at least 130 mg/dL, HbA_{1c} levels of at least 6.8%, or body mass index greater than 25 kg/m². In one embodiment, said subject exhibits HbA_{1c} levels of at least about 6%, at least about 7%, at least about 8%, at least about 9%, at least about 10% or at least about 11%. In preferred embodiments, said subject does not achieve normal glucose levels on a therapeutic regimen of insulin, sulfonylurea, or metformin. In preferred embodiments, HbA_{1c} levels are reduced to about 7% or below about 7%. In some embodiments, doses are administered approximately daily, weekly, biweekly, or monthly. In one embodiment, each dose of said plurality of doses comprises from about 0.5 to about 7.5 mg/kg of the oligonucleotide. In a preferred embodiment, each dose of said plurality of doses comprises from about 100 to about 200 mg of the oligonucleotide. In other preferred embodiments, each does of said plurality of doses comprises about 400 mg of the oligonucleotide. In preferred embodiments, the oligonucleotide is characterized by a ten-deoxynucleotide gap region flanked on its 3′ and 5′ ends with five 2′-O-(2-methoxyethyl) nucleotides, and wherein all the cytosines nucleotides are optionally 5-methylcytosines or at least one internucleoside linkage is a phosphorothioate linkage. All cytosines may be 5-methylcytosines, and each internucleoside linkage may be a phosphorothioate, or both. The term “ISIS 113715,” as used herein, refers to an oligonucleotide of SEQ ID NO: 17 having a ten-deoxynucleotide gap region flanked on its 3′ and 5′ ends with five 2′-O-(2-methoxyethyl) nucleotides, and wherein all the cytosines nucleotides are 5-methylcytosines and each internucleoside linkage is a phosphorothioate linkage.

Further provided herein is a method of reducing fasting glucose levels in a subject. In preferred embodiments, said subject is a human. In one embodiment, the method comprises administering to said subject an oligonucleotide having the nucleobase sequence “GCTCCTTCCACTGATCCTGC” (SEQ ID NO: 17) and which is targeted to PTP1B. Fasting glucose may be fasting blood glucose, fasting serum glucose, or fasting plasma glucose. In preferred embodiments, said oligonucleotide is administered in a dosing regimen comprised of a plurality of doses. In some embodiments, fasting plasma glucose levels are reduced by at least about 25 mg/dL or by at least about 10 mg/dL. In one embodiment, the subject has Type 2 diabetes, or, prior to the step of administering, said subject exhibits fasting blood glucose levels of at least 130 mg/dL, HbA_{1c} levels of at least 6%, or body mass index greater than 25 kg/m². In one embodiment, the subject has Type 2 diabetes, or, prior to the step of administering, said subject exhibits fasting blood glucose levels of at least 130 mg/dL, HbA_{1c} levels of at least 6.8%, or body mass index greater than 25 kg/m². In preferred embodiments, said subject does not achieve normal glucose levels on a therapeutic regimen of insulin, sulfonylurea, or metformin. In preferred embodiments, HbA_{1c} levels are reduced to about 7% or below about 7%. In some embodiments, doses are administered approximately daily, weekly, biweekly, or monthly. In one embodiment, each dose of said plurality of doses comprises from about 0.5 to about 7.5 mg/kg of the oligonucleotide. In a preferred embodiment, each dose of said plurality of doses comprises from about 100 to about 200 mg of the oligonucleotide. In preferred embodiments, the oligonucleotide is characterized by a ten-deoxynucleotide gap region flanked on its 3′ and 5′ ends with five 2′-O-(2-methoxyethyl) nucleotides, and wherein the cytosine nucleotides are optionally 5-methylcytosines or at least one internucleoside linkage is a phosphorothioate linkage. All cytosines may be 5-methylcytosines, and each internucleoside linkage may be a phosphorothioate, or both. In an additional embodiment, the oligonucleotide is ISIS 113715.

Also provided are methods of reducing adiposity, apolipoprotein B levels, LDL levels, cholesterol levels, triglyceride levels, VLDL levels, or LDL: HDL ratios or cholesterol:HDL ratios in a subject. Also provided are methods of increasing adiponectin levels, metabolic rate, HDL levels or HDL:LDL ratios or HDL:cholesterol ratios in a subject. In preferred embodiments, said subject is a human. Also provided is a method of treating obesity wherein metabolic rate is increased. In preferred embodiments, the method comprises administering to said subject a plurality of doses of an oligonucleotide having the nucleobase sequence “GCTCCTTCCACTGATCCTGC” (SEQ ID NO: 17) and which is targeted to PTP1B. In preferred embodiments, said oligonucleotide is administered in a dosing regimen comprised of a plurality of doses. In some embodiments, fasting plasma glucose levels are reduced by at least about 25 mg/dL or by at least about 10 mg/dL. In some embodiments, doses are administered approximately weekly, biweekly, or daily. In one embodiment, each dose of said plurality of doses comprises from about 0.5 to about 7.5 mg/kg of the oligonucleotide. In a preferred embodiment, each dose of said plurality of doses comprises from about 100 to about 200 mg of the oligonucleotide. In preferred embodiments, the oligonucleotide is characterized by a ten-deoxynucleotide gap region flanked on its 3′ and 5′ ends with five 2′-O-(2-methoxyethyl) nucleotides, and wherein the cytosine nucleotides are optionally 5-methylcytosines or at least one internucleoside linkage is a phosphorothioate linkage. All cytosines may be 5-methylcytosines, and each internucleoside linkage may be a phosphorothioate, or both.

Also contemplated are methods of reducing fasting glucose or HbA_{1c} levels or altering lipid levels, or a combination thereof in a subject comprising administering to said animal an oligonucleotide comprising the nucleobase sequence “GCTCCTTCCACTGATCCTGC” (SEQ ID NO: 17) and which is targeted to PTP1B wherein said oligonucleotide is administered during a loading period and a maintenance period. In some embodiments, the oligonucleotide having the nucleobase sequence “GCTCCTTCCACTGATCCTGC” (SEQ ID NO: 17) and which is targeted to PTP1B and is characterized by a ten-deoxynucleotide gap region flanked on its 3′ and 5′ ends with five 2′-O-(2-methoxyethyl) nucleotides is administered by injection or orally. In some embodiments, the oligonucleotide is administered by intravenous or subcutaneous injection. In a preferred embodiment, the subject is a human. In some embodiments, the loading period results in at least 70-80% of steady-state levels of oligonucleotide in organs. In some embodiments, the loading period comprises administering the oligonucleotide to the subject once per day for up to 10 days, once per week for about 3 weeks, or twice per week for about 3 weeks. In some embodiments, the oligonucleotide is delivered intravenously during the loading period. In other embodiments, the oligonucleotide is delivered subcutaneously during the loading period. In some embodiments, the oligonucleotide is delivered subcutaneously during the maintenance period. In some embodiments, the oligonucleotide is delivered subcutaneously in at least one injection site per administration. In some embodiments, the injection site is in the abdomen. In some embodiments, the oligonucleotide is delivered subcutaneously in more than one injection site per administration. In some embodiments, the oligonucleotide is delivered subcutaneously in more than one injection site per administration, and wherein no two consecutive injections are in injection sites in the same quadrant of the abdomen.

In one embodiment, the maintenance period comprises administering the oligonucleotide at least about once a week. In one embodiment, the dosing regimen for the loading period results in at least about 70 to 80% of steady-state organ levels during the first week of treatment.

In some embodiments of the present invention, the subject exhibits hyperglycemia prior to the start of treatment or exhibits fasting blood glucose levels above about 130 mg/dL, baseline HbA_{1c} levels of at least about 7%, or body mass index of greater than 25 kg/m².

The methods provided herein may further comprise administration of another glucose-lowering therapeutic. In some embodiments, said glucose-lowering therapeutic is a PPAR agonist (gamma, dual, or pan), a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor. In some embodiments, the additional glucose-lowering therapeutic is metformin, sulfonylurea, or rosiglitazone.

Provided herein are methods of treating hyperglycemia, Type 2 diabetes, prediabetes, metabolic syndrome, or obesity in a subject comprising administering to said subject a combination therapy comprising at least one glucose-lowering therapeutic and an oligonucleotide having the nucleobase sequence “GCTCCTTCCACTGATCCTGC” (SEQ ID NO: 17) and which is targeted to PTP1B wherein said oligonucleotide is administered during a loading period and a maintenance period. Also contemplated are methods of decreasing blood glucose with such a combination therapy. The glucose-lowering therapeutic may be a PPAR agonist (gamma, dual or pan), a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor. In some embodiments, the glucose-lowering therapeutic is metformin, sulfonylurea, or rosiglitazone. In some embodiments, the glucose-lowering therapeutic is a GLP-1 analog.

In some embodiments, the GLP-1 analog is exendin-4 or liraglutide. In other embodiments, the glucose-lowering therapeutic is a sulfonylurea. In some embodiments, the sulfonylurea is acetohexamide, chlorpropamide, tolbutamide, tolazamide, glimepiride, a glipizide, a glyburide, or a gliclazide. In some embodiments, the glucose lowering drug is a biguanide. In some embodiments, the biguanide is metformin, and in some embodiments, blood glucose levels are decreased without increased lactic acidosis as compared to the lactic acidosis observed after treatment with metformin alone. In some embodiments, the glucose lowering drug is a meglitinide. In some embodiments, the meglitinide is nateglinide or repaglinide. In some embodiments, the glucose-lowering drug is a thiazolidinedione. In some embodiments, the thiazolidinedione is pioglitazone, rosiglitazone, or troglitazone. In some embodiments, blood glucose levels are decreased without greater weight gain than observed with rosiglitazone treatment alone.

In some embodiments, the glucose-lowering drug is an alpha-glucosidase inhibitor. In some embodiments, the alpha-glucosidase inhibitor is acarbose or miglitol. In some embodiments, the glucose-lowering therapeutic is insulin or an insulin analog.

Also provided are methods of treating hyperglycemia, prediabetes, Type 2 diabetes, metabolic syndrome, or obesity in a subject comprising administering to said subject a combination therapy comprising at least one lipid-lowering therapeutic and an oligonucleotide having the nucleobase sequence “GCTCCTTCCACTGATCCTGC” (SEQ ID NO: 17) and which is targeted to PTP1B wherein said oligonucleotide is administered during a loading period and a maintenance period. Further provided are methods of treating hyperglycemia, prediabetes, Type 2 diabetes, metabolic syndrome, or obesity in a subject comprising administering to said subject a combination therapy comprising at least one anti-obesity therapeutic and an oligonucleotide having the nucleobase sequence “GCTCCTTCCACTGATCCTGC” (SEQ ID NO: 17) and which is targeted to PTP1B wherein said oligonucleotide is administered during a loading period and a maintenance period. Also provided are methods of treating prediabetes hyperglycemia, Type 2 diabetes, metabolic syndrome, or obesity in a subject comprising administering to said an oligonucleotide having the nucleobase sequence “GCTCCTTCCACTGATCCTGC” (SEQ ID NO: 17) and which is targeted to PTP1B wherein said oligonucleotide is administered via injection and further comprising administering a topical steroid at the injection site.

The present invention also provides a vial containing ISIS 113715 as a 10 mg/mL, 200 mg/mL or 250 mg/mL sterile solution. In one embodiment, the vial contains a 10 mg/mL solution of ISIS 113715 which contains phosphate buffer, sodium chloride, and water and is isotonic. In another embodiment, the vial contains a 200 mg/mL solution of ISIS 113715 which contains water and is hypertonic. In another embodiment, the vial contains a 250 mg/mL solution of ISIS 113715 which contains water and is hypertonic. In some embodiments, the vial also contains a preservative. In some embodiments, the preservative is metacresol.

The present invention also provides a vial containing ISIS 113715 as sterile lyophilized powder. In one embodiment, the vial contains 150 mg of ISIS 113715. In another embodiment, the vial is supplied with a sterile preserved diluent. In another embodiment, the sterile preserved diluent comprises 0.1-1.0% metacresol. In a preferred embodiment, the sterile preserved diluent comprises 0.3% metacresol.

Further provided herein is a pharmaceutical composition comprising one or more doses of an oligonucleotide having the nucleobase sequence “GCTCCTTCCACTGATCCTGC” (SEQ ID NO: 17) and which is targeted to PTP1B, wherein each of said one or more doses ranges from about 50 mg to about 900 mg, and wherein subcutaneous administration to a subject of said oligonucleotide at about 0.5 mg/kg of body weight to about 7.5 mg/kg of body weight subsequent to the administration of one or more loading doses is sufficient to achieve a plasma absolute bioavailability of at least about 32%. In some embodiments, the administration of said pharmaceutical composition occurs at least once daily, at least once weekly, or at least once monthly.

Also provided herein are uses of an oligonucleotide having the nucleobase sequence “GCTCCTTCCACTGATCCTGC” (SEQ ID NO: 17) and which is targeted to PTP1B for the preparation of a medicament for reducing blood glucose levels, wherein said medicament is administered during a loading period and a maintenance period. In some embodiments, the medicament is administered subcutaneously or intravenously. In other embodiments, the administration of said medicament occurs at least once daily, at least once weekly, or at least once monthly. In some embodiments, oligonucleotide present in the medicament is administered in a dose from about 50 mg to about 900 mg. Said medicament may be administered to a subject that exhibits Type 2 diabetes, metabolic syndrome, or obesity.

In one aspect, the present invention is directed to compositions and methods for decreasing blood glucose levels in an animal or for preventing or delaying the onset of a rise in blood glucose levels in an animal, comprising administering to said animal an antisense inhibitor of PTP 1B expression in combination with at least one glucose-lowering drug.

In another aspect, the present invention is also directed to compositions and methods for improving insulin sensitivity in an animal or for preventing or delaying the onset of insulin resistance in an animal, comprising administering to said animal an antisense inhibitor of PTP1B expression in combination with at least one glucose-lowering drug.

In a further aspect, the present invention is further directed to compositions and methods for treating a metabolic condition in an animal or for preventing or delaying the onset of a metabolic condition in an animal, comprising administering to said animal an antisense inhibitor of PTP1B expression in combination with at least one glucose-lowering drug. The metabolic condition may be, e.g., diabetes or obesity.

Other embodiments of the present invention include methods of reducing cholesterol, LDL and VLDL levels in an animal comprising administering to said animal an antisense inhibitor of PTP1B expression. Another embodiment of the present invention is a method of increasing HDL levels in an animal comprising administering to said animal an antisense inhibitor of PTP1B. Another embodiment of the present invention is a method of reducing LDL:HDL ratio or total cholesterol:HDL ratio in an animal comprising administering to said animal an antisense inhibitor of PTP 1B. Another embodiment of the present invention is a method of increasing HDL:LDL ratio or HDL:total cholesterol ratio in an animal comprising administering to said animal an antisense inhibitor of PTP1B. Another embodiment of the present invention is a method of improving lipid profile in an animal comprising increasing HDL, lowering LDL, lowering VLDL, lowering triglycerides, lowering apolipoprotein B levels, or lowering total cholesterol levels, or a combination thereof.

In preferred embodiments, the antisense inhibitor of PTP1B has the nucleobase sequence of SEQ ID NO: 17. In other preferred embodiments, the antisense inhibitor of PTP1B is ISIS 113715.

Other aspects and advantages are disclosed in the following detailed description of the invention.

### BRIEF DESCRIPTION OF DRAWINGS

FIG. 1. Treatment of patients with Type 2 diabetes with ISIS 113715 results in a decrease in HbA_{1c} levels. Shown are analysis of covariance results for screening adjusted treatment difference from placebo. The difference in HbA_{1c} levels and the 95% confidence interval is shown for data pooled from the 100 mg and 200 mg dose cohorts from CS-7 which is described herein.

FIG. 2. Treatment of patients with Type 2 diabetes with ISIS 113715 results in a decrease in HbA_{1c} levels after 6 weeks of treatment. Shown are analysis of covariance results for screening adjusted treatment difference from placebo. The difference in HbA_{1c} levels and the 95% confidence interval is shown for both the 100 mg and 200 mg dose cohorts from CS-7 which is described herein.

FIG. 3. Treatment of patients with Type 2 diabetes with ISIS 113715 results in a decrease in HbA_{1c} levels which outlives any placebo effect. The median percent change in Cohort C (400 mg) HbA_{1c} levels from baseline measurements is greater than that observed for placebo-treated patients, and decreases in HbA_{1c} levels continue in the treatment group while the initial decline plateaus for the placebo group. Data shown are from CS-7.

FIG. 4. Treatment with ISIS 113715 results in parallel decreases in fasting serum glucose and HbA_{1c} levels. Shown are FSG and HbA_{1c} levels for a patient from Cohort A (100 mg) and for a patient from Cohort B (200 mg) from CS-7.

FIG. 5. Treatment with ISIS 113715 results in parallel decreases in fasting serum glucose and HbA_{1c} levels. Shown are FSG and HbA_{1c} levels for a patient from Cohort C (400 mg) of CS-7.

FIG. 6. Fasting plasma glucose (FPG) is decreased in patients with Type 2 diabetes treated with ISIS 113715. Shown are analysis of covariance results for screening adjusted treatment difference from placebo. The difference in fasting plasma glucose levels and the 95% confidence interval is shown for data pooled from the 100 mg and 200 mg dose cohorts from CS-7 as compared to placebo.

FIG. 7. Fasting plasma glucose (FPG) is decreased in patients with Type 2 diabetes treated with ISIS 113715. Shown are analysis of covariance results for screening adjusted treatment difference from placebo. The difference in fasting plasma glucose levels and the 95% confidence interval is shown for data from the 100 mg and 200 mg dose cohorts from CS-7 as compared to placebo.

FIG. 8. Treatment with ISIS 113715 causes alterations in lipid profile in patients with Type 2 diabetes. The effects of ISIS 113715 on lipids is shown in the analysis of covariance results. Baseline adjusted lipid differences from placebo are shown for the 100 mg, 200 mg, and 400 mg cohorts from CS-7.

FIG. 9. ISIS 113715 reduces apoB-100, serum cholesterol, and serum LDL in obese monkeys.

FIG. 10. ISIS 113715 increases metabolic rate in mice fed a high-fat diet. Mice fed a high-fat diet (60% fat) were treated with 25 mg/kg of ISIS 113715 twice per week for five weeks. As shown, VO2 consumption (mL/g/h) was increased in animals treated with ISIS 113715, consistent with an increased metabolic rate. Metabolic rate was measured using indirect calorimetry methods known in the art (for example, using the Oxymax system, Columbus Instruments, Columbus, Ohio).

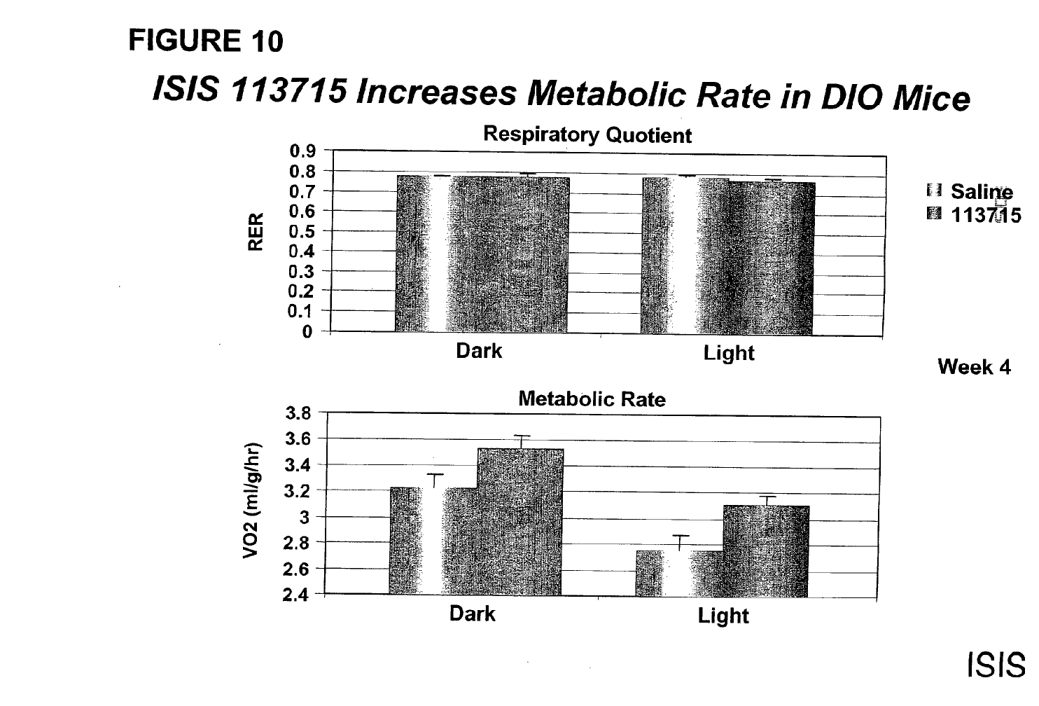

### DETAILED DESCRIPTION OF THE INVENTION

Certain embodiments of the present invention are described in the following numbered paragraphs:
1. A method of reducing HbA_{1c} levels in a subject, comprising administering to said subject an oligonucleotide having the nucleobase sequence “GCTCCTTCCACTGATCCTGC” (SEQ ID NO: 17) and which is targeted to PTP1B.
2. The method of paragraph 1 wherein said oligonucleotide is administered in a dosing regimen comprised of a plurality of doses.
3. The method of paragraph 1 wherein said subject has Type 2 diabetes.
4. The method of paragraph 1 wherein, prior to the step of administering, said subject exhibits fasting blood glucose levels of at least 130 mg/dL, HbA_{1c} levels of at least 6%, or body mass index greater than 25 kg/m².
5. The method of paragraph 4 wherein said subject does not achieve normal glucose levels on a therapeutic regimen of insulin, sulfonylurea, or metformin.
6. The method of paragraph 1 wherein HbA, levels are reduced to about 7%.
7. The method of paragraph I wherein HbA_{1c} levels are reduced to about 7% or below about 7%.
8. The method of paragraph 2 wherein said doses are administered approximately weekly.
9. The method of paragraph 2 wherein said doses are administered approximately biweekly.
10. The method of paragraph 2 wherein said doses are administered daily.
11. The method of paragraph 1 wherein each dose of said plurality of doses comprises from about 0.5 to about 7.5 mg/kg of the oligonucleotide.
12. The method of paragraph 1 wherein each dose of said plurality of doses comprises from about 100 to about 200 mg of the oligonucleotide.
13. The method of any one of paragraphs 1-12 wherein said oligonucleotide is characterized by a ten-deoxynucleotide gap region flanked on its 3′ and 5′ ends with five 2′-O-(2-methoxyethyl) nucleotides, and wherein all cytosines are 5-methylcytosines or at least one internucleoside linkage is a phosphorothioate linkage.
14. The method of paragraph 13 wherein all cytosines are 5-methylcytosines.
15. The method of paragraph 13 wherein each internucleoside linkage is a phosphorothioate.
16. A method of reducing fasting glucose levels in a subject, comprising administering to said subject an oligonucleotide having the nucleobase sequence “GCTCCTTCCACTGATCCTGC” (SEQ ID NO: 17) and which is targeted to PTP1B.
17. The method of paragraph 16 wherein fasting glucose is fasting blood glucose, fasting serum glucose, or fasting plasma glucose.
18. The method of paragraph 16 wherein said oligonucleotide is administered in a dosing regimen comprised of a plurality of doses.
19. The method of paragraph 16 wherein fasting plasma glucose levels are reduced by at least about 25 mg/dL.
20. The method of paragraph 16 wherein fasting plasma glucose levels are reduced by at least about 10 mg/dL.
21. The method of paragraph 16 wherein at least one dose of said plurality of doses is administered about once a week.
22. The method of paragraph 16 wherein at least one dose of said plurality of doses is administered about once every other week.
23. The method of paragraph 16 wherein at least one dose of said plurality of doses is administered about once a day.
24. The method of paragraph 16 wherein each dose of said plurality of doses comprises from about 0.5 to about 7.5 mg/kg.
25. The method of paragraph 16 wherein each dose of said plurality of doses comprises from about 100 to about 200 mg of oligonucleotide per week.
26. The method of any one of paragraphs 16-25 wherein said oligonucleotide is characterized by a ten-deoxynucleotide gap region flanked on its 3′ and 5′ ends with five 2′-O-(2-methoxyethyl) nucleotides, all cytosines are 5-methylcytosines, and each internucleoside linkage is a phosphorothioate linkage.
27. A method of increasing metabolic rate in a subject comprising administering to said subject a plurality of doses of an oligonucleotide having the nucleobase sequence “GCTCCTTCCACTGATCCTGC” (SEQ ID NO: 17) and which is targeted to PTP1B.
28. The method of paragraph 27 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
29. A method of reducing LDL levels in a subject comprising administering to said subject a plurality of doses of an oligonucleotide having the nucleobase sequence “GCTCCTTCCACTGATCCTGC” (SEQ ID NO: 17) and which is targeted to PTP1B.
30. The method of paragraph 29 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
31. A method of reducing cholesterol levels in a subject comprising administering to said subject a plurality of doses of an oligonucleotide having the nucleobase sequence “GCTCCTTCCACTGATCCTGC” (SEQ ID NO: 17) and which is targeted to PTP1B.
32. The method of paragraph 31 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
33. A method of increasing HDL levels in a subject comprising administering to said subject a plurality of doses of an oligonucleotide having the nucleobase sequence “GCTCCTTCCACTGATCCTGC” (SEQ ID NO: 17) and which is targeted to PTP1B.
34. The method of paragraph 33 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
35. A method of increasing the HDL:LDL ratio in a subject comprising administering to said subject a plurality of doses of an oligonucleotide having the nucleobase sequence “GCTCCTTCCACTGATCCTGC” (SEQ ID NO: 17) and which is targeted to PTP1B.
36. The method of paragraph 35 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
37. A method of decreasing circulating triglycerides in a subject comprising administering to said subject a plurality of doses of an oligonucleotide having the nucleobase sequence “GCTCCTTCCACTGATCCTGC” (SEQ ID NO: 17) and which is targeted to PTP1B.
38. The method of paragraph 37 wherein said oligonucleotide having the nucleobase sequence “GCTCCTTCCACTGATCCTGC” (SEQ ID NO: 17) and which is targeted to PTP1B and is characterized by a ten-deoxynucleotide gap region flanked on its 3′ and 5′ ends with five 2′-O-(2-methoxyethyl) nucleotides.
39. A method of decreasing adiposity in a subject comprising administering to said subject a plurality of doses of an oligonucleotide having the nucleobase sequence “GCTCCTTCCACTGATCCTGC” (SEQ ID NO: 17) and which is targeted to PTP1B.
40. The method of paragraph 39 wherein said oligonucleotide having the nucleobase sequence “GCTCCTTCCACTGATCCTGC” (SEQ ID NO: 17) and which is targeted to PTP1B is characterized by a ten-deoxynucleotide gap region flanked on its 3′ and 5′ ends with five 2′-O-(2-methoxyethyl) nucleotides.
41. A method of reducing fasting glucose or HbA_{1c} levels in an animal comprising administering to said animal an oligonucleotide comprising the nucleobase sequence “GCTCCTTCCACTGATCCTGC” (SEQ ID NO: 17) and which is targeted to PTP1B wherein said oligonucleotide is administered during a loading period and a maintenance period.
42. The method of paragraph 41 wherein the loading period results in at least 70-80% steady-state levels of oligonucleotide in organs.
43. The method of paragraph 41 wherein the loading period comprises administering the oligonucleotide to the subject once per day for up to 10 days.
44. The method of paragraph 41 wherein the loading period comprises administering the oligonucleotide to the subject about once per week for about 3 weeks.
45. The method of paragraph 41 wherein the loading period comprises administering the oligonucleotide to the subject about twice per week for about 3 weeks.
46. The method of paragraph 41 wherein the oligonucleotide is delivered intravenously during the loading period.
47. The method of paragraph 41 wherein the oligonucleotide is delivered subcutaneously during the loading period.
48. The method of paragraph 41 wherein the oligonucleotide is delivered subcutaneously during the maintenance period.
49. The method of paragraph 41 wherein the oligonucleotide is delivered subcutaneously in at least one injection site per administration.
50. The method of paragraph 41 wherein the oligonucleotide is delivered subcutaneously in at least one injection site per administration, and wherein the injection site is in the abdomen.
51. The method of paragraph 41 wherein the oligonucleotide is delivered subcutaneously in more than one injection site per administration.
52. The method of paragraph 41 wherein the oligonucleotide is delivered subcutaneously in more than one injection site per administration, and wherein no two consecutive injections are in injection sites in the same quadrant of the abdomen.
53. The method of paragraph 41 wherein the maintenance period comprises administering the oligonucleotide at least about once a week.
54. The method of paragraph 41 wherein the dosing regimen for the loading period results in at least about 70 to 80% of steady-state organ levels during the first week of treatment.
55. The method of paragraph 41 wherein said subject exhibits hyperglycemia prior to the start of treatment.
56. The method of paragraph 41 wherein said subject exhibits fasting blood glucose levels above about 130 mg/dL, baseline HbA_{1c} levels of at least about 7%, or body mass index of greater than 25 kg/m².
57. The method of paragraph 41 further comprising administration of another glucose-lowering drug.
58. The method of paragraph 57 wherein said glucose-lowering drug is a PPAR agonist, a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor.
59. The method of paragraph 57 wherein said glucose-lowering drug is metformin, sulfonylurea, or rosiglitazone.
60. A method of treating Type 2 diabetes, metabolic syndrome, or obesity in a subject comprising administering to said subject a combination therapy comprising at least one glucose-lowering drug and an oligonucleotide having the nucleobase sequence “GCTCCTTCCACTGATCCTGC” (SEQ ID NO: 17) and which is targeted to PTP1B wherein said oligonucleotide is administered during a loading period and a maintenance period.
61. The method of paragraph 60 wherein said glucose-lowering drug is a PPAR agonist, a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor.
62. The method of paragraph 60 wherein said glucose-lowering drug is metformin, sulfonylurea, or rosiglitazone.
63. A method of decreasing blood glucose levels in a subject comprising administering to said subject an glucose-lowering drug in combination with an oligonucleotide having the nucleobase sequence “GCTCCTTCCACTGATCCTGC” (SEQ ID NO: 17) and which is targeted to PTP1B and is characterized by a ten-deoxynucleotide gap region flanked on its 3′ and 5′ ends with five 2′-O-(2-methoxyethyl) nucleotides.
64. The method of paragraph 63 wherein said glucose-lowering drug is a PPAR agonist, a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor.
65. The method of paragraph 63 wherein said glucose-lowering drug is a GLP-1 analog.
66. The method of paragraph 65 wherein the GLP-1 analog is exendin-4 or liraglutide.
67. The method of paragraph 63 wherein said glucose-lowering drug is a sulfonylurea.
68. The method of paragraph 67 wherein the sulfonylurea is acetohexamide, chlorpropamide, tolbutamide, tolazamide, glimepiride, a glipizide, a glyburide, or a gliclazide.
69. The method of paragraph 63 wherein the glucose lowering drug is a biguanide.
70. The method of paragraph 69 wherein the biguanide is metformin.
71. The method of paragraph 70 wherein blood glucose levels are decreased without increased lactic acidosis as compared to the lactic acidosis observed after treatment with metformin alone.
72. The method of paragraph 63 wherein the glucose lowering drug is a meglitinide.
73. The method of paragraph 72 wherein the meglitinide is nateglinide or repaglinide.
74. The method of paragraph 63 wherein the glucose-lowering drug is a thiazolidinedione.
75. The method of paragraph 74 wherein the thiazolidinedione is pioglitazone, rosiglitazone, or troglitazone.
76. The method of paragraph 75 wherein blood glucose levels are deceased without greater weight gain than observed with rosiglitazone alone.
77. The method of paragraph 63 wherein the glucose-lowering drug is an alpha-glucosidase inhibitor.
78. The method of paragraph 77 wherein the alpha-glucosidase inhibitor is acarbose or miglitol.
79. The method of paragraph 63 wherein the glucose-lowering drug is insulin or an insulin analog.
80. The method of paragraph 63 wherein the oligonucleotide having the nucleobase sequence “GCTCCTTCCACTGATCCTGC” (SEQ ID NO: 17) and which is targeted to PTP1B and is characterized by a ten-deoxynucleotide gap region flanked on its 3′ and 5′ ends with five 2′-O-(2-methoxyethyl) nucleotides is administered by injection or orally.
81. The method of paragraph 63 wherein the oligonucleotide having the nucleobase sequence “GCTCCTTCCACTGATCCTGC” (SEQ ID NO: 17) and which is targeted to PTP1B and is characterized by a ten-deoxynucleotide gap region flanked on its 3′ and 5′ ends with five 2′-O-(2-methoxyethyl) nucleotides is administered by intravenous or subcutaneous injection.
82. A method of treating hyperglycemia, Type 2 diabetes, metabolic syndrome, or obesity in a subject comprising administering to said subject a combination therapy comprising at least one lipid-lowering drug and an oligonucleotide having the nucleobase sequence “GCTCCTTCCACTGATCCTGC” (SEQ ID NO: 17) and which is targeted to PTP 1B wherein said oligonucleotide is administered during a loading period and a maintenance period.
83. A method of treating hyperglycemia, Type 2 diabetes, metabolic syndrome, or obesity in a subject comprising administering to said subject a combination therapy comprising at least one anti-obesity drug and an oligonucleotide having the nucleobase sequence “GCTCCTTCCACTGATCCTGC” (SEQ ID NO: 17) and which is targeted to PTP1B wherein said oligonucleotide is administered during a loading period and a maintenance period.
84. A method of treating hyperglycemia, Type 2 diabetes, metabolic syndrome, or obesity in a subject comprising administering to said an oligonucleotide having the nucleobase sequence “GCTCCTTCCACTGATCCTGC” (SEQ ID NO: 17) and which is targeted to PTP1B wherein said oligonucleotide is administered via injection and further comprising administering a topical steroid at the injection site.
85. A vial containing ISIS 113715 as a 10 mg/mL, 200 mg/mL or 250 mg/mL sterile solution.
86. The vial of paragraph 85 containing a 10 mg/mL solution of ISIS 113715 which contains phosphate buffer, sodium chloride, and water and is isotonic.
87. The vial of paragraph 85 containing a 200 mg/mL solution of ISIS 113715 which contains water and is hypertonic.
88. The vial of paragraph 85 containing a 250 mg/mL solution of ISIS 113715 which contains water and is hypertonic.
89. The vial of paragraph 85 also containing a preservative.
90. The vial of paragraph 89 wherein said preservative is metacresol.
91. A vial containing ISIS 113715 as sterile lyophilized powder.
92. The vial of paragraph 91 wherein said vial contains 150 mg of ISIS 113715.
93. The vial of paragraph 91 supplied with a sterile preserved diluent.
94. The vial of paragraph 93 wherein the sterile preserved diluent comprises 0.3% metacresol.
95. A pharmaceutical composition comprising one or more doses of an oligonucleotide having the nucleobase sequence “GCTCCTTCCACTGATCCTGC” (SEQ ID NO: 17) and which is targeted to PTP1B, wherein each of said one or more doses ranges from about 50 mg to about 900 mg, and wherein subcutaneous administration to a subject of said oligonucleotide at about 0.5 mg/kg of body weight to about 7.5 mg/kg of body weight subsequent to the administration of one or more loading doses is sufficient to achieve an absolute plasma bioavailability of at least about 32%.
96. Use of an oligonucleotide having the nucleobase sequence “GCTCCTTCCACTGATCCTGC” (SEQ ID NO: 17) and which is targeted to PTP1B for the preparation of a medicament for reducing blood glucose levels, wherein said medicament is administered during a loading period and a maintenance period.
97. The use of paragraph 96 wherein the administration of said medicament occurs at least once daily.
98. The use of paragraph 96 wherein the administration of said medicament occurs at least once weekly.
99. The use of paragraph 96 wherein the administration of said medicament occurs at least once monthly.
100. The use of paragraph 96 wherein said medicament is administered subcutaneously or intravenously.
101. The use of paragraph 96 wherein the administration of said medicament occurs at least once daily.
102. The use of paragraph 96 wherein the administration of said medicament occurs at least once weekly.
103. The use of paragraph 96 wherein the administration of said medicament occurs at least once monthly.
104. The use of paragraph 96 wherein the oligonucleotide present in the medicament is administered in a dose from about 50 mg to about 900 mg.
105. The use of paragraph 96 wherein said medicament is administered to a subject that exhibits hyperglycemia, Type 2 diabetes, metabolic syndrome, or obesity.

For therapeutics, an animal, preferably a human, suspected of having a disease or disorder which can be treated by modulating the expression of PTP1B is treated by administering antisense compounds, particularly ISIS 113715, in accordance with this invention. For example, in one non-limiting embodiment, the methods comprise the step of administering to an animal a therapeutically effective amount of a PTP1B inhibitor. The PTP1B inhibitors of the present invention effectively inhibit the activity of the PTP1B protein or inhibit the expression of the PTP1B protein. In one embodiment, the activity or expression of PTP1B in an animal is inhibited by about 10%. Preferably, the activity or expression of PTP1B in an animal is inhibited by about 30%. More preferably, the activity or expression of PTP1B in an animal is inhibited by 50% or more. Thus, the oligomeric antisense compounds modulate expression of PTP1B mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

Preferably, the cells within said fluids, tissues or organs being analyzed contain a nucleic acid molecule encoding PTP1B protein and/or the PTP1B protein itself. Samples of organs or tissues may be obtained through routine clinical biopsy. Samples of bodily fluid such as blood or urine are routinely and easily tested. For example blood glucose levels can be determined by a physician or even by the patient using a commonly available test kit or glucometer (for example, the Ascensia ELITE™ kit, Ascensia (Bayer), Tarrytown N.Y., or Accucheck, Roche Diagnostics). Alternatively or in addition, glycated hemoglobin (HbA_{1c}) may be measured. HbA, is a stable minor hemoglobin variant formed in vivo via posttranslational modification by glucose, and it contains predominantly glycated NH₂-terminal β-chains. There is a strong correlation between levels of HbA_{1c} and the average blood glucose levels over the previous 3 months. Thus HbA_{1c} is often viewed as the “gold standard” for measuring sustained blood glucose control (Bunn, H. F. et al., 1978, Science. 200, 21-7). HbA_{1c} can be measured by ion-exchange HPLC or immunoassay; home blood collection and mailing kits for HbA_{1c} measurement are now widely available. Serum fructosamine is another measure of stable glucose control and can be measured by a colorimetric method (Cobas Integra, Roche Diagnostics).

Because ISIS 113715 has been shown to be useful in, for example, lowering blood glucose and improving insulin sensitivity, it is useful in treating metabolic conditions, particularly those associated with insulin resistance and/or elevated blood glucose; such as type 2 diabetes. Use of ISIS 113715 and methods of the invention is useful prophylactically, e.g., to prevent or delay the progression or development of diabetes or elevated blood glucose levels, for example.

Because ISIS 113715 is shown herein to increase insulin sensitivity in normal animals fed a high-fat diet, and to reduce weight gain of these animals, ISIS 113715 is useful in treating, preventing or delaying insulin resistance and weight gain.

ISIS 113715 can be utilized in pharmaceutical compositions by adding an effective amount of a compound to a suitable pharmaceutically acceptable diluent or carrier. Use of the compounds and methods of the invention may also be useful prophylactically to prevent such diseases or disorders, e.g., to prevent or delay undue weight gain, or diabetes.

### Metabolic Syndrome

“Metabolic syndrome” is defined as a clustering of lipid and non-lipid cardiovascular risk factors of metabolic origin. It has been closely linked to the generalized metabolic disorder known as insulin resistance. The National Cholesterol Education Program (NCEP) Adult Treatment Panel III (ATPIII) established criteria for diagnosis of metabolic syndrome when three or more of five risk determinants are present. The five risk determinants are abdominal obesity defined as waist circumference of greater than 102 cm for men or greater than 88 cm for women, triglyceride levels greater than or equal to 150 mg/dL, HDL cholesterol levels of less than 40 mg/dL for men and less than 50 mg/dL for women, blood pressure greater than or equal to 130/85 mm Hg and fasting glucose levels greater than or equal to 110 mg/dL. These determinants can be readily measured in clinical practice (JAMA, 2001, 285: 2486-2497).

The World Health Organization definition of metabolic syndrome is diabetes, impaired fasting glucose, impaired glucose tolerance, or insulin resistance (assessed by clamp studies) and at least two of the following criteria: waist-to-hip ratio greater than 0.90 in men or greater than 0.85 in women, serum triglycerides greater than or equal to 1.7 mmol/l or HDL cholesterol less than 0.9 mmol in men and less then 1.0 mmol in women, blood pressure greater than or equal to 140/90 mmHg, urinary albumin excretion rate greater than 20 μg/min or albumin-to-creatinine ratio greater than or equal to 30 mg/g (Diabetes Care, 2005, 28(9): 2289-2304).

A statement from the American Diabetes Association and the European Association for the Study of Diabetes comments on the construct of metabolic syndrome to denote risk factor clustering. In addition to suggestions for research of the underlying pathophysiology, the recommendations include individually and aggressively treating all cardiovascular disease risk factors (Diabetes Care, 2005, 28(9): 2289-2304). Therefore, another embodiment of the present invention is a method of treating cardiovascular disease risk factors with ISIS 113715. Also contemplated is the use of ISIS 113715 to treat a subject having waist circumference of greater than 102 cm for men or greater than 88 cm for women, triglyceride levels greater than or equal to 150 mg/dL, HDL cholesterol levels of less than 40 mg/dL for men and less than 50 mg/dL for women, blood pressure greater than or equal to 130/85 mm Hg, or fasting glucose levels greater than or equal to 110 mg/dL, or any combination thereof. Also contemplated is a method of lowering HbA_{1c} levels or fasting glucose levels in a subject having waist circumference of greater than 102 cm for men or greater than 88 cm for women, triglyceride levels greater than or equal to 150 mg/dL, HDL cholesterol levels of less than 40 mg/dL for men and less than 50 mg/dL for women, blood pressure greater than or equal to 130/85 mm Hg, or fasting glucose levels greater than or equal to 110 mg/dL, or any combination thereof by administering ISIS 113715. Also contemplated is a method of altering lipid profile, increasing adiponectin levels, or decreasing apolipoprotein B levels in such a subject. Also contemplated is the use of ISIS 113715 to treat a subject having diabetes, impaired fasting glucose, impaired glucose tolerance, or insulin resistance (assessed by clamp studies), waist-to-hip ratio greater than 0.90 in men or greater than 0.85 in women, serum triglycerides greater than or equal to 1.7 mmol/l or HDL cholesterol less than 0.9 mmol in men and less then 1.0 mmol in women, blood pressure greater than or equal to 140/90 mmHg, urinary albumin excretion rate greater than 20 μg/min, or albumin-to-creatinine ratio greater than or equal to 30 mg/g, or a combination thereof. Also contemplated is a method of altering lipid profile, increasing adiponectin levels, or decreasing apolipoprotein B levels in such a subject.

### Cardiovascular Risk Factors

Conditions associated with risk of developing a cardiovascular disease include, but are not limited to, history of myocardial infarction, unstable angina, stable angina, coronary artery procedures (angioplasty or bypass surgery), evidence of clinically significant myocardial ischemia, noncoronary forms of atherosclerotic disease (peripheral arterial disease, abdominal aortic aneurysm, carotid artery disease), diabetes, cigarette smoking, hypertension, low HDL cholesterol, family history of premature CHD, obesity, physical inactivity, elevated triglyceride, or metabolic syndrome (Jama, 2001, 285, 2486-2497; Grundy et al., Circulation, 2004, 110, 227-239).

### Salts, Prodrugs and Bioequivalents

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides of the invention are prepared as SATE ((S-acetyl-2-thioethyl) phosphate) derivatives according to the methods described in International Patent Application Publication No. WO 93/24510, published Dec. 9, 1993; and International Patent Application Publication No. WO 94/26764, and U.S. Pat. No. 5,770,713.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic and inorganic acid salts of the amines. Acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoc acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts of antisense oligonucleotides are useful and are well accepted for therapeutic administration to humans. In another embodiment, sodium salts of dsRNA compounds are also provided.

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Active Components for Combination with PTP1B Antisense Compounds in Compositions or in Therapy

### A. Glucose-Lowering Drugs/Agents/Therapeutics, Anti-Obesity Drugs/Agents/Therapeutics, Lipid-Lowering Drugs/Agents/Therapeutics

Compounds of the invention, particularly ISIS 113715, may be used in combination therapies, wherein an additive effect is achieved by administering one or more compounds of the invention and one or more other suitable therapeutic/prophylactic compounds to treat a condition. Suitable therapeutic/prophylactic compound(s) include, but are not limited to, glucose-lowering agents (also referred to herein as glucose-lowering drugs or glucose-lowering therapeutics), anti-obesity agents (also referred to herein as anti-obesity drugs or anti-obesity therapeutics), and lipid lowering agents (also referred to herein as lipid-lowering drugs or lipid-lowering therapeutics). Glucose lowering agents include, but are not limited to, PPAR agonists, dipeptidyl peptidase (IV) inhibitors, GLP-1 analogs, insulin or insulin analogs, insulin secretagogues, SGLT2 inhibitors, human amylin analogs, biguanides, or alpha-glucosidase inhibitors. Glucose lowering agents include, but are not limited to hormones, hormone mimetics, or incretin mimetics (e.g., insulin, including inhaled insulin, GLP-1 or GLP-1 analogs such as liraglutide, or exenatide), DPP(IV) inhibitors, a sulfonylurea (e.g., acetohexamide, chlorpropamide, tolbutamide, tolazamide, glimepiride, a glipizide, glyburide or a gliclazide), a biguanide (metformin), a meglitinide (e.g., nateglinide or repaglinide), a thiazolidinedione or other PPAR-gamma agonists (e.g., pioglitazone or rosiglitazone) an alpha-glucosidase inhibitor (e.g., acarbose or miglitol), or an antisense compound not targeted to PTP1B. Also included are dual PPAR-agonists (e.g., muraglitazar, being developed by Bristol-Myers Squibb, or tesaglitazar, being developed by Astra-Zeneca). Also included are other diabetes treatments in development (e.g. LAF237, being developed by Novartis; MK-0431, being developed by Merck; or rimonabant, being developed by Sanofi-Aventis). Also included are GLP-1 mimetics in development, including, but not limited to, those being developed by Roche, ConjuChem, Sanofi-Aventis, Teijin Pharma Limited, Ipsen Pharmaceuticals, and Servier Research Institute. Also included are SGLT2 inhibitors in development, including, but not limited to, those being developed by Glaxo Smith Kline or AVE2268 in development at Sanofi-Aventis. Also included are DPP(IV) inhibitors in development, including, but not limited to, those being developed by Novartis (e.g. vildagliptin), Merck, GSK, or BMS. Also included are glucokinase inhibitors in development. Anti-obesity agents include, but are not limited to, appetite suppressants (e.g. phentermine or Meridia™), fat absorption inhibitors such as orlistat (e.g. Xenical™), and modified forms of ciliary neurotrophic factor which inhibit hunger signals that stimulate appetite. Anti-obesity agents include peripheral or CNS-based agents. Lipid lowering agents include, but are not limited to, bile salt sequestering resins (e.g., cholestyramine, colestipol, and colesevelam hydrochloride), HMGCoA-reductase inhibitors (e.g., lovastatin, pravastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), CETP inhibitors (e.g. torcetrapib, and JTT-705) MTP inhibitors (e.g., implitapide), inhibitors of bile acid transporters (apical sodium-dependent bile acid transporters), regulators of hepatic CYP7a, ACAT inhibitors (e.g. Avasimibe), estrogen replacement therapeutics (e.g., tamoxigen), synthetic HDL (e.g. ETC-216), anti-inflammatories (e.g., glucocorticoids), or an antisense compound not targeted to PTP1B. One or more of these drugs may be combined with one or more of the antisense inhibitors of PTP1B to achieve an additive therapeutic effect.

Diabetes agents, including insulin, other hormones and hormone analogs and mimetics, and other glucose lowering agents, including orally administered glucose lowering drugs, may also be combined with antisense inhibitors of PTP1B. The term “glucose-lowering agent” includes, but is not limited to, the sulfonylureas, biguanides, meglitinides, peroxisome proliferator-activated receptor-gamma (PPAR-gamma) agonists (e.g., thiazolidinediones) and alpha-glucosidase inhibitors.

Sulfonylureas work by stimulating beta-cell insulin secretion in the pancreas, and may also improve insulin sensitivity in peripheral tissues. Early sulfonylureas such as acetohexamide (Dymelor™), chlorpropamide (Diabinese™, Glucamide™), tolbutamide (Orinase™, Mobenol™), and tolazamide (Tolamide™, Tolinase™) have been generally replaced with newer sulfonureas with better side-effect profiles (specifically lower cardiovascular risk), such as glimepiride (Amaryl™), glipizide (Glucotrol™), glipizide extended release (Glucotrol XL™), glyburide (Micronase™, Euglucol™, Diabeta™), gliclazide (Diamicron™, and micronized glyburide (Glynase™) (Luna & Feinglos; AACE et al., 2002). Side effects of sulfonylureas include hypoglycemia and weight gain.

Biguanides such as Metformin (Glucophage™) work by decreasing hepatic glucose output and enhancing insulin sensitivity in hepatic and peripheral tissues. Metformin is contrainidated in patients with congestive heart failure or severe liver disease.

Meglitinides work by stimulating the beta cells in the pancreas to produce insulin. Nateglinide (Starlix™) and repaglinide (Prandin™) are examples of this class.

Peroxisome proliferator-activated receptor-gamma (PPAR-gamma) agonists such as the thiazolidinediones enhance insulin sensitivity in muscle and adipose tissue and, to a lesser extent, inhibit hepatic glucose production. Thiazolidinediones include pioglitazone (Actos™) and rosiglitazone (Avandia™; GlaxoSmithKline). The first thiazolidinedione approved for use in the United States, troglitazone (Rezulin™), was withdrawn from the market because of severe liver toxicity. Thiazolidinediones also affect the lipid profiles of patients with type 2 diabetes. Studies have shown that rosiglitazone is associated with increases in total, LDL, and HDL cholesterol levels, and either no changes or increases in triglyceride levels. Pioglitazone has been associated with mean decreases in triglyceride levels, mean increases in HDL cholesterol levels, and no consistent mean changes in LDL and total cholesterol levels. Other potential side effects associated with thiazolidinediones include weight gain, slow onset of action, and potential liver toxicity (Luna & Feinglos, 2001).

New PPAR-gamma agonists are being developed; these include isaglitazone (netoglitazone) and the dual-acting PPAR agonists which have affinities for both PPAR-gamma and PPAR-alpha. Examples of dual-acting PPAR agonists are BMS-298585 and tesaglitazar. Agonists of other PPARs (e.g., alpha, delta) or pan-PPAR agonists may also be useful.

Alpha-glucosidase inhibitors inhibit an enzyme found in the lining of the small intestine that is responsible for the breakdown of complex carbohydrates before they are absorbed. Such inhibitors include acarbose (Precose™) and miglitol (Glyset™).

Oral glucose-lowering drugs are often used in combination to treat Type 2 diabetes. While many combinations of the above are possible, several are already marketed as a combined formulation (for example, Avandamet™ (Rosiglitazone+Metformin); Glucovance™ (glyburide/metformin); and Metaglip™ (glipizide/metformin). These and other combined formulations for treatment of diabetes or obesity may be administered in combination with antisense inhibitors of PTP1B.

Other classes of glucose-lowering, diabetes drugs are being developed. As alternatives to regular insulin, which is administered by injection, insulin analogs such as insulin lispro (Humalog™) and insulin glargine (Lantus™) may be used. Both are given by injection as is regular insulin, but result in fewer hypoglycemic events than regular insulin. In addition the onset and duration of action with these is different from regular insulin. A follow-up analog to insulin glargine, insulin glulisine, is being developed by Aventis. Novo Nordisk is developing insulin detemir, a long-acting analog.

Alternative formulations/delivery methods for regular insulin are also being developed. Both liquid and dry powder inhaled insulin formulations are currently in late-stage development or have been recently approved—examples include recently approved Exubera™ (Nektar/Pfizer/Aventis), which is a powder, and AERx™ (Aradigm/Novo Nordisk), which is an aerosolized liquid. While inhaled insulin is expected to be viewed as more convenient and less invasive than injected insulin, the cost is expected to be much greater for inhaled insulin.

Several companies are developing oral formulations of insulin. Oralin™ (Generex Biotechnology) is the farthest along in development but there are others.

Other hormones and hormone mimetics being developed include pramlintide acetate (Symlin™), and GLP-1. GLP-1 receptor agonists and GLP-1 analogs are being evaluated for clinical use as antidiabetic agents. GLP-1 itself has a short half-life due to N-terminal degradation of the peptide by Dipeptidyl Peptidase (DPP-IV)-mediated cleavage at the position 2 alanine. This limits the clinical usefulness of native GLP-1 or synthetic versions thereof. Longer acting analogs have been developed, including Exendin-4 (Exenatide™, Exenatide LAR™), a DP IV-resistant GLP-1 analog and Liraglutide™, an acylated albumin-bound human GLP-1 analog.

DPP-UV inhibitors are also being explored as drugs and one (LAF-237, Novartis) is currently in advanced clinical trials. Glucagon inhibitors may also be useful for diabetes.

Other peptides such as pituitary adenylate cyclase-activating polypeptide (PACAP) and Peptide YY (PYY) (and its subpeptide PYY[3-36]) are also under study for diabetes and/or obesity (Yamamoto et al., 2003, Diabetes 52, 1155-1162; Pittner et al., Int. J. Obes. Relat. Metab. Disord. 2004, 28, 963-71).

Any of these glucose-lowering drugs is useful in combination with ISIS 113715 or another antisense inhibitor of PTP1B as described herein. One or more of these drugs may be combined in a single composition with one or more of the antisense inhibitors or PTP 1B, or used in therapies for combined administration, i.e., sequential or concurrent administration thereof.

Antisense inhibition of PTP 1B is shown hereinbelow to reduce weight gain of animals on high-fat diets and may be useful in treatment of obesity. The use of weight loss agents has also been considered useful in diabetes management in general and for delaying or preventing the development or progression of frank Type 2 diabetes in patients with impaired glucose tolerance (Heymsfield S B, 2000, Archives of Internal Medicine, 160, 1321-1326). Thus, anti-obesity drugs are useful in combination with antisense inhibitors of PTP1B expression in pharmaceutical compositions or in combined therapeutic regimens. Examples of anti-obesity drugs (also called “diet drugs”) include, without limitation, appetite suppressants such as phentermine and Meridia™, fat absorption inhibitors such as orlistat (Xenical™), and Axokine™, a modified form of ciliary neurotrophic factor, which inhibits hunger signals that stimulate appetite. Other drugs or classes of drugs under evaluation for obesity are CB1 inverse agonists, PYY, MCH4 and MTP inhibitors.

Any of the aforementioned is useful in combination with ISIS 113715 or another antisense inhibitor of PTP1B according to this invention. Combined compounds (two or more) may be used together or sequentially.

B. Drugs/Other Antisense Compounds Directed to Other Cellular Targets in Combination with PTP1B Antisense Compounds

In another related embodiment, compositions of the invention may contain one or more antisense compounds, particularly oligonucleotides, targeted to PTP1B, and one or more additional antisense compounds targeted to a second nucleic acid target. A variety of useful targets for such antisense compounds are listed below and known in the art. Two or more combined compounds may be used together or sequentially in a composition or in a combined therapeutic regimen.

Thus, also advantageous for combination with antisense inhibitors of PTP1B include: inhibitors of genes or gene products implicated in glucose and/or insulin metabolism, lipid and/or triglyceride levels, or obesity. These inhibitors may include but are not limited to small molecules, antibodies, peptide fragments or antisense inhibitors (including ribozymes and siRNA molecules). Antisense inhibitors are particularly suitable.

Examples of genes to be inhibited include glucagon receptor, glucocorticoid receptor, 26-HSD, hydroxysteroid 11-beta dehydrogenase 1, Forkhead O1A, other forkhead genes, fructose 1,6-bisphosphatase, glucose-6-phosphatase (translocase and/or catalytic subunits), diacylglycerol acyltransferase (DGAT1), diacylglycerol acyltransferase-2 (DGAT2), stearoyl CoA desaturase 1 (SCD-1), Acetyl CoA Carboxylase 1 and 2, hormone sensitive lipase, fatty acid synthase, sodium-glucose cotransporters 1 and 2 (SGLT 1 and 2), Microsomal triglyceride transfer protein (MTP), apolipoprotein-CIII, apoliprotein B (particularly ApoB100) and other genes whose inhibitors are believed to cause glucose, cholesterol and/or triglyceride lowering or to combat obesity. Antisense compounds inhibiting expression of some of these targets are also likely to be categorized as glucose-lowering drugs.

### C. Dosing and Administration of ISIS 113715 as Monotherapy or in Combination Compositions

As used herein, a “dose” refers to the amount of drug given to a human subject in one day; e.g. by intravenous or subcutaneous administration, in a single administration or divided into multiple administrations.

The preferred range of doses of ISIS 113715 is from about 50 to about 900 mg. It is understood that doses of 50, 100, 150, 200, 250, 300, 350, 400, 450, 500, 550, 600, 650, 700, 750, 800, 850, or 900 mg per week all fall within the range of 50-900 mg. As used herein, the terms “patient” and “subject” are interchangeable.

A preferred dose range is about 0.5 to about 7.5 mg/kg of body weight per week or the equivalent. Another preferred dose range is about 0.25 mg/kg to about 9 mg/kg per week or the equivalent. Another preferred dose range is about 1 to about 6 mg/kg per week or the equivalent. Additional ranges include about 0.1-5 mg/kg, about 0.5-3 mg/kg, about 0.5-8 mg/kg, about 0.25-3 mg/kg, about 5-9 mg/kg, about 7-9 mg/kg, about 3-5 mg/kg, or about 0.25-2 mg/kg.

Dosing regimens may include doses during a loading period and/or a maintenance period. During the loading period, which usually or most often occurs at the initiation of therapy and which lasts approximately one to three weeks (although it could be more or less, e.g. 3, 4, 5, 6, or 22, 23, 24, 25 days), a single administration may be given or multiple administrations may be given every day, every 2 days, every 3 days, every 4 days, every 5 days, every 6 days, or every week. Alternatively, the loading period may last about 28 days, although it could be more or less, e.g., 11, 12, 13, 14, 15, 16, 17, 18, 19, 20, 21, 22, 23, 24, 25, 26, 27, 28, 29, 30, 31, 32, 33, 34 or 35 days, and a single administration may be given every day, every 2 days, every 3 days, every 4 days, every 5 days, every 6 days, or every 7 days. During a maintenance period, which follows the loading period and may last for a number of years or the duration of the lifetime of the subject, doses may be given at a frequency ranging from every day to every 3 months, which is understood to include every day, every 2 days, every 3 days, every 4 days, every 5 days, every 6 days, every week, every 2 weeks, every 3 weeks, every 4 weeks, every month, every 2 months, or every 3 months.

An alternative-dosing regimen may include doses administered during a maintenance period, without a preceding loading period. Doses may be given at a frequency ranging from every day to every three months, which is understood to include every day, every 2 days, every 3 days, every 4 days, every 5 days, every 6 days, every week, every 2 weeks, every 3 weeks, every 4 weeks, every month, every 2 months, or every 3 months.

In one embodiment, the loading phase is comprised of 3 doses each of about 0.5 mg/kg to about 7.5 mg/kg which are administered over about one week. In another embodiment, the loading phase is comprised of 4 doses of about 0.5 mg/kg to about 7.5 mg/kg which are administered over about two weeks.

In another embodiment, the loading phase is comprised of 5 doses of about 0.5 to about 7.5 mg/kg which are administered over about three weeks. In one embodiment, a loading phase is followed by a maintenance phase during which a dose equivalent to about 0.5 to about 7.5 mg/kg per week is administered about once per week, about once every two weeks, or about once per month. In one embodiment, doses are administered for either the loading period or the maintenance period or both subcutaneously or intravenously. Administration need not be by the same route for loading and maintenance.

### D. Bioavailability

The term “bioavailability” refers to a measurement of that portion of an administered drug which reaches the circulatory system (e.g. blood, especially blood plasma) when a particular mode of administration is used to deliver the drug. For example, when a subcutaneous mode of administration is used to introduce the drug into a human subject, the bioavailability for that mode of administration may be compared to a different mode of administration (e.g. an intravenous mode of administration) and extrapolations made to facilitate determination of the proper therapy. In general, bioavailability can be assessed by measuring the area under the curve (AUC) or the maximum serum or plasma concentration (Cmax) of the unchanged form of a drug following administration of the drug to a human subject. AUC is a determination of the Area Under the Curve plotting the serum or plasma concentration of a drug along the ordinate (Y-axis) against time along the abscissa (X-axis). Generally, the AUC for a particular drug can be calculated using methods known to those of ordinary skill in the art and as described in G. S. Banker, Modern Pharmaceutics, Drugs and the Pharmaceutical Sciences, 4th Ed, (May 2002). In some embodiments, the area under a drug's blood plasma concentration curve (AUCsc) after subcutaneous administration may be divided by the area under the drug's plasma concentration curve after intravenous administration (AUCiv) to provide a dimensionless quotient (relative bioavailability, RB) that represents fraction of drug absorbed via the subcutaneous route as compared to the intravenous route.

Oligonucleotide concentrations in plasma may be determined by methods routine in the art, for example, by hybridization-based ELISA.

While the present invention has been described with specificity in accordance with certain of its preferred embodiments, the following examples serve only to illustrate the invention and are not intended to limit the same.

### EXAMPLES

### Example 1

### Nucleoside Phosphoramidites for Oligonucleotide Synthesis Deoxy and 2′-alkoxy amidites

2′-Deoxy and 2′-methoxy beta-cyanoethyl-diisopropyl phosphoramidites were purchased from commercial sources (e.g. Chemgenes, Needham, Mass. or Glen Research, Inc., Sterling, Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides was utilized, except the wait step after pulse delivery of tetrazole and base was increased to 360 seconds.

Oligonucleotides containing 5-methyl-2′-deoxycytidine (5-Me-C) nucleotides were synthesized according to published methods [Sanghvi, et. al., Nucleic Acids Research, 1993, 21, 3197-3203] using commercially available phosphoramidites (Glen Research, Sterling, Va., or ChemGenes, Needham, Mass.).

### 2′-O-(2-Methoxyethyl) modified amidites

2′-O-Methoxyethyl-substituted nucleoside amidites are prepared as follows, or alternatively, as per the methods of Martin, P., Helvetica Chimica Acta, 1995, 78, 486-504.

### 2,2′-Anhydro [1-(beta-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid that was crushed to a light tan powder (57 g, 85% crude yield). The NMR spectrum was consistent with the structure, contaminated with phenol as its sodium salt (ca. 5%). The material was used as is for further reactions (or it can be purified further by column chromatography using a gradient of methanol in ethyl acetate (10-25%) to give a white solid, mp 222-4° C.).

### 2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product. Additional material was obtained by reworking impure fractions.

### 2′-O-Methoxyethyl-5′-O-dimethoxytrityl-5-methyluridine

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/hexane/acetone (5:5:1) containing 0.5% Et₃NH. The pure fractions were evaporated to give 164 g of product. Approximately 20 g additional was obtained from the impure fractions to give a total yield of 183 g (57%).

### 3′-O-Acetyl-2′-O-methoxyethyl-5′-O-dimethoxytrityl-5-methyluridine

2′-O-Methoxyethyl-5′-O-dimethoxytrityl-5-methyl-uridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by TLC by first quenching the TLC sample with the addition of MeOH. Upon completion of the reaction, as judged by TLC, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/hexane (4:1). Pure product fractions were evaporated to yield 96 g (84%). An additional 1.5 g was recovered from later fractions.

### 3′-O-Acetyl-2′-O-methoxyethyl-5′-O-dimethoxytrityl-5-methyl-4-triazoleuridine

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 h using an overhead stirrer. POCl₃ was added dropwise, over a 30 minute period, to the stirred solution maintained at 0-1° C., and the resulting mixture stirred for an additional 2 hours. The first solution was added dropwise, over a 45 minute period, to the latter solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

### 2′-O-Methoxyethyl-5′-O-dimethoxytrityl-5-methyl cytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (TLC showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

### N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, TLC showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/hexane (1:1) containing 0.5% Et₃NH as the eluting solvent. The pure product fractions were evaporated to give 90 g (90%) of the title compound.

### N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) was dissolved in CH₂Cl₂ (1 L). Tetrazole diisopropylamine (7.1 g) and 2-cyanoethoxytetra(isopropyl)phosphite (40.5 mL, 0.123 M) were added with stirring, under a nitrogen atmosphere. The resulting mixture was stirred for 20 hours at room temperature (TLC showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAc/hexane (3:1) as the eluting solvent. The pure fractions were combined to give 90.6 g (87%) of the title compound.

### Example 2

### Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 h), the oligonucleotides were purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published International Patent Application Publication Nos. WO 94/17093 and WO 94/02499, herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample was again lyophilized to dryness. The pellet is resuspended in 1 M TBAF in THF for 24 hrs at room temperature to deprotect the 2′ positions. The reaction is then quenched with 1 M TEAA and the sample is then reduced to ½ volume by rotovac before being desalted on a G25 size exclusion column. The oligo recovered is then analyzed spectrophoto-metrically for yield and for purity by capillary electrophoresis and by mass spectrometry.

### [2′-O-(2-Methoxyethyl)]-[2′-deoxy]-[2′-O-(Meth-oxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]-[2′-deoxy Phosphorothioate]-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 4

### Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 5

### Oligonucleotide Synthesis—96 Well Plate Format

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected beta-cyanoethyldiiso-propyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 6

### Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 7

### Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

### T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 μgs per mL (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

### A549 Cells:

The human lung carcinoma cell line A549 was obtained from the American Type Culture Collection (ATCC) (Manassas, Va.). A549 cells were routinely cultured in DMEM basal media (Gibco/Life Technologies, Gaithersburg, Md.) supplemented with 10% fetal calf serum (Gibco/Life Technologies, Gaithersburg, Md.), penicillin 100 units per ML, and streptomycin 100 μg per mL (Gibco/Life Technologies, Gaithersburg, Md.). Cells were routinely passaged by trypsinization and dilution when they reached 90% confluence.

### NHDF Cells:

Human neonatal dermal fibroblasts (NHDF) were obtained from the Clonetics Corporation (Walkersville Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

### HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville Md.) formulated as recommended by the supplier. Cells were routinely maintained for up to 10 passages as recommended by the supplier.

### PC-12 Cells:

The rat neuronal cell line PC-12 was obtained from the American Type Culture Collection (Manassas, Va.). PC-12 cells were routinely cultured in DMEM, high glucose (Gibco/Life Technologies, Gaithersburg, Md.) supplemented with 10% horse serum+5% fetal calf serum (Gibco/Life Technologies, Gaithersburg, Md.). Cells were routinely passaged by trypsinization and dilution when they reached 90% confluence. Cells were seeded into 96-well plates (Falcon-Primaria #3872) at a density of 20000 cells/well for use in RT-PCR analysis.

For Northern blotting or other analysis, cells may be seeded onto 100 mm or other standard tissue culture plates and treated similarly, using appropriate volumes of medium and oligonucleotide.

Treatment with Antisense Compounds:

When cells reached 80% confluency, they were treated with oligonucleotide. For cells grown in 96-well plates, wells were washed once with 200 μL OPTI-MEM™-1 reduced-serum medium (Gibco BRL) and then treated with 130 μL of OPTI-MEM™-1 medium containing 3.75 μg/mL LIPOFECTIN™ reagent (Gibco BRL) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control, oligonucleotide at a range of concentrations. For human cells the positive control oligonucleotide is ISIS 13920, TCCGTCATCGCTCCTCAGGG, SEQ ID NO: 1, a 2′-O-methoxyethyl gapmer (2′-O-methoxyethyls shown in bold) with a phosphorothioate backbone which is targeted to human H-ras. For mouse or rat cells the positive control oligonucleotide is ISIS 15770, ATGCATTCTGCCCCCAAGGA, SEQ ID NO: 2, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-Ha-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments.

### Example 8

### Analysis of Oligonucleotide Inhibition of PTP1B Expression

Antisense modulation of PTP1B expression can be assayed in a variety of ways known in the art. For example, PTP1B mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions. Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing).

Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed as multiplexable. Other methods of PCR are also known in the art.

Protein levels of PTP1B can be quantitated in a variety of ways well known in the art, such as immunoprecipitation, Western blot analysis (immunoblotting), ELISA or fluorescence-activated cell sorting (FACS). Antibodies directed to PTP1B can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 9

### Poly(A)+ mRNA Isolation

Poly(A)+ mRNA was isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C. was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 10

### Total RNA Isolation

Total mRNA was isolated using an RNEASY 96™ kit and buffers purchased from Qiagen, Inc. (Valencia, Calif.) following the manufacturer's recommended procedures. Briefly, for cells grown on 96-well plates, growth medium was removed from the cells and each well was washed with 200 μL cold PBS. 100 μL Buffer RLT was added to each well and the plate vigorously agitated for 20 seconds. 100 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 15 seconds. 1 mL of Buffer RW1 was added to each well of the RNEASY 96™ plate and the vacuum again applied for 15 seconds. 1 mL of Buffer RPE was then added to each well of the RNEASY 96™ plate and the vacuum applied for a period of 15 seconds. The Buffer RPE wash was then repeated and the vacuum was applied for an additional 10 minutes. The plate was then removed from the QIAVAC™ manifold and blotted dry on paper towels. The plate was then re-attached to the QIAVAC™ manifold fitted with a collection tube rack containing 1.2 mL collection tubes.

RNA was then eluted by pipetting 60 μL water into each well, incubating 1 minute, and then applying the vacuum for 30 seconds. The elution step was repeated with an additional 60 μL water.

The repetitive pipetting and elution steps may be automated using a QIAGEN Bio-Robot™ 9604 (Qiagen, Inc., Valencia, Calif.). Essentially, after lysing of the cells on the culture plate, the plate is transferred to the robot deck where the pipetting, DNase treatment and elution steps are carried out.

### Example 11

### Real-Time Quantitative PCR Analysis of PTP1B mRNA Levels

Quantitation of PTP1B mRNA levels was determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

PCR reagents were obtained from PE-Applied Biosystems, Foster City, Calif. RT-PCR reactions were carried out by adding 25 μL PCR cocktail (1×TAQMAN™ buffer A, 5.5 mM MgCl₂, 300 μM each of dATP, dCTP and dGTP, 600 μM of dUTP, 1100 nM each of forward primer, reverse primer, and probe, 20 Units RNase inhibitor, 1.25 Units AMPLITAQ GOLD™ reagent, and 12.5 Units MuLV reverse transcriptase) to 96 well plates containing 25 μL poly(A) mRNA solution. The RT reaction was carried out by incubation for 30 minutes at 48° C. Following a 10 minute incubation at 95° C. to activate the AMPLITAQ GOLD™ reagent, 40 cycles of a two-step PCR protocol were carried out: 95° C. for 15 seconds (denaturation) followed by 60° C. for 1.5 minutes (annealing/extension).

Probes and primers to human PTP1B were designed to hybridize to a human PTP1B sequence, using published sequence information (Genank® accession number M31724, incorporated herein as SEQ ID NO: 3). For human PTP1B the PCR primers were:

forward primer: GGAGTTCGAGCAGATCGACAA (SEQ ID NO: 4)
reverse primer: GGCCACTCTACATGGGAAGTC (SEQ ID NO: 5) and the PCR probe was:

### FAM-AGCTGGGCGGCCATTTACCAGGAT-TAMRA

(SEQ ID NO: 6) where FAM (PE-Applied Biosystems, Foster City, Calif.) is the fluorescent reporter dye) and TAMRA (PE-Applied Biosystems, Foster City, Calif.) is the quencher dye. For human GAPDH the PCR primers were:
forward primer: GAAGGTGAAGGTCGGAGTC (SEQ ID NO: 7)
reverse primer: GAAGATGGTGATGGGATTTC (SEQ ID NO: 8) and the PCR probe was: 5′JOE-CAAGCTTCCCGTTCTCAGCC-TAMRA 3′ (SEQ ID NO: 9) where JOE (PE-Applied Biosystems, Foster City, Calif.) is the fluorescent reporter dye) and TAMRA (PE-Applied Biosystems, Foster City, Calif.) is the quencher dye.

Probes and primers to rat PTP1B were designed to hybridize to a rat PTP1B sequence, using published sequence information (GenBank® accession number M33962, incorporated herein as SEQ ID NO:10). For rat PTP1B the PCR primers were:

forward primer: CGAGGGTGCAAAGTTCATCAT (SEQ ID NO:111)
reverse primer: CCAGGTCTTCATGGGAAAGCT (SEQ ID NO: 12) and the PCR probe was:

### FAM-CGACTCGTCAGTGCAGGATCAGTGGA-TAMRA

(SEQ ID NO: 13) where FAM (PE-Applied Biosystems, Foster City, Calif.) is the fluorescent reporter dye) and TAMRA (PE-Applied Biosystems, Foster City, Calif.) is the quencher dye. For rat GAPDH the PCR primers were:
forward primer: TGTTCTAGAGACAGCCGCATCTT (SEQ ID NO: 14)
reverse primer: CACCGACCTTCACCATCTTGT (SEQ ID NO: 15) and the PCR probe was: 5′JOE-TTGTGCAGTGCCAGCCTCGTCTCA-TAMRA 3′ (SEQ ID NO: 16) where JOE (PE-Applied Biosystems, Foster City, Calif.) is the fluorescent reporter dye) and TAMRA (PE-Applied Biosystems, Foster City, Calif.) is the quencher dye.

### Example 12

### Northern Blot Analysis of PTP1B mRNA Levels

Eighteen hours after antisense treatment, cell monolayers were washed twice with cold PBS and lysed in 1 mL RNAZOL™ reagent (TEL-TEST “B” Inc., Friendswood, Tex.). Total RNA was prepared following manufacturer's recommended protocols. Twenty μgs of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™UV Crosslinker 2400 (Stratagene, Inc, La Jolla, Calif.) and then robed using QUICKHYB™ hybridization solution (Stratagene, La Jolla, Calif.) using manufacturer's recommendations for stringent conditions.

To detect human PTP1B, a human PTP1B specific probe was prepared by PCR using the forward primer GGAGTTCGAGCAGATCGACAA (SEQ ID NO: 4) and the reverse primer GGCCACTCTACATGGGAAGTC (SEQ ID NO: 5). To normalize for variations in loading and transfer efficiency membranes were stripped and probed for human glyceraldehyde-3-phosphate dehydrogenase (GAPDH) RNA (Clontech, Palo Alto, Calif.).

To detect rat PTP1B, a rat PTP1B specific probe was prepared by PCR using the forward primer CGAGGGTGCAAAGTTCATCAT (SEQ ID NO:11) and the reverse primer CCAGGTCTTCATGGGAAAGCT (SEQ ID NO: 12). To normalize for variations in loading and transfer efficiency membranes were stripped and probed for rat glyceraldehyde-3-phosphate dehydrogenase (GAPDH) RNA (Clontech, Palo Alto, Calif.).

Hybridized membranes were visualized and quantitated using a PHOSPHORIMAGER™ apparatus and IMAGEQUANT™ Software V3.3 (Molecular Dynamics, Sunnyvale, Calif.). Data was normalized to GAPDH levels in untreated controls.

### Example 13

### Effects of ISIS 113715 on PTP1B Levels In Vitro

ISIS 113715 is an oligonucleotide having the sequence “GCTCCTTCCACTGATCCTGC” (incorporated herein as SEQ ID NO: 17), having a ten-deoxynucleotide gap region flanked on its 3′ and 5′ ends with five 2′-O-(2-methoxyethyl) nucleotides, wherein all the cytosines are 5-methylcytosines, and all of the internucleoside linkages are phosphorothioate linkages. The binding site for ISIS 113715 is within the coding region of the PTP-1B mRNA. The binding site of ISIS 113715 is conserved across all species studied and the drug is active in all species studied to date including mouse, rat, dog, monkey, and man. Several experiments were conducted to evaluate the potency of ISIS 113715 in human cell lines (T24 and HepG2). ISIS 113715 was found to be a very potent inhibitor of human PTP1B, with IC₅₀s between 50-150 nM.

### Example 14

### Effects of Antisense Inhibition of PTP1B (ISIS 113715) on Blood Glucose Levels

db/db mice are used as a model of Type 2 diabetes. These mice are hyperglycemic, obese, hyperlipidemic, and insulin resistant. The db/db phenotype is due to a mutation in the leptin receptor on a C57BLKS background. However, a mutation in the leptin gene on a different mouse background can produce obesity without diabetes (ob/ob mice). Leptin is a hormone produced by fat that regulates appetite and animals or humans with leptin deficiencies become obese. Heterozygous db/wt mice (known as lean littermates) do not display the hyperglycemia/hyperlipidemia or obesity phenotype and are used as controls.

In accordance with the present invention, ISIS 113715 (GCTCCTTCCACTGATCCTGC, SEQ ID NO: 17) was investigated in experiments designed to address the role of PTP1B in glucose metabolism and homeostasis. ISIS 113715 is completely complementary to and is targeted to sequences in the coding region of the human PTP1B nucleotide sequence incorporated herein as SEQ ID NO: 3 (starting at nucleotide 951 of human PTP1B; GenBank® Accession No. M31724), of the rat PTP1B nucleotide sequence incorporated herein as SEQ ID NO: 10 (starting at nucleotide 980 of rat PTP1B; GenBank® Accession No. M33962) and of the mouse PTP1B nucleotide sequence incorporated herein as SEQ ID NO: 18 (starting at nucleotide 1570 of mouse PTP1B; GenBank® Accession No. U24700). The control used is ISIS 29848 (NNNNNNNNNNNNNNNNNNNN, SEQ ID NO: 19) where N is a mixture of A, G, T and C.

Male db/db mice and lean (heterozygous, i.e., db/wt) littermates (age 9 weeks at time 0) were divided into matched groups (n=6) with the same average blood glucose levels and treated by intraperitoneal injection once a week with saline, ISIS 29848 (the control oligonucleotide) or ISIS 113715. db/db mice were treated at a dose of 10, 25 or 50 mg/kg of ISIS 113715 or 50 mg/kg of ISIS 29848 while lean littermates were treated at a dose of 50 or 100 mg/kg of ISIS 113715 or 100 mg/kg of ISIS 29848. Treatment was continued for 4 weeks with blood glucose levels being measured on day 0, 7, 14, 21 and 28 (Ascensia Elite™ glucometer, Bayer, Tarrytown N.Y.).

By day 28 in db/db mice, blood glucose levels were reduced at all doses from a starting level of 300 mg/dL to 225 mg/dL for the 10 mg/kg dose, 175 mg/dL for the 25 mg/kg dose and 125 mg/dL for the 50 mg/kg dose. These final levels are within normal range for wild-type mice (170 mg/dL). The mismatch control and saline treated levels were 320 mg/dL and 370 mg/dL at day 28, respectively.

In lean littermates, blood glucose levels remained constant throughout the study for all treatment groups (average 120 mg/dL). These results indicate that treatment with ISIS 113715 reduces blood glucose in db/db mice and that there is no hypoglycemia induced in the db/db or the lean littermate mice as a result of the oligonucleotide treatment.

In a similar experiment, ob/ob mice and their lean littermates (heterozygous, i.e., ob/wt) were dosed twice a week at 50 mg/kg with ISIS 113715, ISIS 29848 or saline control and blood glucose levels were measured at the end of day 7, 14 and 21. Treatment of ob/ob mice with ISIS 113715 resulted in the largest decrease in blood glucose over time going from 225 mg/dL at day 7 to 95 mg/dL at day 21. Ob/ob mice displayed an increase in plasma glucose over time from 300 mg/dL to 325 mg/dL while treatment with the control oligonucleotide reduced plasma glucose from an average of 280 mg/dL to 130 mg/dL. In the lean littermates plasma glucose levels remained unchanged in all treatment groups (average level 100 mg/dL).

### Example 15

### Effects of Antisense Inhibition of PTP1B (ISIS 113715) on mRNA Expression in Liver

Male db/db mice and lean littermates (age 9 weeks at time 0) were divided into matched groups (n=6) with the same average blood glucose levels and treated by intraperitoneal injection once a week with saline, ISIS 29848 (the control oligonucleotide) or ISIS 113715. db/db mice were treated at a dose of 10, 25 or 50 mg/kg of ISIS 113715 or 50 mg/kg of ISIS 29848 while lean littermates were treated at a dose of 50 or 100 mg/kg of ISIS 113715 or 100 mg/kg of ISIS 29848. Treatment was continued for 4 weeks after which the mice were sacrificed and tissues collected for mRNA analysis. RNA values were normalized and are expressed as a percentage of saline treated control.

ISIS 113715 successfully reduced PTP1B mRNA levels in the livers of db/db mice at all doses examined (60% reduction of PTP1B mRNA), whereas the control oligonucleotide treated animals showed no reduction in PTP1B mRNA, remaining at the level of the saline treated control. Treatment of lean littermates with ISIS 113715 also reduced mRNA levels to 45% of control at the 50 mg/kg dose and 25% of control at the 100 mg/kg dose. The control oligonucleotide (ISIS 29848) failed to show any reduction in mRNA levels.

### Example 16

### Effects of Antisense Inhibition of PTP1B (ISIS 113715) on Body Weight

Male db/db mice and lean littermates (age 9 weeks at time 0) were divided into matched groups (n=6) with the same average blood glucose levels and treated by intraperitoneal injection once a week with saline, ISIS 29848 (the control oligonucleotide) or ISIS 113715. db/db mice were treated at a dose of 10, 25 or 50 mg/kg of ISIS 113715 or 50 mg/kg of ISIS 29848, while lean littermates were treated at a dose of 50 or 100 mg/kg of ISIS 113715 or 100 mg/kg of ISIS 29848. Treatment was continued for 4 weeks. At day 28 mice were sacrificed and final body weights were measured.

Treatment of ob/ob mice with ISIS 113715 resulted in an increase in body weight which was constant over the dose range with animals gaining an average of 11.0 grams while saline treated controls gained 5.5 grams. Animals treated with the control oligonucleotide gained an average of 7.8 grams of body weight.

Lean littermate animals treated with 50 or 100 mg/kg of ISIS 113715 gained 3.8 grams of body weight compared to a gain of 3.0 grams for the saline controls.

In a similar experiment, ob/ob mice and their lean littermates were dosed twice a week at 50 mg/kg with ISIS 113715, ISIS 29848 or saline control and body weights were measured at the end of day 7, 14 and 21.

Treatment of the ob/ob mice with ISIS 113715, ISIS 29848 or saline control all resulted in a similar increase in body weight across the 21-day timecourse. All of the lean littermate treatment groups showed a lesser increase in body weight which was equivalent among treatment groups.

Studies in ob/ob mice demonstrating that PTP1B antisense treatment can modulate fat storage and lipogenesis in adipose tissue have been published (Rondinone, C. M., Diabetes, 2002, 51(8), 2405-11).

These studies also show that PTP1B antisense treatment reduces expression of PTP1B protein in epididymal fat tissues as well as adiposity.

### Example 17

### Effects of Antisense Inhibition of PTP1B (ISIS 113715) on Plasma Insulin Levels

Male db/db mice (age 9 weeks at time 0) were divided into matched groups (n=6) with the same average blood glucose levels and treated by intraperitoneal injection twice a week with saline, ISIS 29848 (the control oligonucleotide) or ISIS 113715 at a dose of 50 mg/kg. Treatment was continued for 3 weeks with plasma insulin levels being measured on day 7, 14, and 21.

Mice treated with ISIS 113715 showed a decrease in plasma insulin levels from 15 ng/mL at day 7 to 7.5 ng/mL on day 21. Saline treated animals have plasma insulin levels of 37 ng/mL at day 7 which dropped to 25 ng/mL on day 14 but rose again to 33 ng/mL by day 21. Mice treated with the control oligonucleotide also showed a decrease in plasma insulin levels across the timecourse of the study from 25 ng/mL at day 7 to 10 ng/mL on day 21. However, ISIS 113715 was the most effective at reducing plasma insulin over time. This compound also decreases plasma insulin levels in ob/ob mice (Zinker et al., 2002, Proc. Natl. Acad. Sci. USA., 99, 11357-11362).

### Example 18

### Antisense Inhibition of PTP1B Expression (ISIS 113715) in Liver, Muscle and Adipose Tissue of the Cynomolgus Monkey

In a further embodiment, male cynomolgus monkeys were treated with ISIS 113715 (SEQ ID NO: 17) and levels of PTP1B mRNA and protein were measured in muscle, adipose and liver tissue. Serum samples were also measured for insulin levels.

Male cynomolgus monkeys were divided into two treatment groups, control animals (n=4; saline treatment only) and treated animals (n=8; treated with ISIS 113715). All animals had two pre-dosing glucose tolerance tests (GTTs) performed to establish insulin and glucose baseline values. Animals in the treatment group were dosed subcutaneously on days 1, 8, and 15 with 3 mg/kg, 6 mg/kg and 12 mg/kg of ISIS 113715, respectively. Animals in the control group were untreated. All animals had GTTs performed on days 5, 13 and 19, four days post-dosing. Ten days after the last dose of 12 mg/kg, all animals in the treatment group (ISIS 113715) received a one-time dose of 6 mg/kg of ISIS 113715. Three days later, all animals were sacrificed and tissues were taken for analysis of PTP1B mRNA and protein levels. Levels of mRNA and protein were normalized to those of the saline treated animals.

Of the tissue examined, PTP1B mRNA levels were reduced to the greatest extent in the fat and liver, being reduced by 41% and 40%, respectively. mRNA levels in muscle were reduced by 10%. Protein levels were reduced by 60% in the liver and by 45% in the muscle.

Levels of the liver enzymes ALT and AST were measured weekly and showed no change, indicating no ongoing toxic effects of the oligonucleotide treatment. Liver function tests were unremarkable after all doses and there were no reported changes in serum lipids. Over the course of the study there were no significant clinical signs other than one monkey that had slight swelling near the site of the 6 mg/kg SC injection. The subsequent 12 mg/kg injection in this monkey at a different injection site produced no observed changes. There was no evidence of toxicity associated with the rising dose regimen.

Fasting insulin and glucose values: Treatment of non-obese cynomolgus monkeys with ISIS 113715 reduced fasting plasma insulin levels. Fasting insulin concentrations were not decreased in control animals.

At the 5-week time point, plasma insulin levels in the ISIS 113715-treated animals were approximately 50% lower than baseline values (18.6+7.4 vs baseline 33.9+6.6 μU/ml*min, p<0.05). Increasing doses appeared to yield increasing effects. The decrease in fasting insulin levels was not associated with a change in fasted glucose concentration in either the treated or control group. In the control group, glucose levels varied from 48.0-51.5 mg/dL and in the treated groups the average values ranged from 53.0-54.0 mg/dL throughout the study. Alterations in glucose were not expected because these are normal animals, and hypoglycemia (plasma glucose <40 mg/dL) was not observed in any animal at any time point.

IVGTT data-glucose: Responses to a glucose challenge showed significant variability from animal to animal and day to day. There were no trends apparent when comparing the slopes of the glucose disappearance curve, an index of glucose utilization. There were no effects of ISIS 113715 on glucose AUC or maximum glucose concentrations observed during GTTs.

IVGTT data-insulin: Dose-dependent reductions in the AUC for insulin were observed in the treated animals and the area under the curve for the entire 60 minute period was reduced approximately 25% in ISIS 113715-treated animals compared to their baseline values at the highest dose (week 5: 9638±6431 vs baseline 12448±8047 μU/ml*min).

An index of insulin sensitivity can be derived from the ratio of the slope of the glucose disappearance curve (from 5 to 20 minutes) and the AUC of insulin. At week 5, there was a slight increase in insulin sensitivity in the ISIS 113715-treated group compared to baseline values (2.12±0.47 vs baseline 1.61±0.89, p=0.04). In control monkeys, this index of insulin sensitivity was unchanged at week 5 compared to baseline values (1.60±0.42 vs baseline 1.63±0.57).

### Example 19

### Effects of Antisense Inhibition of PTP1B (ISIS 113715) on mRNA Expression in Fractionated Liver

Male db/db mice (age 9 weeks at time 0) were divided into matched groups (n=6) with the same average blood glucose levels and treated by intraperitoneal injection once a week with saline, ISIS 29848 (the control oligonucleotide) or ISIS 113715. db/db mice were treated at a dose of 50 mg/kg of ISIS 113715 or 50 mg/kg of ISIS 29848 or 100 mg/kg of ISIS 29848. Treatment was continued for 3 weeks after which the mice were sacrificed and tissues were collected for analysis. Liver tissue was removed and homogenized whole or fractionated into hepatocytes and non-parenchymal (NP) cell fractions by standard methods (Graham et al., J. Pharmacol. Exp. Ther., 1998, 286, 447-458). During the study, plasma glucose levels were measured as were PTP1B mRNA levels in both cell fractions. RNA values were normalized and are expressed as a percentage of saline treated control.

Treatment of db/db mice with ISIS 113715 caused a significant reduction in plasma glucose levels (saline=500+/−25 vs. treated=223+/−21 mg/dL; p=0.0001).

ISIS 113715 successfully reduced PTP1B mRNA levels in both hepatocytes and NP cell fractions, with an 80% reduction in hepatocytes and a 30% reduction in the NP cell fraction. In addition, PTP1B expression in the two cell fractions was found to be dramatically different with a 5-8 fold greater level of expression being found in the NP fraction.

### Example 20

### Effects of Antisense Inhibition of PTP1B Expression (ISIS 113715) in the Obese Insulin-Resistant Hyperinsulinemic Rhesus Monkey-Improved Insulin Sensitivity

In a further embodiment, five male obese insulin-resistant hyperinsulinemic Rhesus monkeys were treated with ISIS 113715 (SEQ ID NO: 17) and insulin sensitivity, glucose tolerance and PTP1B mRNA and protein were measured. Serum samples were also measured for insulin levels. These animals, though obese, were normoglycemic and therefore the primary endpoints were a reduction in fasted insulin and GTT insulin levels.

All animals had two pre-dosing glucose tolerance tests (GTTs) performed to establish insulin and glucose baseline values. Animals were dosed subcutaneously in the interscapular region at a dose of 20 mg/kg (3 injections on alternate days the first week followed by one injection per week for the next three weeks). Fasted glucose/insulin levels and glucose tolerance (IVGTTs) were measured as pharmacologic endpoints. Fasting samples were collected during the second week, 48 hr after dosing. An IVGTT was performed during the third week, 48 hours post-dosing.

As compared to baseline values, a 50% reduction in fasting insulin levels was observed (treated: 31±9 vs. baseline: 67±7 μU/mL, p=0.0001), which was not accompanied by any change in plasma glucose levels. Furthermore, a marked reduction in insulin levels (AUC) was observed after IVGTTs (treated: 7295±3178 vs. baseline: 18968±2113 μU-min/mL, p=0.0002). Insulin sensitivity was also significantly increased (glucose slope/insulin AUC; 5-20 minutes).

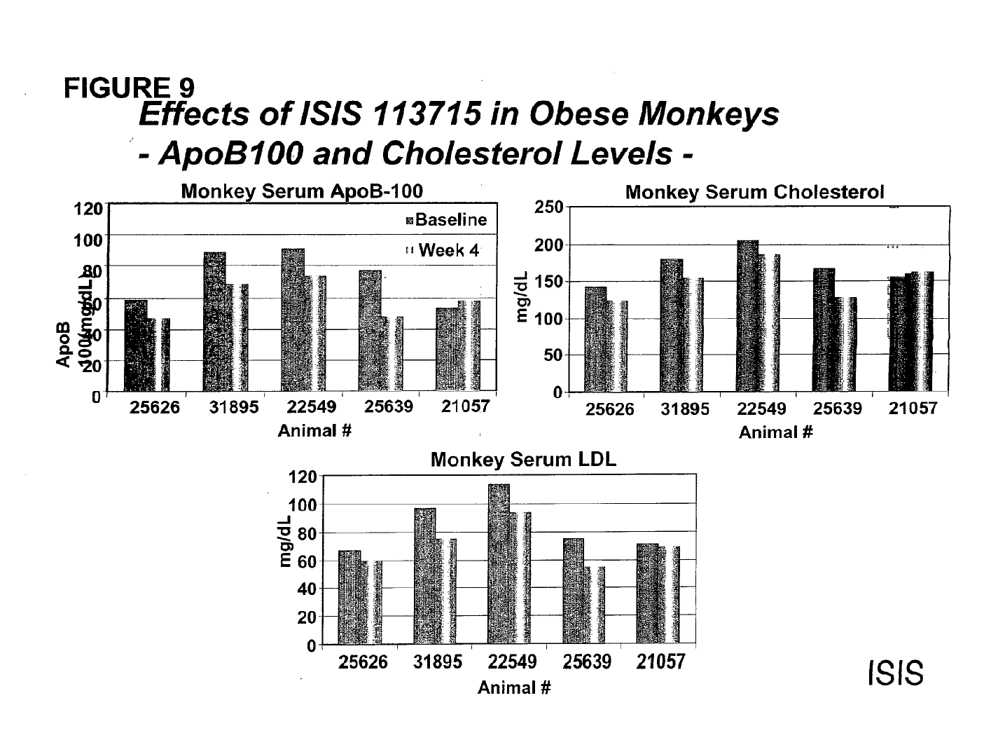

Hypoglycemia was not observed, even in the 16 hour-fasted animals. Levels of the liver enzymes ALT and AST were measured weekly and showed no change, indicating no ongoing toxic effects of the oligonucleotide treatment. Renal function tests were also normal. There were no significant clinical signs including any subcutaneous reactions at the injection site. There was a trend toward a reduction in serum triglycerides (from 131 mg/dL at baseline to 93 mg/dL after treatment). In addition, apolipoprotein B (apoB) levels were reduced as compared to baseline in the majority of the animals treated (for n=5 per group, the average was about 74 mg/dL at baseline, and 59 mg/dL post-treatment). As shown in FIG. 9, serum cholesterol (for n=5 per group, the average was about 174 mg/dL at baseline and 161 mg/dL post-treatment) and serum LDL levels were reduced (for n=5 per group, the average was about 84 mg/dL at baseline and 70 mg/dL post-treatment).

Adiponectin is believed to be positively correlated with insulin sensitivity, particularly in peripheral tissues, i.e. skeletal muscle. Low plasma adiponectin concentrations have been found to precede a decline in insulin sensitivity. Stefan et al., 2002, Diabetes 51, 1884-1888. Adiponectin levels in plasma were measured at baseline and week 4 of ISIS 113715 treatment of the obese rhesus monkeys using a commercially available human adiponectin ELISA assay kit. Plasma adiponectin levels were found to double during the four weeks of treatment with ISIS 113715.

The results of this study are consistent with those seen in previous rodent and monkey studies described herein which demonstrate a significant lowering of insulin levels suggesting that insulin efficiency (sensitivity) was increased upon treatment with ISIS 113715. These findings are consistent with all previous findings in rodent studies. It should be noted that these animals were normoglycemic and the endpoint effect on insulin levels is believed to be less sensitive than lowering glucose levels in frankly diabetic animals.

### Example 21

### Effects of ISIS 113715 in High-Fat Fed Mice

Four-week old male C57BL/6J mice (Jackson Laboratories) were placed on a high-fat (60% fat) diet for four weeks. Subsequently, the mice were treated with either saline, mismatch control oligonucleotide (ISIS 141923; CCTTCCCTGAAGGTTCCTCC; SEQ ID NO: 20) or ISIS 113715 (SEQ ID NO: 17) at a dose of 50 mg/kg once a week by intraperitoneal injection for 6 weeks (n=10/treatment group). The animals were weighed once a week and a GTT was performed 4 weeks after treatment initiation. At the end of 6 weeks, the mice were sacrificed and the liver was removed. Tissue extracts were prepared and were subjected to Western blot analysis to measure changes in PTP1B protein levels.

Significantly reduced weight gain was seen in high-fat fed mice treated with ISIS 113715. At the end of six weeks, ISIS 113715-treated mice had gained 45% less body weight compared to saline or control oligonucleotide-treated mice and had a similar reduction in epididymal fat pad weights. Serum insulin concentration in the ISIS 113715-treated mice was reduced to that seen in normal chow-fed mice (high fat-fed: 2.8±0.3; ISIS 113715-treated: 0.9±0.3; normal chow: 1.1±0.5 ng/ml) and the mice also performed better on a glucose tolerance test (maximum blood glucose excursion went from approximately 125 mg/dL at time 0 to approximately 300 mg/dL at 30 min.; compared to saline-treated animals on the high fat diet which had glucose excursion from approx 175 mg/dL at time 0 to approximately 430 mg/dL at 30 min and a maximum of approximately 460 mg/dL at 60 min. Mice on normal diet had glucose excursion of from approximately 100 mg/dL at time 0 to a maximum of approximately 175 mg/dL at 30 minutes). PTP1B protein expression was reduced by approximately 50% in livers of animals treated with ISIS 113715. Thus PTP1B antisense treatment increased insulin sensitivity and reduced weight gain in normal mice fed a high fat diet.

### Example 22

### Effect of Antisense Inhibitors of PTP1B Receptor on Zucker Diabetic Fatty (ZDF) Rats

The leptin receptor deficient Zucker diabetic fatty (ZDF) rat is another useful model for the investigation of type 2 diabetes. Diabetes develops spontaneously in these male rats at ages 8-10 weeks, and is associated with hyperphagia, polyuria, polydipsia, and impaired weight gain, symptoms which parallel the clinical symptoms of diabetes (Phillips M S, et al., 1996, Nat Genet. 13, 18-19).

ZDF/GmiCrl-fa/fa (ZDF) male rats were purchased from Charles River Laboratories (Wilmington, Mass., USA). Six week old ZDF male rats were injected intraperitoneally with oligonucleotides at a dose of 25 mg/kg two times per week for four weeks. PTP1B antisense oligonucleotides used were ISIS 113715 (SEQ ID NO: 17) and ISIS 106425 (TGAACAGGTTAAGGCCCTGA; SEQ ID NO: 21), a 2′-MOE gapmer with phosphorothioate backbone which is complementary to mouse and rat PTP1B. ISIS 141923 (SEQ ID NO: 20), a six-mismatch control of ISIS 113715, was used as the negative oligonucleotide control. Saline-injected animals also serve as controls.

In ZDF rats treated with ISIS 113715 (SEQ ID NO: 17), an antisense inhibitor of PTP1B, fed plasma glucose levels were approximately 274±43 mg/dL at week 0, 302±51 mg/dL at week 1, 315±44 mg/dL at week 2, 320±43 mg/dL at week 3 and 299±46 mg/dL at week 4. In rats treated with ISIS 106425 (SEQ ID NO: 415), another antisense inhibitor of PTP1B, fed plasma glucose levels were approximately 275±43 mg/dL at week 0, 302±53 mg/dL at week 1, 293±50 mg/dL at week 2, 315±54 mg/dL at week 3 and 272±41 mg/dL at week 4. In contrast, rats treated with saline alone had fed plasma glucose levels of approximately 302±44 mg/dL at week 0, 400±17 mg/dL at week 1, 441±13 mg/dL at week 2, 453±26 mg/dL at week 3 and 425±10 mg/dL at week 4. Rats treated with negative control oligonucleotide ISIS 141923 had fed plasma glucose levels of approximately 306±59 mg/dL at week 0, 391±35 mg/dL at week 1, 402±37 mg/dL at week 2, 411±27 mg/dL at week 3 and 392±11 mg/dL at week 4.

An intraperitoneal glucose tolerance test (IPGTT) was also performed. Rats received intraperitoneal injections of glucose, and the blood glucose and insulin levels are measured before the glucose challenge and at intervals over 2 hours. The blood glucose levels (in mg/dL) are shown below in Table 1:

TABLE 1
Blood glucose levels (mg/dL) in ZDF rats after IPGTT following
treatment with antisense inhibitor of PTP1B

| | Mins: | | | | | |
|---|---|---|---|---|---|---|
| | 0 | 5 | 15 | 30 | 60 | 120 |
| Compounds | Glucose mg/dL ± SEM | | | | | |
| saline | 302 ± 34 | 466 ± 59 | 563 ± 83 | 625 ± 77 | 609 ± 61 | 558 ± 51 |
| Control ISIS141923 | 330 ± 39 | 543 ± 43 | 703 ± 60 | 791 ± 52 | 705 ± 51 | 594 ± 28 |
| PTP1B ISIS113715 | 206 ± 37 | 477 ± 46 | 574 ± 39 | 586 ± 51 | 553 ± 51 | 426 ± 58 |
| PTP1B | 195 ± 42 | 557 ± 49 | 648 ± 51 | 624 ± 44 | 546 ± 50 | 375 ± 61 |
| ISIS 106425 | | | | | | |

The blood glucose levels shown in Table 1 were graphed over time and the area under the curve (AUC) is calculated. A smaller AUC for glucose (smaller blood glucose excursion after glucose challenge) indicates better glucose tolerance. Glucose excursion (AUC) for saline-treated rats was approximately 72,000; for negative control ISIS 141923-treated rats, approximately 80,000, for ISIS 113715-treated rats, approximately 60,000 and for ISIS 106425-treated rats, approximately 63,000.

Insulin excursion after the IPGTT was also increased as shown in Table 2.

TABLE 2
Insulin excursion (ng/ml) after intraperitoneal glucose
tolerance test (IPGTT) in ZDF rats

| | Time (min): | | | | | |
|---|---|---|---|---|---|---|
| | 0 | 5 | 15 | 30 | 60 | 120 |
| Compound | Insulin (ng/ml ± SEM) | | | | | |
| Saline | 2.2 ± .37 | 2.4 ± .32 | 3.8 ± .11 | 4.1 ± .52 | 2.3 ± .79 | 1.0 ± .26 |
| Control | 2.2 ± .30 | 2.3 ± .84 | 4.0 ± .22 | 4.2 ± .37 | 2.4 ± .66 | 1.7 ± .60 |
| 141923 | | | | | | |
| PTP1B | 1.9 ± .57 | 3.8 ± 1.26 | 4.7 ± .98 | 4.7 ± .82 | 2.9 ± 1.52 | 2.9 ± 1.94 |
| 113715 | | | | | | |
| PTP1B | 3.8 ± 1.58 | 4.9 ± 1.57 | 6.7 ± 1.36 | 6.1 ± 1.5 | 6.6 ± 2.07 | 4.2 ± 2.23 |
| 106425 | | | | | | |

The values in Table 2 were graphed over time to give AUC values. AUCs were approximately 300 for saline-treated rats, 320 for ISIS 141923 control-treated rats, approximately 400 for ISIS 113715-treated animals and approximately 680 for ISIS 106425-treated animals.

Plasma transaminases (AST and ALT) were not significantly altered by treatment with either PTP1B antisense oligonucleotide compared to saline treated rats, indicating a lack of liver toxicity.

PTP1B protein levels were measured by Western blot analysis. Compared to saline-treated animals, PTP1B levels were decreased by 10% after treatment with ISIS 141923, by about 55% after treatment with ISIS 113715 and by about 50% after treatment with ISIS 106425.

### Example 23

### Effect of Antisense Inhibitors of PTP1B Receptor on Zucker Diabetic Fatty (ZDF) Rats—Comparison to Rosiglitazone and Metformin

Seven-week old male ZDF rats were treated with either saline or ISIS 113715 at a dose of 50 mg/kg/week by intraperitoneal (IP) injection for 5 weeks (n=7 for saline and n=10 for ISIS 113715). The effects of ISIS 113715 were compared with rosiglitazone (10 mg/kg/day orally, n=8) or metformin (800 mg/kg/day orally, n=8). A saline treated group of lean rats was also included in the study. Treatment with all drugs was initiated before the rats became frankly diabetic, which occurs at about 8 weeks of age. Plasma glucose levels were measured 3 and 5 weeks after treatment initiation. Body weight was measured once every week; in addition, HbA, levels were measured at the end of the study by HPLC. A meal tolerance test was also performed after 4 weeks of treatment.

At the end of 5 weeks, some rats were sacrificed and the liver and epididymal fat pads were removed. The rest of the animals (n=4/group) were examined for changes in plasma glucose levels after cessation of treatment. Glucose measurements during the recovery phase were made 10 days and 5 weeks after stopping treatment. Results are shown in Table 3. Tissue extracts were subjected to Western blot analysis to measure changes in PTP1B protein levels.

TABLE 3
Blood glucose after treatment of ZDF rats with ISIS 113715
compared to rosiglitazone or metformin

| | Time | | | |
|---|---|---|---|---|
| | | Week 3 of | | 31 day |
| | Pre-treatment | dosing | 10 day washout | washout |
| Compound | Blood glucose (mg/dL) (approx.) | | | |
| Saline | 140 | 320 | 310 | 320 |
| ISIS 113715 | 135 | 175 | 210 | 180 |
| Rosiglitazone | 140 | 110 | 260 | 270 |
| Metformin | 135 | 180 | 305 | 330 |
| Lean rats | 95 | 100 | 100 | 80 |

ISIS 113715 prevented or delayed the progression of diabetes in ZDF rats. The glucose lowering effects of ISIS 113715 were sustained for up to 5 weeks following cessation of treatment; such durable control was not seen with either rosiglitazone or metformin treatment.

HbA_{1c} was reduced in all drug treatment groups compared to saline (9.2% for saline-treated ZDF rats, 5.5 for saline-treated lean rats, 6.8 for ISIS 113715-treated ZDF rats, 5.4 for rosiglitazone-treated ZDF rats and 6.1 for metformin-treated ZDF rats. All treatments yielded statistically significant (p<0.001) decreases in % HbA_{1c} compared to saline-treated animals. There is a strong correlation between levels of HbA_{1c} and the average blood glucose levels over the previous 3 months (for humans; one month for rodents due to faster turnover of red blood cells), and thus HbA_{1c} is a measure of sustained blood glucose control (Bunn, H. F. et al., 1978, Science. 200, 21-7).

Meal tolerance tests indicated that ISIS 113715 caused an improvement in glucose excursion, indicating improved glucose tolerance. Similar effects were observed with rosiglitazone and metformin at the high doses used in this study. Body weight remained unchanged after treatment with ISIS 113715 or metformin, but was increased after rosiglitazone treatment.

### Example 24

### Preclinical Toxicology and Pharmacokinetics—Summary

Safety studies completed with ISIS 113715 include a 2-week rat toxicity study, a 3 month rat toxicity study and two 3-month monkey toxicity studies. ISIS 113715 targets human, mouse, rat and monkey PTP1B with perfect homology.

Tissue concentrations of ISIS 113715 following 13 weeks of treatment were generally higher in monkeys than observed in rats at comparable dose levels (see Table 4 below). The observed distribution and accumulation in tissues of pharmacological interest is generally favorable for clinical application of ISIS 113715.

TABLE 4
Parent drug (ISIS 113715) concentrations (μg/g) in selected organs or tissues

| | Tissue | | | | | |
|---|---|---|---|---|---|---|
| | Rat | | Monkey | | | |
| | Dose mg/kg | | | | | |
| | 3 | 30 | 1 | 3 | 10 | 20 |
| | Route | | | | | |
| | IV bolus | IV bolus | IV infusion | IV infusion | IV infusion | SC |
| Liver | 37.5 ± 9.6 | 692 ± 59.2 | 59.0 ± 25.6 | 110 ± 6.0 | 301 ± 88.1 | 678 ± 95.2 |
| kidney^{a} | 240 ± 54.6 | 844 ± 246 | 200 ± 34.8 | 472 ± 111 | 1369 ± 619 | 2651 ± 1080 |
| Fat | 1.0 ± 1.1 | 10.0 ± 3.9 | 0.5 ± 0.8 | 9.9 ± 4.7 | 37.3 ± 14.4 | 67.0 ± 33.0 |
| Skeletal | 2.3 ± 0.6 | 13.9 ± 3.3 | <0.35 | <0.35 | 2.0 ± 1.1 | 2.2 ± 0.5 |
| muscle | | | | | | |

^{a}whole kidney in rat; kidney cortex in monkey

In addition, tissue concentrations after subcutaneous administration were comparable to those following IV administration in monkeys (when adjusted for dose), indicating complete systemic absorption by this route. The tissue distribution and elimination of ISIS 113715 following SC injection were similar to those produced with IV infusion suggesting that systemically absorbed ISIS 113715 is distributed independently of the route of administration. However the apparent plasma half-life was longer following SC injection (200 to 300 min) due to the ongoing slow absorption process.

The clearance of ISIS 113715 from tissues was very slow relative to plasma clearance. The tissue half-lives in rats were 8.6 to 35 days (measured radiolabel) and 8 days to 23 days in monkey (measured parent drug). This slow clearance supports infrequent dosing. Following the loading phase (dosing every third day for three doses), tissue concentrations were approximately 2-3-fold higher in rats and monkeys compared to single dose concentrations. Maintenance dosing (once weekly for 3 months) either maintained concentrations or produced an additional 1- to 2-fold increase in concentration.

### Example 25

### Frequently Sampled Intravenous Glucose Tolerance Test (IVGTT) Protocol

Human subjects/patients are fasted for 12 hours prior to testing. On day of test, subjects are weighed and the volume of 50% glucose solution to be infused is calculated by the following formula:

Subject weight in kg×0.6=# ml of glucose solution to be infused.

A pre-test blood sample is drawn, centrifuged and 2 mL of subject serum is reserved. 30 ml of 1 U/ml solution of insulin is prepared using regular human insulin (0.3 ml of 100 U/ml Humalin, Novolin-R or equivalent) and saline (27.7 ml) and subject's serum (or Human Serum Albumin), (2 ml). The required insulin dose is calculated from the following formulae:

Insulin dose=body weight (kg)×0.03 U/kg=______ units of insulin.

Insulin volume=1 U/ml solution×______ units=______ ml of diluted insulin solution (round to nearest 0.5).

A cannula is placed in each antecubital vein or hand of the patient. One cannula is connected to a bag of normal saline which is infused at a rate of ˜0.5 ml/min to maintain cannula patency. This cannula is used to inject the glucose and insulin solutions.

In the other arm, a second cannula is connected to a second bag of normal saline which is infused at a rate of 0.5 ml/min. This cannula is used to draw all the IVGTT blood samples.

Blood samples are drawn at −20, −10 and 1 minutes before the 50% glucose bolus injection. Immediately following the 0 minute blood draw, the 50% glucose infusion is administered into the opposite arm as a smooth bolus over one minute. Additional blood samples are drawn at exactly 2, 3, 4, 5, 6, 8, 10, 14 and 19 minutes after glucose injection. All IVGTT samples are placed on ice and centrifuged and frozen within one hour of being drawn.

At exactly 20 minutes after completing the glucose bolus injection, the insulin injection is administered in the same arm that received the glucose injection. Additional blood samples are drawn at 22, 24, 27, 30, 40, 50, 70, 90, 120, 150, 180 and (optionally, depending on protocol) 240 minutes. All IVGTT samples are placed on ice and centrifuged and frozen within one hour of being drawn.

### Example 26

### Human Clinical Trials of ISIS 113715—A Double-Blind, Placebo-Controlled, Dose-Escalation, Phase 1/2A Study to Assess the Safety, Tolerability, Pharmacokinetics and Pharmacodynamics of Single and Multiple Doses of ISIS 113715 Administered Intravenously to Healthy Volunteers and Type 2 Diabetics (CS-1)

A Phase I clinical trial (CS1) was conducted to assess the safety and pharmacokinetic profile of increasing doses of ISIS 113715 given intravenously to 20 healthy volunteers.

Inclusion criteria for the study population were as follows:

1. Age: 18 to 65 years;

2. Gender: male or female although females must be post-menopausal or surgically sterile.

3. Give written informed consent to participate in the trial.

Exclusion Criteria:

1. Pregnant women or nursing mothers;

2. Clinically significant abnormalities in medical history or physical examination

3. Clinically significant abnormalities on laboratory examination;

4. Clinically significant abnormalities in complement;

5. Clinically significant abnormalities in coagulation parameters;

6. Subjects with serum creatinine levels greater than or equal to 1.2 mg/dL for females or greater than or equal to 1.5 mg/dL for males, or creatinine clearance outside the normal range for the subject's age and gender.

7. HIV positive;

8. Active infection requiring antiviral or antimicrobial therapy;

9. Receiving prescription medication with the exception of estrogen replacement therapy;

10. Malignancy (with the exception of basal or squamous cell carcinoma of the skin if adequately treated and no recurrence for >1 year;

11. Uncontrolled chronic disease;

12. Any other concurrent condition which, in the opinion of the investigator, would preclude participation in the study or interfere with compliance;

13. History of current alcohol or drug abuse;

14. Subjects smoking more than 10 cigarettes per day;

15. Subject weighing more than 10% above or below ideal body weight;

16. Undergoing or have undergone treatment with another investigational drug, biologic agent or device within 90 days prior to screening;

17. Blood donation within 3 months of screening.

This trial had five initial cohorts (A-E), four subjects per cohort. Four subjects were randomized in a 3:1 ratio to receive ISIS 113715 or placebo, respectively, within each cohort. Each cohort received a different dosage of ISIS 113715. Cohort A-0.5 mg/kg ISIS 113715 or placebo; Cohort B-1.0 mg/kg ISIS 113715 or placebo; Cohort C-2.5 mg/kg ISIS 113715 or placebo; Cohort D-5.0 mg/kg ISIS 113715 or placebo; Cohort E-7.5 mg/kg ISIS 113715 or placebo). Cohort B single dosing began after Cohort A single dosing was complete. Multi dosing began after safety review of data from single dosing of cohorts A and B. For multidosing, a single dose was given, then after a 2-4 week period of rest, three additional doses were given over 5 days. ISIS 113715 was administered as a two-hour continuous intravenous infusion. ISIS 113715 was provided as a 10 mg/ml solution in sterile, unpreserved, buffered saline which is diluted if necessary.

Blood and urine samples were collected for chemistry, CBC, coagulation, complement, urinalysis, and ISIS 113715 concentration at selected timepoints following study drug administration. Blood sampling for ISIS 113715 PK analysis were also collected on days 1, 2 and 4 of treatment. A fasting 120-minute intravenous glucose tolerance test (IVGTT) was done before dosing and five days post-dosing for cohorts D and E only (5.0 and 7.5 mg/kg cohorts). The more extensive fasting 240-minute frequently sampled IVGTT may also be performed, as described in other examples herein.

The results are summarized as follows: ISIS 113715 was administered to 15 healthy volunteer patients at single and multiple doses of 0.5, 1.0, 2.5, 5.0 and 7.5 mg/kg body weight in this Phase 1 study. During the single dose component of the study, patients received a single dose of ISIS 113715 or placebo at the above doses. This was followed 3 to 4 weeks later with the multiple dose component of the study in which ISIS 113715 or placebo was administered thrice over a 5-day period.

There were no clinically relevant changes in laboratory parameters including EKG, urinalysis and chemistry, hematology and coagulation panels. There was a transient, dose-related prolongation of the aPTT (40±2 seconds in the 5.0 mg/kg dose cohort and 51±9 seconds in the 7.5 mg/kg cohort, compared to a normal reference range of 26-43 seconds) which was without clinical sequelae. Analysis of the Complement split products, C5a and Bb, revealed no changes from baseline.

Plasma concentrations of ISIS 113715 were determined following single and multiple doses. Maximal concentrations (Cₘₐₓ) were seen at or near the end of the 2-hour infusion followed by a multi-phasic decline with an initial, relatively fast distribution phase (0.5 to 1.9 hours mean half-life) that dominated the plasma clearance, followed by at least one slower disposition phase. Following both single and multiple dosing, Cₘₐₓ exhibited a dose-proportional increase, while AUCₜₗₐₛₜ had a greater than dose-proportional increase, which corresponded to a decrease in plasma clearance at the higher doses. The dose-dependent decrease in plasma clearance is likely due, in part, to saturation of tissue distribution at higher doses. This has also been observed in preclinical models. The clearance was essentially linear (dose-independent) over the doses of 2.5 to 7.5 mg/kg. Both the Cₘₐₓ and the AUCₜₗₐₛₜ of ISIS 113715 were similar between single and multiple dosing regardless of the dose, suggesting no accumulation of the drug in plasma over the dosing period.

No serious adverse events (SAEs) were reported during the study. 72 adverse events (AEs) were reported; i.e., by 5 placebo-treated patients and 14 patients who received ISIS 113715. The ratio of AE in patients who received active drug versus placebo-treated patients was 3.2:1; this ratio is similar to the 3:1 study randomization. The number of AE was evenly distributed among dose cohorts with the exception of the 0.5 mg/kg cohort in which only 5 AE were reported. The majority (46) of the AE were deemed by the Investigator to be unrelated to study drug while the remaining 26 were considered possibly related. The AE were nonspecific and ranged from headache, insomnia and somnolence to hematoma at the site of cannulation.

The pharmacologic activity of ISIS 113715 was examined in the 5.0 and 7.5 mg/kg dose cohorts with an intravenous glucose tolerance test. The area under the curve (AUC) for insulin, glucose and C-peptide was determined; this preliminary analysis is shown in Table 5.

TABLE 5
AUC preliminary analysis
ISIS 113715 (n = 6) vs Placebo (n = 2)

| Mean % change | 5.0 mg/kg | 7.5 mg/kg | Placebo |
|---|---|---|---|
| Insulin AUC | −27 | −32 | 19 |
| Glucose AUC | 0.3 | 1.3 | 6.0 |
| C-peptide AUC | −13 | −4.4 | 11 |

As might be expected for nondiabetic patients, there were no changes in the glucose excursion and AUCs prior to and after ISIS 113715 administration in either cohort. There was no indication of hypoglycemia. However, insulin AUCs decreased by 27% and 32% in the 5.0 and 7.5 mg/kg cohorts, respectively, relative to baseline. In contrast, insulin AUCs increased by 19% after ISIS 113715 administration relative to baseline in the 2 placebo-treated patients.

This study demonstrated improved glucose tolerance and improved insulin sensitivity (as measured by glucose tolerance test) in all subjects who received ISIS 113715, and the drug was well tolerated. No hypoglycemia was observed. These results are consistent with the preclinical pharmacology in rodents and non-human primates.

Based on these encouraging results in normal patient volunteers, ten patients with Type 2 diabetes were added to this trial as Cohort F. Inclusion criteria were as for Cohorts A-E plus the following:

1. Patients have Type 2 diabetes of less than 8 years duration since diagnosis;

2. Patients are on stable dose of oral sulfonylurea (glibenclamide, glipizide or glimepride) for at least 3 months prior to screening;

3. Patients have fasting blood glucose between 125 and 200 mg/dl;

4. Patients have HbA, of 7-10%;

5. Patients have body mass index less than or equal to 32 kg m⁻².

Cohort F patients were randomized in a 7:3 ratio to receive ISIS 113715 (5.0 mg/kg, not to exceed 400 mg per dose) or placebo, respectively. Following the multiple dose drug treatment period, subjects in Cohort F entered a 15-day extension period and received 3 additional doses of ISIS 113715 (one infusion per week). Patients are evaluated as for Cohorts A-E above.

### Example 27

### Human Clinical Trials—Phase II—A Double-Blind, Placebo-Controlled, Dose-Escalation Study to Assess the Safety, Tolerability, Pharmacokinetics and Activity of ISIS 113715 in Patients with Type 2 Diabetes who have not Received Prior Therapy (CS-7)

Phase II clinical trials are underway to further evaluate the ability of ISIS 113715 to regulate blood glucose levels in patients with type 2 diabetes. Type 2 diabetics enrolled in the study (5 cohorts) are dosed intravenously with 100, 200, 400 or 600 mg of ISIS 113715.

Cohort A: 100 mg ISIS 113715 or placebo; Cohort B: 200 mg ISIS 113715 or placebo; Cohort C: 400 mg ISIS 113715 or placebo; Cohort D: 600 mg ISIS 113715 or placebo, Cohort E: 200 mg ISIS 113715 or placebo. Approximately sixteen patients will be randomized in a 3:1 ratio (ISIS 113715 to placebo) into each of four dose cohorts (Cohorts A-D), and approximately thirty-two patients will be randomized in a 3:1 ratio (ISIS 113715 to placebo) into Cohort E, for a total of 96 patients.

The dose range chosen for this study, 100-600 mg, is equivalent to 1.43 to 8.57 mg/kg for a 70-kg patient. Since patients with Type 2 diabetes are often obese, the actual exposure is likely less (1 to 6 mg/kg for a 100-kg patient). These doses are comparable to those (0.5 to 7.5 mg/kg) that were safely administered to healthy volunteer patients in the Phase I study (CS1). In that study, there were 27% and 32% decreases in insulin AUCs following 5.0 or 7.5 mg/kg dose cohorts following IVGTT challenges. While lower doses were not challenged with IVGTT in that study, preclinical experience with antisense oligonucleotides predicts that doses as low as 2 mg/kg will exhibit pharmacology.

Patients receive three loading doses in week 1, then are dosed once a week for weeks 2-6 for a total of six weeks. The alternate day administration (loading dose) for the initial week of the study was chosen so that steady state can be achieved rapidly. With tissue half-lives of 10-30 days, achieving steady state is not instantaneous. Data from pharmacokinetic studies show that the loading dose strategy results in organs reaching approximately 70% to 80% steady-state levels during the first week. This thrice-weekly schedule was used in the Phase I clinical study (CS1). Study drug will be administered as three loading doses via a 1-hour intravenous infusion for Cohorts A, B, C, and E, and a 2-hour infusion for Cohort D on Days 1, 3 and 5 of Week 1. Study drug will be administered once weekly via intravenous infusion during the remaining weeks of the treatment period.

The CS7 study for an individual patient consists of a 2-week screening period, 3-week baseline period, 6-week treatment period, and a post-treatment evaluation period. For patients in Cohort E, the treatment period was 12-weeks. For patients in Cohort A and most patients in Cohort B, the post-treatment evaluation period was 4-weeks. For some patients in Cohort B and all patients in Cohorts C-E, the post-treatment period was 12-weeks. For the study, inclusion criteria include: age 18 to 65 years, male or female gender although females must be post-menopausal or surgically sterile, Type 2 diabetes mellitus of less than 5 years in duration, have never received hypoglycemic therapy, fasting blood glucose between 130 and 220 mg/dL (7.2 to 12.2 mmol/L for Cohorts A-D and between 140 and 220 mg/dL (7.8 and 12.2 mmol/L) for Cohort E, HbA_{1c} between 6.8 and 10.0% for Cohorts A-D and between 7.5 and 11.0% for Cohort E, body mass index greater than 25 and less than 35 kg m⁻², and given written informed consent to participate in the study.

Exclusion criteria include: medication that may affect glucose homeostasis (e.g. systemic glucocorticoid) within one month of screening, clinically significant abnormalities in medical history or physical exam, clinically significant abnormalities on laboratory examination, history of HIV infection, active infection requiring antiviral or antimicrobial therapy, malignancy (with the exception of basal or squamous cell carcinoma of the skin if adequately treated and no recurrence for more than one year at the time of screening, any other condition which in the opinion of the investigator would preclude participation in or interfere with compliance, alcohol or drug abuse, undergoing or have undergone treatment with another investigational drug, biologic agent, or device within 90 days of screening, abnormal serum creatinine concentration defined as greater than 1.5 mg/dL for males and greater than 1.2 mg/dL for females, medications that may affect coagulation (heparin, warfarin, etc.) with the exception of acetylsalicylic acid or non-steroidal anti-inflammatory agents, and allergy to sulfur-containing medications.

An ECG is performed in Weeks 3 and 6 (Cohorts A-D and in Weeks 3, 6, 9, and 12 (Cohort E) prior to study drug infusion. An FSIVGTT following an overnight fast (at least 12 hours) is performed on Day 3 of Week 6 for Cohort A and the first group of eight patients in Cohort B. An abbreviated procedure (blood samples collected for glucose, insulin and C-peptide at three time points, and an additional sample for HbA₁₁) is performed prior to study drug infusion at Week 6 (Cohorts C, D and the second group of eight patients in Cohort B) and at Weeks 3, 6, and 12 (Cohort E).

Blood samples for ISIS 113715 PK analysis are collected at each visit during treatment for the first 12 patients of each cohort. In addition, urine is collected for 24 hours in Week 1 (Day 1), and in either Week 6 (Cohorts A-D) or Week 12 (Cohort E) for PK evaluation. The remaining patients within each cohort undergo an abbreviated PK evaluation with blood samples collected at Weeks 1 (Day 1), 2, 4, and 6 (Cohorts A-D) and Weeks 1 (day 1), 2, 4, 6, 8, 10, and 12 (Cohort E).

ISIS 113715 is provided as a 250 mg/ml solution in sterile, unpreserved buffered saline. Placebo is 0.9% saline with riboflavin for coloring. ISIS 113715 is administered as three loading doses via a 1-hour intravenous infusion for Cohorts A-C and a 2-hour infusion for Cohort D on days 1, 3 and 5 of week 1. Blood samples for coagulation and complement are collected 2.5 and 4 hours following the start of study drug infusion on day 1. Blood is collected for additional safety labs. Patients return to the study center on days 3 and 5 for drug infusion, reporting of adverse events, and lab tests for safety analysis. Patients will measure their fasted blood glucose level at home daily.

Blood samples for ISIS 113715 PK analysis will be collected at each visit during treatment for the first 12 patients of each cohort. In addition, urine will be collected for 24 hours in Week 1 (Day 1), and in either week 6 (cohorts A-D or week 12 (cohort E) for PK evaluation. The remaining patients within each cohort will undergo an abbreviated PK evaluation with blood samples collected at Weeks 1 (Day 1), 2, 4, and 6 (Chohorts A-D) and Weeks 1 (Day 1), 2, 4, 6, 8, 10, and 12 (Cohort E).

This study is intended as a safety and tolerability study and was not designed or powered to examine efficacy.

The activity of 11315 in this study is assessed by comparing the end of-ISIS-113715-treatment changes for HbA_{1c}, fasting blood glucose, insulin, and C-peptide to placebo. Additional analyses of derived measures of insulin sensitivity and β-cell function throughout the entire study will also be conducted. Measures of insulin sensitivity include the Quantitative Insulin Sensitivity Check Index (QUICKI). The QUICKI index is computed as 1/log 10 (fasting plasma glucose [mg/dL]×fasting insulin [μU/mL]). β-cell function will be estimated using the Homeostasis Model Assessment (HOMA) β-cell function index. This index is calculated as 20× fasting insulin (μU/mL)/((fasting plasma glucose (mg/dL)/18)-3.5). Lipid measures will include triglycerides, HDL, LDL, and VLDL cholesterol, and total cholesterol. Derived lipid measures include the ratio of total cholesterol/HDL cholesterol and the ratio of HDL to LDL cholesterol. The effects of ISIS 113715 on measures of glycemic control (HbA_{1c}, fasting plasma glucose, daily blood glucose measured by patients) and on lipid levels (total cholesterol, HDL, LDL, HDL:LDL ratio, triglycerides) have been assessed for Cohorts A (100 mg), B (200 mg) and C (400 mg). The data from these studies are presented in the following tables as actual change from baseline measurement, and percent change from baseline measurement, during Week 6, and during Week 10, as indicated. Shown in the tables is the number of evaluated data points (n), the mean value and standard deviation (std) for each treatment group, and the minimum and maximum measurement for each treatment group. Measurements at Week 6 occur after 8 doses of ISIS 113715 (3 doses during week 1 and one each during weeks 2-6). Measurements at Week 10 occur 4 weeks after last dose. A negative number indicates a decrease from baseline or screen, while a positive number indicates an increase from baseline or screen. Data from these studies are also presented in figures incorporated herein. For the figures containing data from CS-7, the differences between ISISI 13715-treated groups and placebo were estimated using ANCOVA, with screening or baseline measure included as the covariate in the model. The simultaneous 95% confidence intervals were constructed using the Dunnett's method.

Shown in table 6a are measurements of HbA_{1c} changes at Week 6 and Week 10. Baseline HbA_{1c} levels were not measured for the 100 mg Cohort, but the protocol was amended in time to take the measurement for patients in Cohort C and a few patients in Cohort B.

TABLE 6a
Changes in HbA_{1c} levels at Week 6 and Week 10

| | Measure | Statistic | Placebo | 100 mg | 200 mg | 400 mg |
|---|---|---|---|---|---|---|
| Week 6 | Actual change | n | 4 | 0 | 2 | 11 |
| | from baseline | mean | −0.4 | — | −0.6 | −0.7 |
| | | std | 0.4 | — | 0.1 | 0.5 |
| | | min | −0.9 | — | −0.7 | −1.5 |
| | | max | −0.1 | — | −0.5 | 0.0 |
| | % change from | n | 4 | 0 | 2 | 11 |
| | baseline | mean | −5.8 | — | −8.0 | −8.5 |
| | | std | 5.3 | — | 2.3 | 5.5 |
| | | min | −12.7 | — | −9.6 | −16.9 |
| | | max | −1.3 | — | −6.4 | 0.0 |
| Week | Actual change | n | 4 | 0 | 2 | 12 |
| 10 | from baseline | mean | −0.3 | — | −1.1 | −0.8 |
| | | std | 0.2 | — | 0.1 | 0.7 |
| | | min | −0.5 | — | −1.2 | −1.8 |
| | | max | −0.2 | — | −1.0 | 0.3 |
| | % change from | n | 4 | 0 | 2 | 12 |
| | baseline | mean | −3.8 | — | −14.6 | −9.5 |
| | | std | 2.2 | — | 2.5 | 8.2 |
| | | min | −7.0 | — | −16.4 | −22.2 |
| | | max | −2.6 | — | −12.8 | 2.7 |

As shown in Table 6a, on average, patients treated with ISIS 113715 exhibited greater changes in HbA_{1c} levels than placebo treated patients at Week 6, and the effect was still apparent at Week 10, suggesting protracted effects.

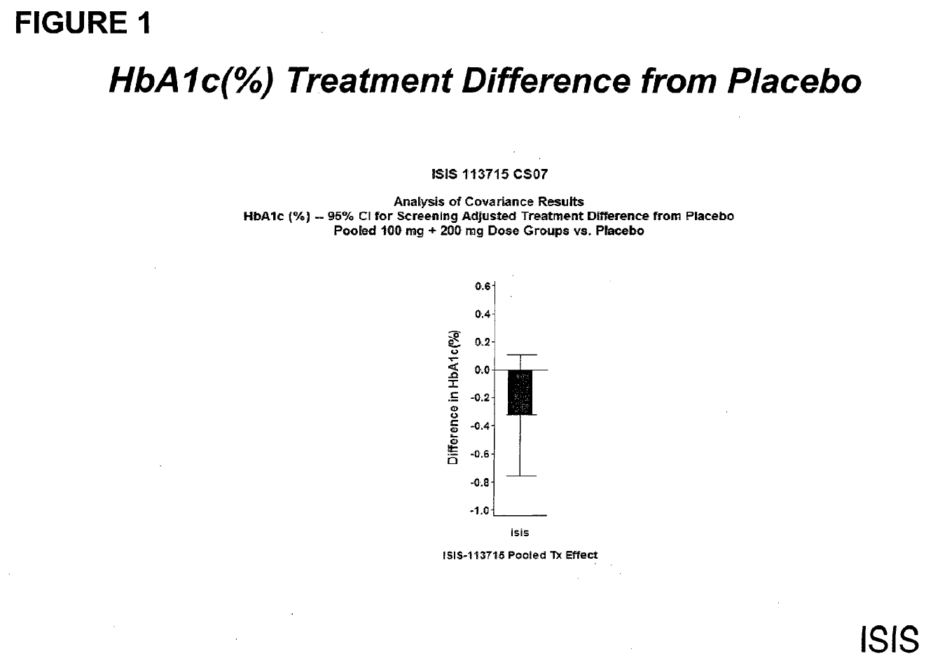

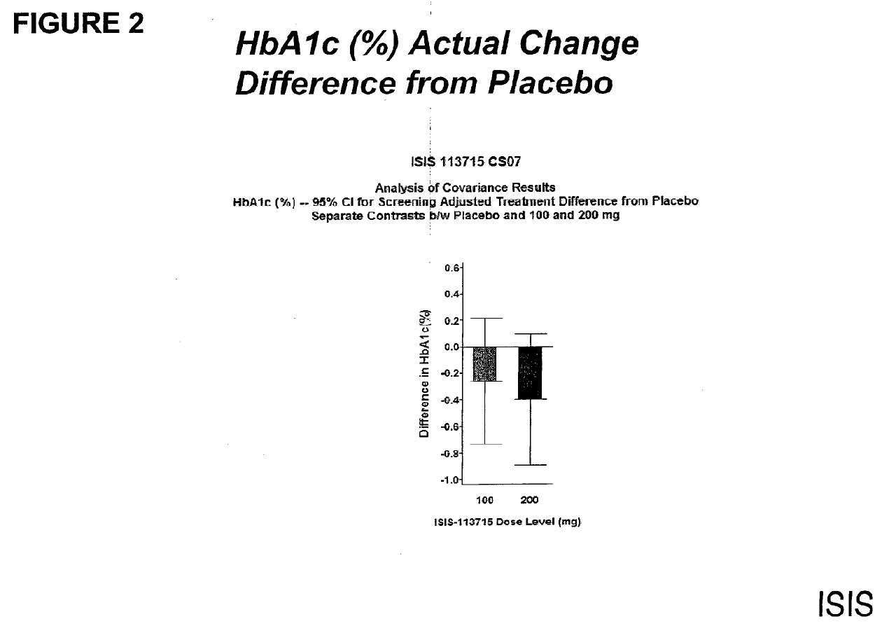

HbA_{1c} levels were measured for patients in Cohorts A and B during the screening period, and shown in FIG. 1 are the analysis of covariance results for screening adjusted treatment differences between pooled 100 mg and 200 mg cohorts versus placebo at Week 6. FIG. 2 depicts screening adjusted differences from placebo for the 100 mg and 200 mg cohorts separately at Week 6. As with the comparisons to baseline levels shown in Table 6a, these data show that treatment with ISIS 113715 causes reductions in HbA_{1c} levels in Type 2 diabetics. There is a strong correlation between levels of HbA_{1c} and the average blood glucose levels over the previous 3 months, and thus decreases in HbA_{1c} are indicative of sustained blood glucose control. The reductions over just 6 weeks of treatment are therefore promising.

Shown in Table 6b are HbA_{1c} levels (%) for individual patients in the placebo group, Cohort A (100 mg), Cohort B (200 mg), and Cohort C (400 mg) at screening, baseline (if measured), and at Week 6 and Week 10.

TABLE 6b
Individual Patient HbA_{1c} levels at Screen, Baseline,
Week 6 and Week 10

| Treatment | | | | | |
|---|---|---|---|---|---|
| group | Subject | Screen | Baseline | W6 | W10 |
| Placebo | 1 | 7.0 | — | 7.0 | 6.8 |
| | 2 | 10.0 | — | 9.5 | 9.8 |
| | 3 | 7.5 | — | 6.4 | 6.2 |
| | 4 | 8.3 | — | 8.1 | 8.0 |
| | 5 | 9.9 | — | 8.4 | 8.3 |
| | 6 | 9.8 | — | 8.0 | 7.9 |
| | 7 | 8.1 | 7.7 | 7.6 | 7.5 |
| | 8 | 8.0 | 7.8 | 7.7 | 7.6 |
| | 9 | 7.2 | — | 6.2 | 6.2 |
| | 10 | 9.8 | 6.9 | 6.4 | 6.7 |
| | 11 | 7.0 | — | 6.6 | 6.6 |
| | 12 | 9.0 | 7.1 | 6.2 | 6.6 |
| 100 mg | 13 | 8.2 | — | 7.8 | 7.4 |
| | 14 | 6.9 | — | 6.0 | 6.1 |
| | 15 | 7.2 | — | 6.5 | 6.5 |
| | 16 | 8.0 | — | 6.6 | 6.7 |
| | 17 | 9.2 | — | 7.9 | 7.8 |
| | 18 | 6.9 | — | 6.5 | 6.8 |
| | 19 | 8.6 | — | 7.6 | 7.7 |
| | 20 | 9.0 | — | 8.4 | 8.2 |
| | 21 | 9.0 | — | 8.0 | 8.0 |
| | 22 | 6.9 | — | 6.2 | 6.0 |
| | 23 | 7.1 | — | 6.1 | 6.2 |
| | 24 | 8.9 | — | 8.3 | 8.0 |
| 200 mg | 25 | 6.7 | — | 6.1 | 6.3 |
| | 26 | 7.9 | — | 7.6 | 7.6 |
| | 27 | 7.0 | — | 6.2 | 6.1 |
| | 28 | 8.5 | — | 6.4 | — |
| | 29 | 7.2 | — | 6.7 | 7.0 |
| | 30 | 8.0 | — | 7.8 | 8.4 |
| | 31 | 9.2 | — | 7.4 | 7.4 |
| | 32 | 7.6 | — | 6.3 | 6.4 |
| | 33 | 7.5 | — | 7.1 | 7.8 |
| | 34 | 8.2 | 7.3 | 6.6 | 6.1 |
| | 35 | 7.7 | — | 7.6 | — |
| | 36 | 8.0 | 7.8 | 7.3 | 6.8 |
| 400 mg | 37 | 8.4 | 8.2 | 8.8 | 9.0 |
| | 38 | 9.5 | 8.0 | 7.3 | 7.6 |
| | 39 | 8.6 | 9.5 | 8.5 | 8.2 |
| | 40 | 9.4 | 9.1 | 9.1 | 9.4 |
| | 41 | 10.6 | 10.3 | 8.9 | 8.8 |
| | 42 | 11.8 | 11.3 | 10.5 | 10.3 |
| | 43 | 9.6 | 8.1 | 6.7 | 6.3 |
| | 44 | 8.9 | 8.5 | 7.5 | 6.9 |
| | 45 | 6.8 | 6.6 | 6.3 | 6.3 |
| | 46 | 7.3 | 8.5 | — | — |
| | 47 | 6.8 | 6.7 | 6.4 | 6.5 |
| | 48 | 7.9 | 7.5 | 7.0 | 7.1 |
| | 49 | 7.9 | 6.9 | 6.0 | 5.5 |
| | 50 | 8.9 | 7.7 | 7.6 | 7.7 |

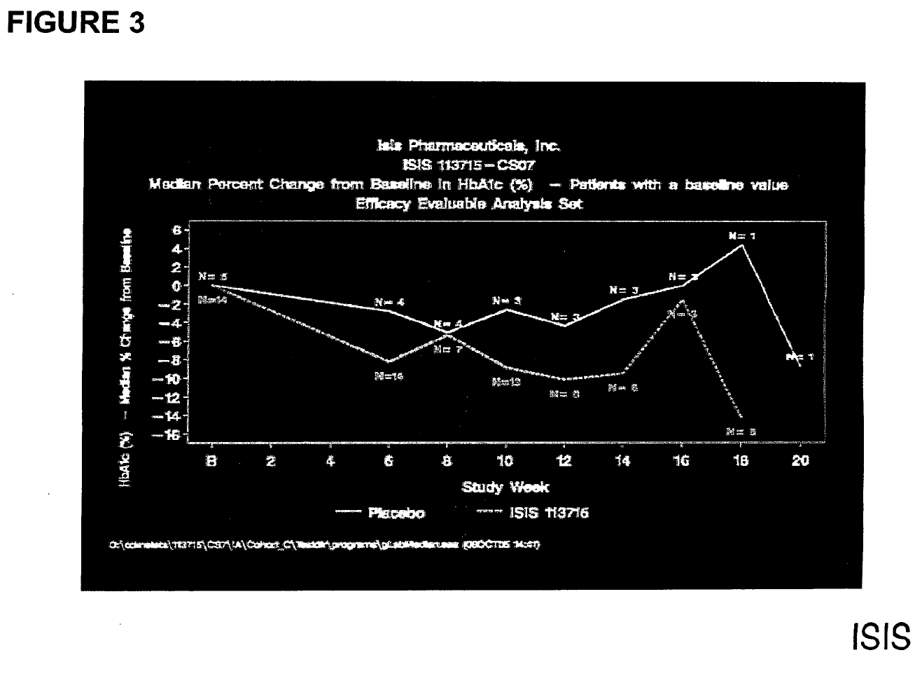

The median percent change from baseline in HbA_{1c} % for Cohort C as compared to placebo is shown in FIG. 3. Although there is an initial decline in median HbA_{1c} levels in the placebo group, this trend appears to level off after about Week 6, while the more dramatic decline observed in the treatment group continues the downward trend out to Week 14. For this analysis, the weeks were calculated from the first dose date, in two-week windows. These data support protracted reduction of HbA_{1c} with ISIS 113715 treatment.

Shown in Table 7 are the changes in fasting serum glucose levels measured at Week 6 and at Week as compared to baseline levels.

TABLE 7
Changes in fasting serum glucose (mg/dL) at
Week 6 and Week 10

| | | Statistic | Placebo | 100 mg | 200 mg | 400 mg |
|---|---|---|---|---|---|---|
| Week 6 | Actual change | n | 12 | 12 | 10 | 12 |
| | from baseline | mean | −10 | 0 | −2 | −4 |
| | | std | 25 | 19 | 16 | 33 |
| | | min | −61 | −21 | −24 | −44 |
| | | max | 30 | 35 | 21 | 79 |
| | % change from | n | 12 | 12 | 10 | 12 |
| | baseline | mean | −5 | 1 | −1 | −2 |
| | | std | 15 | 12 | 11 | 18 |
| | | min | −29 | −11 | −15 | −19 |
| | | max | 24 | 24 | 15 | 44 |
| Week | Actual change | n | 12 | 12 | 10 | 12 |
| 10 | from baseline | mean | 7 | 2 | 0 | −8 |
| | | std | 25 | 25 | 25 | 42 |
| | | min | −27 | −25 | −25 | −54 |
| | | max | 56 | 53 | 60 | 110 |
| | % change from | n | 12 | 12 | 10 | 12 |
| | baseline | mean | 4 | 3 | 0 | −4 |
| | | std | 14 | 18 | 17 | 23 |
| | | min | −15 | −14 | −18 | −27 |
| | | max | 29 | 39 | 42 | 62 |

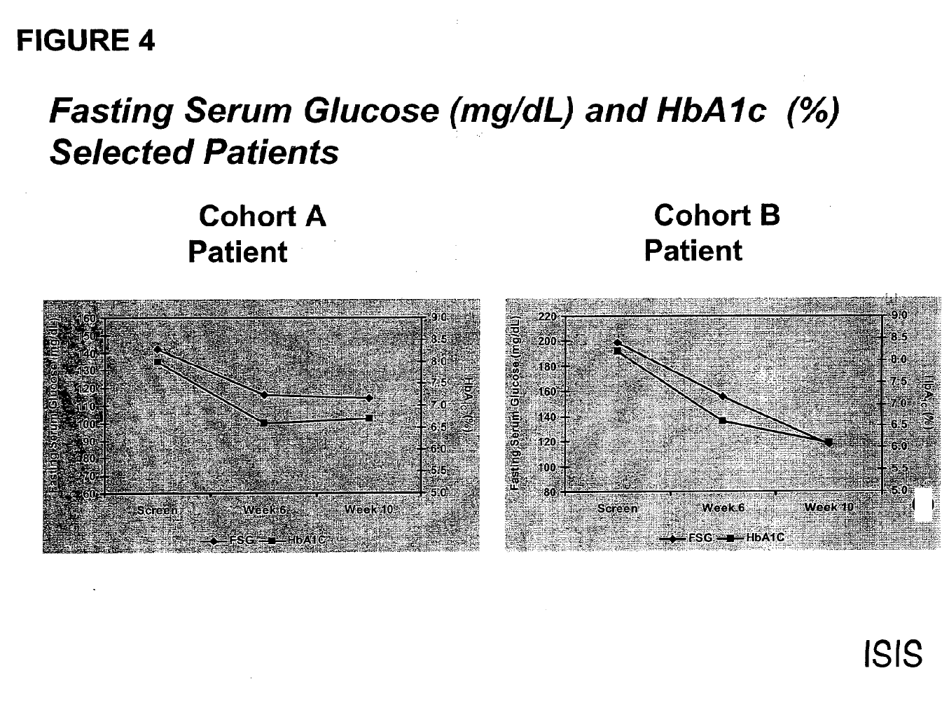

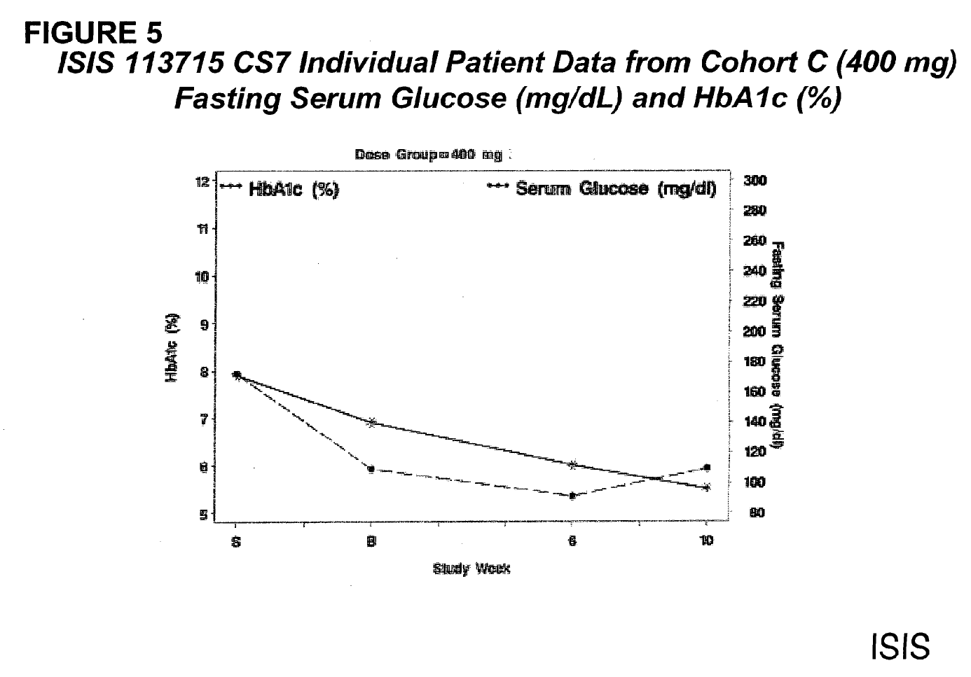

As shown in Table 7, at week 10, there is a trend toward reductions in fasting serum glucose with treatment with ISIS 113715 in patients with Type 2 diabetes. FIGS. 4 and 5 show data from individual patients in Cohorts A, B, and C, depicting parallel reductions in HbA_{1c} levels and fasting serum glucose levels.

Shown in Table 8 are the average changes in the averaged daily fasting blood glucose levels measured by patients as compared to baseline levels. Measurements are averaged weekly for each patient. Mean data for each treatment group processed in this manner is shown.

TABLE 8
Average Blood Glucose (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 10 | 11 |
| | from baseline | mean | −5 | 0 | 0 | −10 |
| | | std | 11 | 17 | 20 | 20 |
| | | min | −21 | −37 | −34 | −48 |
| | | max | 9 | 26 | 32 | 31 |
| | % change from | n | 12 | 12 | 10 | 11 |
| | baseline | mean | −3 | 0 | 1 | −7 |
| | | std | 7 | 11 | 14 | 11 |
| | | min | −14 | −21 | −22 | −25 |
| | | max | 7 | 19 | 27 | 15 |
| Week | Actual change | n | 11 | 12 | 10 | 12 |
| 10 | from baseline | mean | −3 | 3 | 3 | −8 |
| | | std | 16 | 21 | 23 | 14 |
| | | min | −22 | −35 | −29 | −25 |
| | | max | 32 | 48 | 52 | 27 |
| | % change from | n | 11 | 12 | 10 | 12 |
| | baseline | mean | −2 | 2 | 3 | −5 |
| | | std | 9 | 14 | 18 | 8 |
| | | min | −11 | −20 | −20 | −17 |
| | | max | 17 | 32 | 40 | 13 |

For Cohort C, there is a more pronounced reduction from baseline m the 400 mg treatment group as compared to placebo control, consistent with management of hyperglycemia in the patients treated with ISIS 113715.

Shown in Table 9 are changes in fasting plasma glucose levels at Week 6.

TABLE 9
Fasting Plasma Glucose levels (mg/dL) at Week 6

| | | Statistic | Placebo | 100 mg | 200 mg | 400 mg |
|---|---|---|---|---|---|---|
| Week 6 | Actual change | n | 10 | 12 | 10 | 11 |
| | from baseline | mean | 15 | −3 | −4 | −2 |
| | | std | 63 | 22 | 14 | 30 |
| | | min | −45 | −22 | −22 | −46 |
| | | max | 182 | 47 | 19 | 71 |
| | % change from | n | 10 | 12 | 10 | 11 |
| | baseline | mean | 8 | −1 | −2 | −1 |
| | | std | 32 | 15 | 10 | 18 |
| | | min | −21 | −16 | −15 | −20 |
| | | max | 91 | 33 | 16 | 44 |

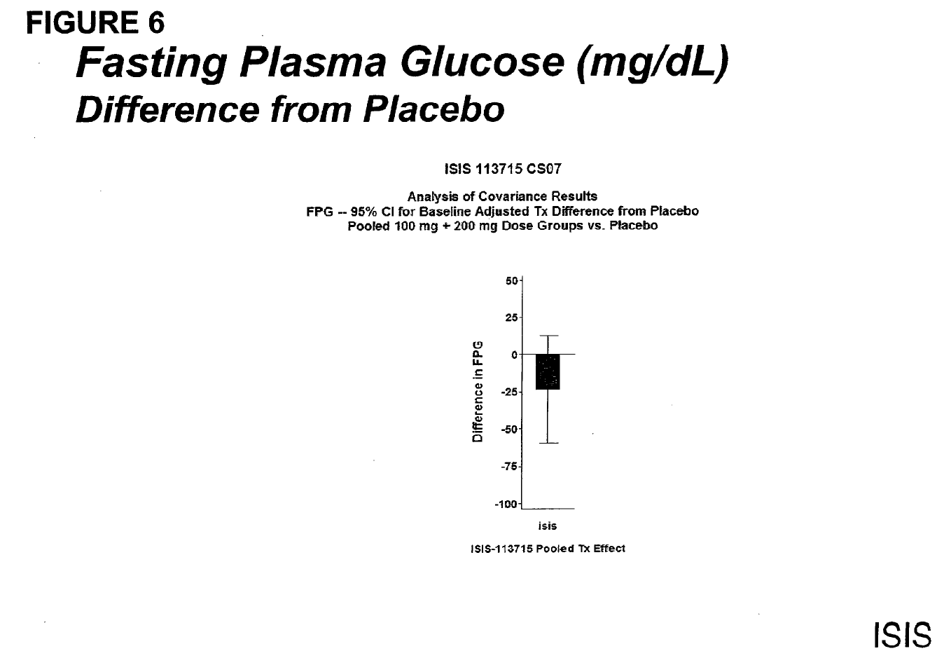

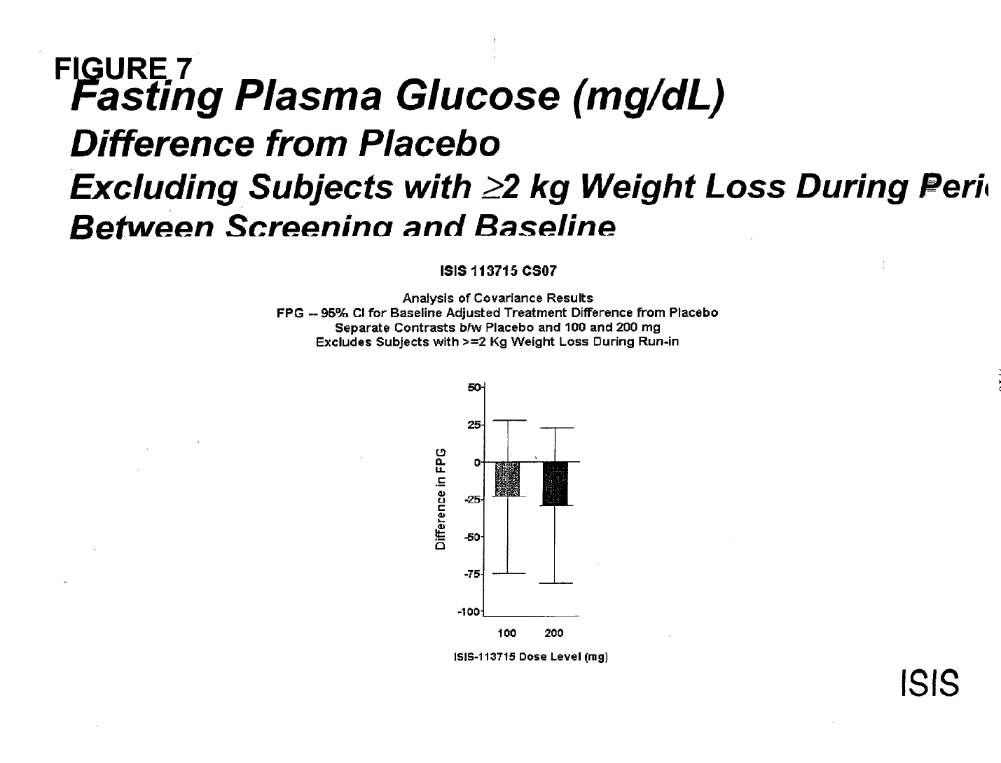

As shown in Table 9, treatment with ISIS 113715 caused a more pronounced reduction from baseline as compared to placebo. FIGS. 6 and 7 likewise depict reductions in fasting plasma glucose in the analysis of covariance results for the baseline adjusted treatment groups as compared to control. As shown in FIG. 5, treatment with ISIS 113715 on average reduced fasting plasma glucose levels by about 25 mg/dL.

Tables 10 to 15 show changes in lipid levels from baseline measurements at Week 6 and Week 10.

TABLE 10
Cholesterol levels (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 3 | −15 | −11 | −4 |
| | | std | 14 | 35 | 15 | 24 |
| | | min | −21 | −81 | −26 | −51 |
| | | max | 27 | 45 | 26 | 22 |
| | % change from | n | 12 | 12 | 11 | 12 |
| | baseline | mean | 2 | −4 | −5 | −1 |
| | | std | 6 | 16 | 11 | 12 |
| | | min | −8 | −23 | −14 | −23 |
| | | max | 12 | 27 | 26 | 14 |
| Week | Actual change | n | 12 | 12 | 11 | 12 |
| 10 | from baseline | mean | 4 | 3 | −5 | −8 |
| | | std | 29 | 39 | 17 | 22 |
| | | min | −28 | −65 | −32 | −66 |
| | | max | 73 | 72 | 23 | 20 |
| | % change from | n | 12 | 12 | 11 | 12 |
| | baseline | mean | 3 | 3 | −2 | −3 |
| | | std | 14 | 18 | 10 | 10 |
| | | min | −11 | −27 | −14 | −24 |
| | | max | 38 | 37 | 23 | 14 |

TABLE 11
HDL levels (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 0 | 0 | 0 | 5 |
| | | std | 5 | 6 | 4 | 9 |
| | | min | −5 | −10 | −6 | −6 |
| | | max | 9 | 7 | 5 | 21 |
| | % change from | n | 12 | 12 | 11 | 12 |
| | baseline | mean | 1 | 2 | 1 | 11 |
| | | std | 11 | 13 | 9 | 19 |
| | | min | −12 | −20 | −14 | −13 |
| | | max | 25 | 25 | 13 | 38 |
| Week | Actual change | n | 12 | 12 | 11 | 12 |
| 10 | from baseline | mean | 2 | 8 | 3 | 6 |
| | | std | 7 | 8 | 6 | 11 |
| | | min | −8 | −8 | −4 | −10 |
| | | max | 16 | 22 | 14 | 35 |
| | % change from | n | 12 | 12 | 11 | 12 |
| | baseline | mean | 7 | 19 | 8 | 14 |
| | | std | 17 | 19 | 16 | 23 |
| | | min | −12 | −15 | −11 | −22 |
| | | max | 44 | 47 | 39 | 64 |

TABLE 12
LDL levels (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 1 | −12 | −6 | −7 |
| | | std | 16 | 23 | 15 | 13 |
| | | min | −35 | −58 | −23 | −34 |
| | | max | 23 | 26 | 21 | 13 |
| | % change from | n | 12 | 12 | 11 | 12 |
| | baseline | mean | 2 | −7 | −3 | −4 |
| | | std | 9 | 16 | 15 | 10 |
| | | min | −17 | −38 | −18 | −23 |
| | | max | 14 | 23 | 33 | 14 |
| Week | Actual change | n | 12 | 12 | 11 | 12 |
| 10 | from baseline | mean | 5 | 3 | −12 | −17 |
| | | std | 20 | 25 | 18 | 13 |
| | | min | −25 | −43 | −39 | −47 |
| | | max | 40 | 48 | 16 | 6 |
| | % change from | n | 12 | 12 | 11 | 12 |
| | baseline | mean | 4 | 4 | −8 | −12 |
| | | std | 13 | 17 | 16 | 8 |
| | | min | −12 | −26 | −32 | −23 |
| | | max | 27 | 33 | 25 | 7 |

TABLE 13
VLDL levels (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 2 | −3 | −5 | −2 |
| | | std | 19 | 23 | 15 | 8 |
| | | min | −34 | −56 | −43 | −12 |
| | | max | 46 | 29 | 10 | 12 |
| | % change from | n | 11 | 12 | 11 | 11 |
| | baseline | mean | 10 | 6 | −2 | −7 |
| | | std | 83 | 61 | 52 | 78 |
| | | min | −100 | −65 | −94 | −75 |
| | | max | 180 | 120 | 100 | 171 |
| Week | Actual change | n | 11 | 12 | 10 | 12 |
| 10 | from baseline | mean | −5 | −8 | 4 | 3 |
| | | std | 11 | 22 | 9 | 8 |
| | | min | −29 | −48 | −4 | −9 |
| | | max | 11 | 42 | 27 | 21 |
| | % change from | n | 10 | 12 | 10 | 11 |
| | baseline | mean | −13 | −27 | 17 | 31 |
| | | std | 72 | 60 | 38 | 64 |
| | | min | −100 | −92 | −24 | −63 |
| | | max | 140 | 89 | 100 | 167 |

TABLE 14
HDL:LDL ratios at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 0 | 0.04 | 0.02 | 0.06 |
| | | std | 0.04 | 0.07 | 0.06 | 0.07 |
| | | min | −0.06 | −0.03 | −0.08 | −0.01 |
| | | max | 0.07 | 0.20 | 0.15 | 0.20 |
| | % change from | n | 12 | 12 | 11 | 12 |
| | baseline | mean | 0.2 | 12.6 | 6.8 | 15.9 |
| | | std | 12 | 25.1 | 16 | 13.7 |
| | | min | −16.2 | −14 | −15.3 | −5.8 |
| | | max | 18.3 | 79 | 30.3 | 38.7 |
| Week | Actual change | n | 12 | 12 | 11 | 12 |
| 10 | from baseline | mean | −0.01 | 0.05 | 0.07 | 0.12 |
| | | std | 0.04 | 0.05 | 0.11 | 0.15 |
| | | min | −0.05 | −0.03 | −0.05 | 0.00 |
| | | max | 0.05 | 0.17 | 0.34 | 0.56 |
| | % change from | n | 12 | 12 | 11 | 12 |
| | baseline | mean | 2.8 | 15.9 | 20.4 | 29 |
| | | std | 11.6 | 15.6 | 30.2 | 24.8 |
| | | min | −17.2 | −8.5 | −11.8 | 1.1 |
| | | max | 13.7 | 50.1 | 90.2 | 85.7 |

TABLE 15
Triglyceride levels (mg/dL) at Week 6 and Week 10

| | | Statistic | Placebo | 100 mg | 200 mg | 400 mg |
|---|---|---|---|---|---|---|
| Week 6 | Actual change | n | 12 | 12 | 11 | 12 |
| | from baseline | mean | 5 | 14 | −59 | −18 |
| | | std | 78 | 111 | 109 | 40 |
| | | min | −108 | −114 | −367 | −77 |
| | | max | 188 | 276 | 34 | 62 |
| | % change from | n | 12 | 12 | 11 | 12 |
| | baseline | mean | 10 | 2 | −17 | −8 |
| | | std | 48 | 38 | 26 | 28 |
| | | min | −51 | −42 | −74 | −38 |
| | | max | 97 | 80 | 32 | 58 |
| Week | Actual change | n | 12 | 12 | 11 | 12 |
| 10 | from baseline | mean | −28 | −5 | 0 | −1 |
| | | std | 51 | 107 | 112 | 61 |
| | | min | −105 | −142 | −218 | −91 |
| | | max | 51 | 267 | 261 | 129 |
| | % change from | n | 12 | 12 | 11 | 12 |
| | baseline | mean | −9 | −1 | 1 | −1 |
| | | std | 34 | 38 | 28 | 40 |
| | | min | −51 | −52 | −44 | −63 |
| | | max | 59 | 75 | 47 | 86 |

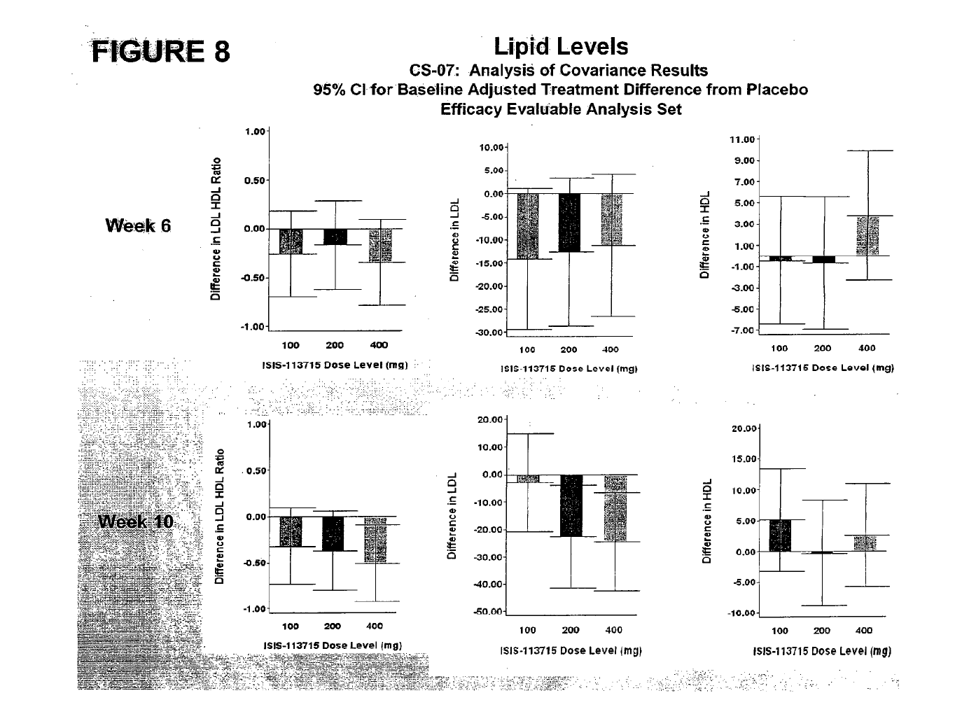

A survey of the mean changes in lipid levels depicted in Tables 10 to 15 show decreases in cholesterol, LDL levels, VLDL levels, and triglycerides and increases in HDL levels and HDL:LDL ratio in the groups treated with ISIS 113715, and lipid alterations are present at Week 6 as well as Week 10. Shown in FIG. 8 are the analysis of covariance results for baseline adjusted treatment differences from placebo for the lipid parameters separated out by dose cohort. These results show that treatment with ISIS 113715 alters lipid levels and, consequently, lipid profile, in patients with Type 2 diabetes.

### Example 28

### Human Clinical Trials—Phase I, Low Dose Subcutaneous Administration to Normal Subjects (CS-8)

A follow-on study to CS-3 was conducted to evaluate the safety and tolerability of administering ISIS 113715 subcutaneously in daily low doses and to evaluate the plasma bioavailability of ISIS 113715. Twenty normal volunteers in two cohorts received drug. Cohort A received subcutaneous injections of ISIS 113715, 15 mg/day for 10 consecutive days. Cohort B received subcutaneous injections of ISIS 113715, 30 mg/day for 10 consecutive days. Follow up was one additional week (days 11-17). Endpoints of study are safety and tolerability and pharmacokinetics. Preliminary pharmacokinetic analyses indicate reproductible and dose-dependent exposure by the subcutaneous route at the 15 and 30 mg per injection dose. Cₘₐₓ concentrations were 0.128 μg/mL and 0.320 μg/mL following single dose administration of 15 and 30 mg, respectively. Mean tₘₐₓ ranged from 2. to 3.7 hours after s.c. injection. By 24 hours after a single injection, plasma concentrations had decreased 50 to 100-fold less than Cₘₐₓ. In addition, after 10 consecutive daily s.c. doses, pharmacokinetics was not altered and did not exhibit accumulation in plasma based on Cₘₐₓ and AUC measurements. Mean plasma bioavailability (% F) was estimated to be between 32 and 46% based on historical i.v. plasma AUC (6.15 μg·h/mL at a dose of 32.4 mg (0.5 mg/kg).

### Example 29

### Solid Dosage Formulations for Clinical Evaluation

The purpose of this study is to clinically evaluate PEG-based immediate releasing and pulsatile formulations for enhanced oral oligonucleotide absorption by way of rapidly producing and further extending the dynamic action of sodium caprate (C10) by releasing an additional amount of C10 after the initial amount. Pulsatile release formulations are described in published US Patent Application Publication No. 2003/0124196, the contents of which are incorporated by reference herein in their entirety. Three types of dosage forms, representing four formulations, are evaluated in humans:

1. Enteric coated (EC) capsules comprising a single population of immediate releasing (IR) 2 mm minitablets with the full doses of oligonucleotide and C10;

2. EC monolithic tablets comprising the full doses of oligonucleotide and C10; and

3. EC pulsed-release capsules comprising both a mixture of IR 2 mm minitablets with the full dose of oligonucleotide and partial dose of C10, and delayed release 2 mm minitablets having the remainder of the C10 dose and lacking oligonucleotide.

The immediate releasing components of the above three dosage forms (4 formulated batches) are made from, for example, hot-melt granulations of PEG-3350, ISIS 113715 and sodium caprate in a high shear mixer, preferably with a controlled temperature of about 70° C. The granules may be compressed into tablets or minitablets without the use of additional excipients.

Two approaches are intended for the delayed release (pulsed C10) minitablets. It is believed that a matrixed polymer has a typical burst release of C10 followed by a sustained release over a designated time.

A coated polymer approach is characterized by a lag time with more of a delayed (bolus release) profile rather than that expected from a sustained release. Both of these approaches are pursued in order to effectively bracket the two parameters mentioned in dosage form 3 above, that is, the delay time and fractional amount of C10 to be released. The C10 released from the matrix burst is actually construed as part of the initially released C10 pulse—from the other population of minitablets in the capsule (the IR formulation). This consideration of additional initial C10 is important in view of the perceived minimum threshold of dissolved C10 required for permeability enhancement. Accordingly, the appropriate populations of minitablets are filled into Size 00 capsules and then banded prior to enteric coating with HPMC-50.

Tables 16 and 17 detail four sample formulations.

TABLE 16
ISIS 113715 Immediate Release Sample Formulations

| | Formulation 1 | Formulation 2 |
|---|---|---|
| Characteristic | Control IR Minitablet* | Monolithic IR Tablet* |
| Target dose per capsule | 100:500 mg | 100:500 mg |
| ISIS 113715:C10 | | |
| Composition (max wt ~700 mg) | Mg | mg |
| C10 - Sodium Caprate | 500 | 500 |
| ISIS 113715 | 100 | 100 |
| PEG3350 | Tbd | tbd |
| Manufacture Process | Hot-melt granulation | Hot-melt granulation |
| | Compression - 2 mm | Compression - oval caplets |
| | Encapsulation (Size 00) & EC | Enteric Coating |
| Physical and Analytical Testing | Assay, Content Uniformity, | Assay, Content Uniformity, |
| | Disintegration (acid, neutral), | Disintegration (acid, neutral), |
| | Dissolution | Dissolution |

*One granule batch will be used for formulations 1 and 2

TABLE 17
ISIS 113715 Pulsed Minitablet Sample GMP Formulations

| | Formulation 3 - matrix pulse | | | |
|---|---|---|---|---|
| | | Pulsed | Formulation 4 - coated pulse | |
| | IR minitab* | minitab | IR minitab* | Pulsed minitab |
| Characteristic | details | details | details | details |
| Target dose per capsule | 100:225 mg | 0:275 mg | 100:225 mg | 0:275 mg |
| ISIS 113715:C10 | | | | |
| Composition (max wt ~700 mg) | mg | mg | mg | mg |
| C10 - Sodium Caprate | 225 | 275 | 225 | 275 |
| ISIS 113715 Full Length Purity | 100 | — | 100 | — |
| PEG3350 | tbd | tbd | tbd | tbd |
| Polymer (tbd) | — | tbd | — | tbd |
| Manufacture Process | Hot-melt gran | Hot-melt gran | Hot-melt gran | Hot-melt gran |
| (Followed by encapsulation into | Compression | (intra | Compression | Compression |
| Size 00 caps and enteric coating) | | polymer) | | Apply Coating |
| | | Compression | | |
| Physical and Analytical Testing | Dissolution | Dissolution | Dissolution | Dissolution |
| | Assay | | Assay | |

| | | |
|---|---|---|
| Physical and Analytical Testing | Assay, Content Uniformity, | Assay, Content Uniformity, |
| | Disintegration (acid, neutral), | Disintegration (acid, neutral), |
| | Dissolution | Dissolution |

*The IR minitablets for the two types of formulations are identical and will be made as a single batch. (tbd = to be determined from development work)

The pharmaceutical formulations described above may be administered as a single (e.g., 200 mg oligonucleotide in a single tablet) or divided (e.g., 2×100 mg oligonucleotide tablets taken at the same time) oral dose once per day in an amount comprising between about 50 mg and 1,000 mg oligonucleotide, preferably between about 100 mg and 500 mg oligonucleotide, and more preferably between about 100 and 200 mg oligonucleotide. Alternatively, the total dosage may be divided and administered as separate dosages two, three or more times per day (i.e., one 100 mg tablet twice per day).

### Example 30

### Combination Therapy Using ISIS 113715 and Rosiglitazone in Aged ZDF Rats

Rosiglitazone (Avandia™; GlaxoSmithline) is a member of the thiazolidinedione (TZD) class of insulin sensitizers. It is an accepted treatment for Type 2 diabetes, either as monotherapy or in combination with sulfonylureas, insulin, or metformin. It increases insulin sensitivity in muscle, liver and fat tissues. Because rosiglitazone can cause fluid retention, it must be used with caution in patients with edema or at risk for heart failure. Rosiglitazone also causes weight gain in a dose-dependent manner.

A combination of ISIS 113715 and rosiglitazone was administered to aged, very insulin-resistant ZDF rats, aged approximately 15 weeks at start of study (in contrast, ZDF rats in previous studies are 6-10 weeks of age at start of study). These aged animals have little insulin by this age and thus do not respond to maximal doses of ISIS 113715 or rosiglitazone alone.

Aged ZDF rats were given ISIS 113715 by intraperitoneal injection at doses of 25 mg/kg twice a week, and rosiglitazone 3 mg/kg/day orally (in food) for three weeks. Plasma glucose was measured at week 0 (before treatment) and after weeks 1, 2 and 3 of treatment. Neither rosiglitazone or ISIS 113715 alone produced a significant reduction in blood glucose at any time point over the three weeks compared to saline or negative-control oligonucleotide (ISIS 141923)-treated rats. A combination of ISIS 141923 (25 mg/kg) and rosiglitazone (3 mg/kg/day) also had no effect. All of these groups had blood glucose levels of approx 400-460 mg/dL.

However, the combination of ISIS 113715 and rosiglitazone decreased blood glucose at week 1 to approximately 320 mg/dL, at week 2 to approximately 310 mg/dL and at week 3 to approximately 230 mg/dL.

These rats were weighed at week 2 and week 3 of treatment. Saline treated rats gained an average of approximately 11 grams after week 2 and 8 grams after week 3. The expected increased weight gain was observed in rosiglitazone-treated animals (weight gain of approximately 32 gm after week 2 and 40 gm after week 3), but this was not significantly increased or decreased by combination treatment with ISIS 113715 (weight gain of approximately 39 gm after week 2 and 43 gm after week 3 for rosiglitazone plus ISIS 113715 treatment). ISIS 113715 alone did not cause significant weight gain (weight gain of approximately 17 gm after week 2 and 9 gm after week 3).

Thus combination therapy with ISIS 113715 does not compound the side effects on body weight observed with rosiglitazone alone.

AST/ALT and plasma cholesterol levels were measured in the rats at weeks 1, 2, 3, 4 and 5, but no significant effects were seen in any treatment group (saline, control oligonucleotide ISIS 141923, antisense to PTP1B ISIS 113715, rosiglitazone alone, rosiglitazone plus ISIS 141923 and rosiglitazone plus ISIS 113715).

Insulin tolerance tests (ITT) were conducted in the rats at week 3. Insulin (1.5 U/Kg in PBS @ 3 U/mL) was injected intraperitoneally and plasma glucose was measured over time. The results were graphed and the area under the curve (AUC, expressed in mg/dL×min) is a measure of insulin sensitivity. These “old” ZDF rats are normally very resistant to insulin (large AUC).

Saline treated rats had an average AUC of approximately 18,600 mg/dL×min. Rats treated with negative control oligonucleotide (ISIS 141923), antisense to PTP1B (ISIS 113715), or rosiglitazone had similar average AUCs of approximately 13,800-14,600 mg/dL×min. Rats treated with a combination of rosiglitazone and ISIS 113715 had an average AUC of approximately 7500, a reduction of nearly 60%.

This indicates a significant and synergistic increase in insulin sensitivity when rosiglitazone is combined with ISIS 113715, the antisense inhibitor of PTP1B. No hypoglycemia resulted from the combination of rosiglitazone and ISIS 113715 even at high doses of injected insulin.

### Example 31

### Combination Therapy Using ISIS 113715 and Metformin in ZDF Rats

Metformin (Glucophage™) is an accepted treatment for Type 2 diabetes, either as monotherapy or in combination with sulfonylureas, insulin or rosiglitazone. It improves glucose tolerance and insulin sensitivity by increasing peripheral glucose uptake and utilization. Metformin is contraindicated in patients with congestive heart failure. Lactic acidosis, a buildup of lactic acid in the blood, is also a known side effect of metformin treatment. While rare (one in 33,000 patients), it can be fatal in up to half the patients who develop it.

A combination of ISIS 113715 and metformin was administered to ZDF rats.

Ten-week old ZDF rats were given ISIS 113715 by intraperitoneal injection at 12.5 mg/kg twice a week (ED₂₀ dose) and metformin by oral gavage at 100, 300 or 500 mg/kg per day for four weeks. 500 mg/kg is the maximally effective dose that can be tolerated by these rats. Plasma glucose was measured at week 0 (before treatment) and after weeks 1, 2 and 4 of treatment. Results are shown in Table 18.

TABLE 18
Effects of Metformin and ISIS 113715 Combination
in Zucker Diabetic Fatty Rats

| | Time: | | | | |
|---|---|---|---|---|---|
| | Week 0 | Week 1 | Week 2 | Week 3 | Week 4 |
| Compound | Plasma glucose mg/dL ± SEM | | | | |
| Saline | 253 ± 32 | 472 ± 22 | 496 ± 33 | 543 ± 20 | 569 ± 43 |
| PTP1B | 257 ± 52 | 394 ± 75 | 415 ± 58 | 396 ± 60 | 407 ± 57 |
| 113715 | | | | | |
| 113715 + | 255 ± 32 | 348 ± 24 | 391 ± 16 | 345 ± 26 | 363 ± 31 |
| Met 100 | | | | | |
| 113715 + | 254 ± 43 | 276 ± 54 | 370 ± 60 | 373 ± 47 | 361 ± 44 |
| Met 300 | | | | | |
| 113715 + | 249 ± 42 | 241 ± 57 | 285 ± 43 | 320 ± 43 | 258 ± 47 |
| Met 500 | | | | | |
| Metformin | 256 ± 29 | 428 ± 20 | 504 ± 20 | 494 ± 18 | 393 ± 71 |
| 100 mg/kg | | | | | |
| Metformin | 251 ± 27 | 323 ± 33 | 415 ± 29 | 449 ± 11 | 448 ± 42 |
| 300 mg/kg | | | | | |
| Metformin | 252 ± 28 | 244 ± 56 | 344 ± 59 | 374 ± 53 | 443 ± 35 |
| 500 mg/kg | | | | | |

The effect of metformin alone was lost over time as the diabetic status of these animals worsened. This mimics the clinical situation as patients become less responsive to the drug. The combination of ISIS 113715 and metformin produced an additive effect; furthermore, this reduction in blood glucose was durable and lasted throughout the study. ISIS 113715 alone also had a similarly sustained effect.

No hypoglycemia resulted from the combination of metformin and ISIS 113715 even at high doses. There was no significant change in AST/ALT, plasma triglyceride or plasma cholesterol levels in any animal group. Because of the risk of lactic acidosis associated with metformin treatment, plasma lactate levels were measured. A dose-dependent increase in lactate was seen after 4 weeks of treatment with metformin. Similar levels of lactate were seen in rats given a combination of metformin and ISIS 113715, indicating that the potential for lactic acidosis is not believed to be compounded by the combination treatment. ISIS 113715 alone did not cause an increase in plasma lactate.

### Example 32

### Combination Therapy Using ISIS 113715 and Sulfonylurea in ZDF Rats

The sulfonylureas, or sulphonylureas, are a class of hypoglycemic agents that enhance secretion of insulin from pancreatic beta-cells. Sulfonylureas may also cause a reduction in serum glucagon and potentiate the action of insulin at the extrapancreatic tissues. They vary in potency tremendously with first generation sulphonylureas such (e.g. tolbumatide, chlorpropamide) being less potent than second generation sulphonylureas (e.g. glipizide, glimepiride). They are given orally. The sulfonylureas can cause weight gain, and carry a risk of hypoglycemia.

A combination of ISIS 113715 and glipizide is administered to ZDF rats. Rats are given ISIS 113715 by intraperitoneal injection at 25 mg/kg twice a week and glipizide (orally administered, 10 mg/kg/day), for three weeks. Plasma glucose is measured at week 0 (before treatment) and after weeks 1, 2 and 3 of treatment.

These rats are weighed at week 2 and week 3 of treatment. AST/ALT and plasma cholesterol levels are measured in the rats at weeks 1, 2, 3 and 4.

Glucose and insulin tolerance tests (ITT) are administered to the rats (at week 3). Insulin (1.5 U/kg, in PBS @ 3 U/mL) is injected and plasma glucose is measured over time. The results are graphed and the area under the curve (AUC) is a measure of insulin sensitivity.

### Example 33

### Combination of ISIS 113715 with GLP Analogs

GLP-1 analogs are being evaluated for clinical use as antidiabetic agents. GLP-1 itself has a short half-life due to N-terminal degradation of the peptide by Dipeptidyl Peptidase (DPP-IV)-mediated cleavage at the position 2 alanine. This limits the clinical usefulness of native GLP-1 or synthetic versions thereof. Longer acting analogs have been developed, including Exendin-4 (Exenatide™, Exenatide LAR™), a DP IV-resistant GLP-1 analog and Liraglutide™, an acylated albumin-bound human GLP-1 analog.

Chronic (5 to 6 week) exendin-4 administration to ZDF rats has been shown to be associated with a reduction in glycated hemoglobin compared with saline treatment. In hyperinsulinemic-euglycemic clamp studies performed after the last injection, rats treated with exendin-4 showed a 50% improvement in insulin sensitivity (Young et al., 1999, Diabetes 48, 1026-1034).

A combination of ISIS 113715 and a GLP-1 analog is administered to aged ZDF rats. Rats are given ISIS 113715 by intraperitoneal injection at 25 mg/kg twice a week and Exendin-4 (intraperitoneal injection, 0.2 μg/kg twice daily), for three weeks. Plasma glucose is measured at week 0 (before treatment) and after weeks 1, 2 and 3 of treatment.

These rats are weighed at week 2 and week 3 of treatment. AST/ALT and plasma cholesterol levels are measured in the rats at weeks 1, 2, 3 and 4.

Glucose and insulin tolerance tests (ITT) are administered to the rats at week 3. Insulin is injected and plasma glucose is measured over time. The results are graphed and the area under the curve (AUC) is a measure of insulin sensitivity.

### Example 34

### Human Clinical Trials—Combination with Metformin, Glipizide, Rosiglitazone (CS-3)

A drug interaction study was performed using 30 normal human subjects (3 cohorts). ISIS 113715 (200 mg) was administered subcutaneously and each cohort received either metformin (500 mg), glipizide (5 mg) or rosiglitazone (2 mg), administered orally. Oral agent was administered on day 1 and day 8, and ISIS 113715 (or placebo) was administered on day 4, day 6 and day 8. Not all subjects received all 3 injections. The endpoints of the study were safety and tolerability and pharmacokinetic analysis. By completion of study, no pharmacokinetic interactions were observed for either ISIS 113715 or the co-administered oral anti-diabetic drugs. ISIS 113715 was found to be safe and well tolerated when administered subcutaneously to normal volunteers. Some local erythema and induration was noted at the injection site; no systemic effects were observed.

### Example 35

### Human Clinical Trials—Phase II Subcutaneous ISIS 113715 in Combination with Sulfonylurea (CS-4)

A Phase II study is conducted on 75 Type 2 diabetics (5 cohorts). Patients are given ISIS 113715 at 50, 100, 200 or 400 mg per week, along with 5 mg sulfonylurea (Glipizide/Glyburide). ISIS 113715 is administered subcutaneously (SC). Week 1 is the loading period (IV doses at 50, 100, 200 or 400 mg per week (divided into 3 doses given in a one-hour infusion on days 1, 3 and 5), then drug is given daily SC for 5 weeks. The sulfonylurea is given orally once daily (weekly dose divided by 7) for 5 weeks. The study is a 13-week study (2 weeks screening, 3 weeks baseline, 6 weeks treatment, 4 weeks follow up). Endpoints are safety and tolerability, pharmacokinetics, IVGTT (glucose, insulin, C-peptide, fasted blood sugar). An open-label extension (CS-5) of this study uses a safe and efficacious dose of ISIS 113715 (determined in CS-4) in combination with 5 mg glipizide/glyburide, dosed as in CS-4 for up to 13 weeks.

### Example 36

### Human Clinical Trials—Phase II Subcutaneous ISIS 113715 in Combination with Metformin (CS-6)

A Phase II study is conducted on 75 Type 2 diabetics (5 cohorts). Patients are given ISIS 113715 at 50, 100, 200 or 400 mg, along with 500 mg Metformin. ISIS 113715 is administered subcutaneously. Week 1 is the loading period (as in previous example), then drug is given daily SC for 5 weeks. The metformin is given orally once daily for 6 weeks. The study is a 13-week study (2 weeks screening, 3 weeks baseline, 6 weeks treatment, 4 weeks follow up). Endpoints are safety and tolerability, pharmacokinetics, IVGTT (glucose, insulin, C-peptide, FBS).

### Example 37

### Specificity of PTP1B Inhibition by ISIS 113715

There is a high degree of homology between PTP1B and T-cell phosphatase (TC-PTPase, PTPN2), particularly in the catalytic domain. This may present problems in design of small molecule drugs specific for PTP1B. The ability of ISIS 113715 to reduce levels of PTP1B specifically (i.e., and not reduce levels of TC-PTPase) was tested. HEPG2 human hepatocellular liver carcinoma cells (ATCC, Manassas Va.) are routinely maintained in minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum.

Cells were treated with 150 nM ISIS 113715 as in previous examples. Levels of PTP1B and TC-PTPase protein were measured by Western blot using mouse monoclonal antibodies to PTP1B (AB-1) and TC-PTPase (AB-1) (CalBiochem/Oncogene sciences, EMD Biosciences, Inc., San Diego Calif.), which specifically identified human target proteins and were used at 0.25 ug/ml. Results were expressed as percent of control (no oligo treatment).

ISIS 113715 reduced PTP1B protein levels by 89% and reduced TC-PTPase levels by 5%. A negative control oligonucleotide (ISIS 141923) reduced neither PTP1B nor TC-PTPase levels. This demonstrates that antisense inhibition of PTP1B by ISIS 113715 is both potent and specific.

### Example 38

### Sustained Effects of ISIS 113715 in ob/ob Mice After a Loading/Maintenance Regimen

Ob/ob mice were dosed weekly for four weeks using a combined loading and maintenance dose protocol. Two such protocols (high and low dose) were evaluated. In the high dose protocol, mice received a single IP injection of 50 mg/kg ISIS 113715 in the first week and a single IP injection of 20 mg/kg in each of the second, third and fourth weeks. Blood glucose was measured weekly through week 8 (four weeks after cessation of treatment). In the low dose protocol, mice received a single IP injection of 20 mg/kg ISIS 113715 in the first week and a single IP injection of 10 mg/kg in each of the second, third and fourth weeks. Blood glucose was measured weekly through week 8 (four weeks after cessation of treatment). Results are shown in Table 19.

TABLE 19
Blood glucose levels (mg/dL) are reduced for 4 wk following
a loading/maintenance regimen of ISIS 113715 treatment

| | Blood glucose levels (mg/dL) | | | | | | | | |
|---|---|---|---|---|---|---|---|---|---|
| | Week 0 | Week 1 | Week 2 | Week 3 | Week 4 | Week 5 | Week 6 | Week 7 | Week 8 |
| 113715 | 233 | 164 | 140 | 116 | 149 | 125 | 130 | 167 | 188 |
| high dose | | | | | | | | | |
| 113715 | 213 | 191 | 131 | 131 | 140 | 125 | 130 | 158 | 137 |
| low dose | | | | | | | | | |
| saline | 255 | 224 | 239 | 235 | 245 | 209 | 221 | 277 | 263 |

As shown in Table 19, blood glucose levels remain below baseline values even four weeks after cessation of weekly dosing. Concentration of ISIS 113715 in liver was measured at cessation of dosing (4 week timepoint) and was found to be 57.6±2.0 μg/g for the high dose regimen and 35.9±3.6 μg/g for the low dose regimen.

### Example 39

### Supply of ISIS 113715 in Vials

ISIS 113715 is provided as a 10 mg/mL, 200 mg/mL, or 250 mg/mL sterile solution in stoppered and sealed glass vials. The 10 mg/mL ISIS 113715 formulation is isotonic and contains phosphate buffer and sodium chloride in Water for Injection (WFI). The 200 mg/mL and 250 mg/mL formulations are hypertonic and contain only ISIS 113715 in WFI. These drug products are for single use and contain no preservatives.

ISIS 113715 injection may also be supplied as sterile 150 mg/vial lyophilized powder contained in stoppered glass vials. Sterile preserved diluent containing 0.3% metacresol is also supplied to reconstitute the lyophilized drug.

### Example 40

### Pharmacokinetic Analysis for Clinical Studies

Non-compartmental pharmacokinetic analysis of ISIS 113715 will be carried out on each individual patient data set. The maximum observed drug concentration (Cₘₐₓ) and the time taken to reach Cₘₐₓ (Tₘₐₓ) will be obtained directly from the concentration-time data. The plasma disposition half-life (t_{1/2λz}) associated with the apparent terminal elimination phase will be calculated from the equation, t_{1/2λz}=0.693/λ_{z}, where λ_{z} is the rate constant associated with the apparent terminal elimination phase. Following single and multiple dosing, area under the plasma concentration-time curve from zero time (pre-dose) to infinite time (AUC_{∞}) will also be calculated using the linear trapezoidal rule. Area under the plasma concentration-time curve from zero time (pre-dose) to infinite time (AUC_{∞}) will also be calculated using the linear trapezoidal rule and extrapolation to infinity by dividing the final measurable concentration (Cₗₐₛₜ) by λ_{z}. Further, partial areas under the plasma concentration-time curve from zero time (pre-dose) to selected times (t) after the start of the i.v. infusion (AUCₜ) may be calculated using the linear trapezoidal rule. Plasma clearance (CL) will be calculated from CL=Actual Dose/AUC_{∞} Steady-state volume of distribution [Vₛₛ=AUMC_{∞} Nominal Dose]/(AUC_{∞})², where AUMC_{∞} is the area under the first moment curve] will also be calculated.

The amount of ISIS 113715 and total oligonucleotide excreted in the urine will be determined from the following expression:

Aet=Cᵤᵣᵢₙₑ×Vᵤᵣᵢₙₑ

where Aet is the amount excreted up to some fixed time t (i.e., 24 hours), Curine is the urine concentration of the analyte, and Vurine is the total urine volume. The percentage of the administered dose excreted in urine (intact or as total oligonucleotide) was then calculated from the following expression:

% Dose Excreted=(Aet/Administered dose)×100%

### Example 41

### Human Clinical Trials of ISIS 113715—A Randomized, Double-Blind, Placebo-Controlled, Dose-Escalation Study to Evaluate the Safety, Tolerability, Pharmacokinetics, and Activity of ISIS 113715 Administered Daily in Patients with Type 2 Diabetes Mellitus Being Treated with Sulfonylurea (CS-12)

A Phase 2 clinical study is designed to evaluate the safety, tolerability, and pharmacokinetics of two ISIS 113715 subcutaneous doses in combination with sulfonylurea (SU) versus SU and placebo. In particular, this study focuses on patients with inadequately controlled Type 2 diabetes (defined as fasting plasma glucose [FPG] of 150-270 mg/dL (8.3-14.9 mmol/L) and HbA_{1c} of 8.0-11.0%) despite ongoing treatment with sulfonylurea. One embodiment of the present invention is a method of treating a subject with inadequately controlled Type 2 diabetes comprising administering ISIS 113715. The study will also examine the effect of treatment with the two doses of ISIS 113715 in combination with SU on fasting plasma glucose and HbA_{1c} compared to treatment with SU and placebo. Also the effects of ISIS 113715 in combination with SU and SU and placebo on: insulin sensitivity and β-cell function (QUICKI and HOMA-B indices), proinsulin/insulin ratio, fasting insulin, C-peptide and proinsulin, lipids and lipoprotein values (including apoB-100), hematology, liver and renal function (including estimated GFR), blood pressure and body weight, and weekly 7-point glucose profile will be evaluated.

Patients are required to be either on the maximum treatment dose recommended for their SU or on the maximum dose the patient is able to tolerate. Further, the patient must be on a stable dose for at least 3 months prior to screening evaluations and will be required to continue their stable dose of SU (unless reduced per protocol) throughout the study. The preferred SU is glibenclamide, but glipizide is permitted. The potential enrollment of patients using other SUs must be discussed with the Isis Medical Monitor. The treatment cohorts are as follows:

Cohort A=100 mg ISIS 113715 or placebo given thrice in Week 1 by 1-hour i.v. infusion and 15 mg ISIS 113715 or placebo by daily s.c. injection during Weeks 2-7 and 9-14.
Cohort B=200 mg ISIS 113715 or placebo given thrice in Week 1 by 1-hour i.v. infusion and 15 mg ISIS 113715 or placebo by daily s.c. injection during Weeks 2-7 and 9-14.

Eighteen patients will be enrolled into Cohort A and randomized at a 2:1 ratio to receive treatment with either 15 mg/day ISIS 113715 or placebo, both in combination with a stable dose of SU. Enrollment of 18 patients into Cohort B (dose escalation) will begin once (1) 6 patients in Cohort A have completed Treatment Period 1 with a satisfactory safety profile, and (2) 18 patients have been assigned to Cohort A. Other than dosage, patients in Cohort B will follow the same schedule as Cohort A.

Diagnosis and main criteria for inclusion are male or female (post-menopausal and/or surgically sterile) aged 18 to 70 years diagnosed with type 2 diabetes mellitus of less than or equal to 8 years in duration who are being treated with SU at a stable maximum dose for less than or equal to 3 months prior to screening having fasting blood glucose levels of 150 to 270 mg/dL and HbA, levels of 8.0-11.0%

Main exclusion criteria are greater than 3 severe hypoglycemia episodes within 6 months of screen, complications of diabetes (e.g., neuropathy, nephropathy, and reginopathy), clinically significant and currently active diseases, clinically significant abnormalities in medical history, physical examination, or laboratory examination.

Loading dose of ISIS 113715 (100 or 200 mg/infusion) or placebo will be administered via a 1-hour i.v. infusion on Days 1, 3, and 5 for a total of three infusions (300 mg/week or 600 mg/week for patients randomized to receive ISIS 113715 in Cohorts A and B, respectively). During the remainder of treatment (Weeks 2 to 7 and 9 to 14), patients will self-administer s.c. injections of ISIS 113715 (15 mg or 30 mg) or placebo once-daily in the morning. All patients will continue to take their prescribed daily dose of oral SU during the dosing period unless dose reductions are required.

Pharmacokinetic profiles will be assessed in all patients receiving doses of ISIS 113715 and SU and all patients receiving doses of placebo and SU in each cohort. Pharmacologic activity will be assessed by measurement of the following: HbA_{1c} and fasting glucose, weekly seven-point glucose profile, mean fasting insulin and C-peptide, fasting proinsulin, lipid and lipoprotein values, insulin sensitivity and β-cell function, and adiponectin levels.

For the i.v. loading doses during Week 1, Investigators will receive stopper glass vials containing 1 mL sterile solution that is composed of either 200 mg/mL ISIS 113715 Injection (ISIS 113715 in Water for Injection) or placebo (Water for Injection, 0.004 mg/mL riboflavin, and 9.0 mg/mL sodium chloride). The solution drug product vials are single-use only.

For the s.c. injections during Weeks 2-7 and 9-14, the Investigator will be provided with stoppered glass vials containing sterile lyophilized powder that is composed of either 150 mg ISIS 113715 or placebo. In addition to these drug products, Investigators will also be given a diluent for reconstitution of the lyophilized drug product. The diluent is 0.3% Metacresol for Injection, which contains Water for Injection, 3.00 mg/mL metacresol, 0.26 mg/mL sodium phosphate monobasic monohydrate, and 2.14 mg/mL sodium phosphate dibasic heptahydrate.

For reconstitution of the Investigation Drug lyophilized product, 1.4 mL of 0.3% Metacresol for Injection will be added per vial of lyophilized product by a pharmacist or designee using aseptic techniques. At least 10 minutes should be allowed for dissolution of the lyophilized material into the diluent. Once reconstituted, the Investigational Drug solution should be stored at 2 to 8 C and protected from light until used.

At the time of s.c. injection, reconstituted Investigational Drug solution will be withdrawn from the vial and either 0.15 mL (Cohort A) or 0.30 mL (Cohort B) will be injected into one of four quadrants of the abdomen. The site of injection should be rotated daily.

1. A method of reducing HbA_{1c} levels in a subject, comprising administering to said subject an oligonucleotide having the nucleobase sequence “GCTCCTTCCACTGATCCTGC” (SEQ ID NO: 17) and which is targeted to PTP1B.

2. The method of claim 1 wherein said oligonucleotide is administered in a dosing regimen comprised of a plurality of doses.

3. The method of claim 1 wherein said subject has Type 2 diabetes, obesity or metabolic syndrome.

4. The method of claim 1 wherein, prior to the step of administering, said subject exhibits fasting blood glucose levels of at least 130 mg/dL, HbA_{1c} levels of at least 6%, or body mass index greater than 25 kg/m².

5. The method of claim 4 wherein said subject does not achieve normal glucose levels on a therapeutic regimen of insulin, sulfonylurea, or metformin.

6. (canceled)

7. The method of claim 1 wherein HbA_{1c} levels are reduced to about 7% or below about 7%.

8. The method of claim 2 wherein said doses are administered approximately weekly.

9. The method of claim 2 wherein said doses are administered approximately biweekly.

10. The method of claim 2 wherein said doses are administered daily.

11. The method of claim 2 wherein each dose of said plurality of doses comprises from about 0.5 to about 7.5 mg/kg of the oligonucleotide.

12. The method of claim 2 wherein each dose of said plurality of doses comprises from about 100 to about 200 mg of the oligonucleotide.

13. The method of claim 1 wherein said oligonucleotide is characterized by a ten-deoxynucleotide gap region flanked on its 3′ and 5′ ends with five 2′-O-(2-methoxyethyl) nucleotides, and wherein all cytosines are 5-methylcytosines each internucleoside linkage is a phosphorothioate linkage.

14.-15. (canceled)

16. A method of reducing fasting glucose levels in a subject, comprising administering to said subject an oligonucleotide having the nucleobase sequence “GCTCCTTCCACTGATCCTGC” (SEQ ID NO: 17) and which is targeted to PTP1B.

17.-18. (canceled)

19. The method of claim 16 wherein fasting plasma glucose levels are reduced by at least about 25 mg/dL.

20. The method of claim 16 wherein fasting plasma glucose levels are reduced by at least about 10 mg/dL.

21.-32. (canceled)

33. A method of increasing HDL levels or HDL:LDL ratios in a subject comprising administering to said subject a plurality of doses of an oligonucleotide having the nucleobase sequence “GCTCCTTCCACTGATCCTGC” (SEQ ID NO: 17) and which is targeted to PTP1B.

34.-40. (canceled)

41. The method of claim 1 wherein said oligonucleotide is administered during a loading period and a maintenance period.

42. The method of claim 41 wherein the loading period results in at least 70-80% steady-state levels of oligonucleotide in organs.

43. The method of claim 41 wherein the loading period comprises administering the oligonucleotide to the subject once per day for up to 10 days.

44. The method of claim 41 wherein the loading period comprises administering the oligonucleotide to the subject about once per week for about 3 weeks.

45. The method of claim 41 wherein the loading period comprises administering the oligonucleotide to the subject about twice per week for about 3 weeks.

46. The method of claim 41 wherein the oligonucleotide is delivered intravenously during the loading period.

47. The method of claim 41 wherein the oligonucleotide is delivered subcutaneously during the loading period.

48.-56. (canceled)

57. The method of claim 1 further comprising administration of another glucose-lowering drug.

58. The method of claim 57 wherein said glucose-lowering drug is a PPAR agonist, a dipeptidyl peptidase (IV) inhibitor, a GLP-1 analog, insulin or an insulin analog, an insulin secretagogue, a SGLT2 inhibitor, a human amylin analog, a biguanide, or an alpha-glucosidase inhibitor.

59.-105. (canceled)

106. The method of claim 1, wherein the administering results in decreased adiposity.

107. The method of claim 1, wherein the administering results in reduced lipid levels.

108. The method of claim 107, wherein the lipid levels are triglyceride levels, cholesterol levels or a combination thereof.

109. The method of claim 108, wherein the cholesterol levels are LDL cholesterol levels, VLDL cholesterol levels or a combination thereof.


SEQUENCE LISTING (21 sequences)

SEQ ID NO: 1 - DNA, 20 bp, Artificial Sequence
  Oligomeric compound
tccgtcatcgctcctcaggg

SEQ ID NO: 2 - DNA, 20 bp, Artificial Sequence
  Oligomeric compound
atgcattctgcccccaagga

SEQ ID NO: 3 - DNA, 3247 bp, Homo sapiens
  CDS (91)...(1398)
gggcgggcctcggggctaagagcgcgacgcctagagcggcagacggcgcagtgggccgagaaggaggcgcagcagccgccctggcccgtcatggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcctgctgatggacaagaggaaagacccttcttccgttgatatcaagaaagtgctgttagaaatgaggaagtttcggatggggttgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagcctgaccctcctccactccacctccacccactgtccgcctctgcccgcagagcccacgcccgactagcaggcatgccgcggtaggtaagggccgccggaccgcgtagagagccgggccccggacggacgttggttctgcactaaaacccatcttccccggatgtgtgtctcacccctcatccttttactttttgccccttccactttgagtaccaaatccacaagccattttttgaggagagtgaaagagagtaccatgctggcggcgcagagggaaggggcctacacccgtcttggggctcgccccacccagggctccctcctggagcatcccaggcggcgcacgccaacagccccccccttgaatctgcagggagcaactctccactccatatttatttaaacaattttttccccaaaggcatccatagtgcactagcattttcttgaaccaataatgtattaaaattttttgatgtcagccttgcatcaagggctttatcaaaaagtacaataataaatcctcaggtagtactgggaatggaaggctttgccatgggcctgctgcgtcagaccagtactgggaaggaggacggttgtaagcagttgttatttagtgatattgtgggtaacgtgagaagatagaacaatgctataatatataatgaacacgtgggtatttaataagaaacatgatgtgagattactttgtcccgcttattctcctccctgttatctgctagatctagttctcaatcactgctcccccgtgtgtattagaatgcatgtaaggtcttcttgtgtcctgatgaaaaatatgtgcttgaaatgagaaactttgatctctgcttactaatgtgccccatgtccaagtccaacctgcctgtgcatgacctgatcattacatggctgtggttcctaagcctgttgctgaagtcattgtcgctcagcaatagggtgcagttttccaggaataggcatttgctaattcctggcatgacactctagtgacttcctggtgaggcccagcctgtcctggtacagcagggtcttgctgtaactcagacattccaagggtatgggaagccatattcacacctcacgctctggacatgatttagggaagcagggacaccccccgccccccacctttgggatcagcctccgccattccaagtcaacactcttcttgagcagaccgtgatttggaagagaggcacctgctggaaaccacacttcttgaaacagcctgggtgacggtcctttaggcagcctgccgccgtctctgtcccggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatatgccttaagccaatatttactcatcaggtcattattttttacaatggccatggaataaaccatttttacaaaaataaaaacaaaaaaagc

SEQ ID NO: 4 - DNA, 21 bp, Artificial Sequence
  PCR Primer
ggagttcgagcagatcgacaa

SEQ ID NO: 5 - DNA, 21 bp, Artificial Sequence
  PCR Primer
ggccactctacatgggaagtc

SEQ ID NO: 6 - DNA, 24 bp, Artificial Sequence
  PCR Probe
agctgggcggccatttaccaggat

SEQ ID NO: 7 - DNA, 19 bp, Artificial Sequence
  PCR Primer
gaaggtgaaggtcggagtc

SEQ ID NO: 8 - DNA, 20 bp, Artificial Sequence
  PCR Primer
gaagatggtgatgggatttc

SEQ ID NO: 9 - DNA, 20 bp, Artificial Sequence
  PCR Probe
caagcttcccgttctcagcc

SEQ ID NO: 10 - DNA, 4127 bp, Rattus norvegicus
  CDS (120)...(1418)
agccgctgctggggaggttggggctgaggtggtggcgggcgacgggcctcgagacgcggagcgacgcggcctagcgcggcggacggccgagggaactcgggcagtcgtcccgtcccgccatggaaatggagaaggaattcgagcagatcgataaggctgggaactgggcggctatttaccaggatattcgacatgaagccagtgacttcccatgcagaatagcgaaacttcctaagaacaaaaaccggaacaggtaccgagatgtcagcccttttgaccacagtcggattaaattgcatcaggaagataatgactatatcaatgccagcttgataaaaatggaggaagcccagaggagctatatcctcacccagggccctttaccaaacacgtgcgggcacttctgggagatggtgtgggagcagaagagcaggggcgtggtcatgctcaaccgcatcatggagaaaggctcgttaaaatgtgcccagtattggccacagaaagaagaaaaagagatggtcttcgatgacaccaatttgaagctgacactgatctctgaagatgtcaagtcatattacacagtacggcagttggagttggagaacctggctacccaggaggctcgagagatcctgcatttccactacaccacctggcctgactttggagtccctgagtcacctgcctctttcctcaatttcctattcaaagtccgagagtcaggctcactcagcccagagcacggccccattgtggtccactgcagtgctggcattggcaggtcagggaccttctgcctggctgacacctgcctcttactgatggacaagaggaaagacccgtcctctgtggacatcaagaaagtgctgttggagatgcgcaggttccgcatggggctcatccagacggccgaccaactgcgcttctcctacctggctgtgatcgagggtgcaaagttcatcatgggcgactcgtcagtgcaggatcagtggaaggagctttcccatgaagacctggagcctccccctgagcacgtgcccccacctccccggccacccaaacgcacattggagcctcacaatggcaagtgcaaggagctcttctccaaccaccagtgggtgagcgaggagagctgtgaggatgaggacatcctggccagagaggaaagcagagccccctcaattgctgtgcacagcatgagcagtatgagtcaagacactgaagttaggaaacggatggtgggtggaggtcttcaaagtgctcaggcatctgtccccactgaggaagagctgtccccaaccgaggaggaacaaaaggcacacaggccagttcactggaagcccttcctggtcaacgtgtgcatggccacggccctggcgactggcgcgtacctctgttaccgggtatgttttcactgacagactgctgtgaggcatgagcgtggtgggcgctgccactgcccaggttaggatttggtctgcggcgtctaacctggtgtagaagaaacaacagcttacaagcctgtggtggaactggaagggccagccccaggaggggcatctgtgcactgggctttgaaggagcccctggtcccaagaacagagtctaatctcagggccttaacctgttcaggagaagtagaggaaatgccaaatactcttcttgctctcacctcactcctcccctttctctggttcgtttgtttttggaaaaaaaaaaaaaagaattacaacacattgttgtttttaacatttataaaggcaggtttttgttatttttagagaaaacaaaagatgctaggcactggtgagattctcttgtgccctttggcatgtgatcagattcacgatttacgtttatttccgggggagggtcccacctgtcaggactgtaaagttcctgctggcttggtcagcccccccacccccccaccccgagcttgcaggtgccctgctgtgaggagagcagcagcagaggctgcccctggacagaagcccagctctgcttccctcaggtgtccctgcgtttccatcctccttctttgtgaccgccatcttgcagatgacccagtcctcagcaccccacccctgcagatgggtttctccgagggcctgcctcagggtcatcagaggttggctgccagcttagagctggggcttccatttgattggaaagtcattactattctatgtagaagccactccactgaggtgtaaagcaagactcataaaggaggagccttggtgtcatggaagtcactccgcgcgcaggacctgtaacaacctctgaaacactcagtcctgctgcagtgacgtccttgaaggcatcagacagatgatttgcagactgccaagacttgtcctgagccgtgatttttagagtctggactcatgaaacaccgccgagcgcttactgtgcagcctctgatgctggttggctgaggctgcggggaggtggacactgtgggtgcatccagtgcagttgcttttgtgcagttgggtccagcagcacagcccgcactccagcctcagctgcaggccacagtggccatggaggccgccagagcgagctggggtggatgcttgttcacttggagcagccttcccaggacgtgcagctcccttcctgctttgtccttctgcttccttccctggagtagcaagcccacgagcaatcgtgaggggtgtgagggagctgcagaggcatcagagtggcctgcagcggcgtgaggccccttcccctccgacacccccctccagaggagccgctccactgttatttattcactttgcccacagacacccctgagtgagcacaccctgaaactgaccgtgtaaggtgtcagcctgcacccaggaccgtcaggtgcagcaccgggtcagtcctagggttgaggtaggactgacacagccactgtgtggctggtgctggggcaggggcaggagctgagggtcttagaagcaatcttcaggaacagacaacagtggtgacatgtaaagtccctgtggctactgatgacatgtgtaggatgaaggctggcctttctcccatgactttctagatcccgttccccgtctgctttccctgtgagttagaaaacacacaggctcctgtcctggtggtgccgtgtgcttgacatgggaaacttagatgcctgctcactggcgggcacctcggcatcgccaccactcagagtgagagcagtgctgtccagtgccgaggccgcctgactcccggcaggactcttcaggctctggcctgccccagcacaccccgctggatctcagacattccacacccacacctcattccctggacacttgggcaagcaggcccgcccttccacctctggggtcagcccctccattccgagttcacactgctctggagcaggccaggaccggaagcaaggcagctggtgaggagcaccctcctgggaacagtgtaggtgacagtcctgagagtcagcttgctagcgctgctggcaccagtcaccttgctcagaagtgtgtggctcttgaggctgaagagactgatgatggtgctcatgactcttctgtgaggggaacttgaccttcacattgggtggctttttttaaaataagcgaaggcagctggaactccagtctgcctcttgccagcacttcacattttgcctttcacccagagaagccagcacagagccactggggaaggcgatggccttgcctgcacaggctgaggagatggctcagccggcgtccaggctgtgtctggagcagggggtgcacagcagcctcacaggtgggggcctcagagcaggcgctgccctgtcccctgccccgctggaggcagcaaagctgctgcatgccttaagtcaatacttactcagcagggcgctctcgttctctctctctctctctctctctctctctctctctctctctctctctctaaatggccatagaataaaccattttacaaaaataaaagccaacaacaaagtgctctggaatagcacctttgcaggagcggggggtgtctcagggtcttctgtgacctcaccgaactgtccgactgcaccgtttccaacttgtgtctcactaatgggtctgcattagttgcaacaataaatgtttttaaagaac

SEQ ID NO: 11 - DNA, 21 bp, Artificial Sequence
  PCR Primer
cgagggtgcaaagttcatcat

SEQ ID NO: 12 - DNA, 21 bp, Artificial Sequence
  PCR Primer
ccaggtcttcatgggaaagct

SEQ ID NO: 13 - DNA, 26 bp, Artificial  Sequence
  PCR Probe
cgactcgtcagtgcaggatcagtgga

SEQ ID NO: 14 - DNA, 23 bp, Artificial Sequence
  PCR Primer
tgttctagagacagccgcatctt

SEQ ID NO: 15 - DNA, 21 bp, Artificial Sequence
  PCR Primer
caccgaccttcaccatcttgt

SEQ ID NO: 16 - DNA, 24 bp, Artificial Sequence
  PCR Probe
ttgtgcagtgccagcctcgtctca

SEQ ID NO: 17 - DNA, 20 bp, Artificial Sequence
  Oligomeric compound
gctccttccactgatcctgc

SEQ ID NO: 18 - DNA, 2346 bp, M. musculus
gaattcgggatccttttgcacattcctagttagcagtgcatactcatcagactggagatgtttaatgacatcagggaaccaaacggacaacccatagtacccgaagacagggtgaaccagacaatcgtaagcttgatggtgttttccctgactgggtagttgaagcatctcatgaatgtcagccaaattccgtacagttcggtgcggatccgaacgaaacacctcctgtaccaggttcccgtgtcgctctcaatttcaatcagctcatctatttgtttgggagtcttgattttatttaccgtgaagaccttctctggctggccccgggctctcatgttggtgtcatgaattaacttcagaatcatccaggcttcatcatgttttcccacctccagcaagaaccgagggctttctggcatgaaggtgagagccaccacagaggagacgcatgggagcgcacagacgatgacgaagacgcgccacgtgtggaactggtaggctgaacccatgctgaagctccacccgtagtggggaatgatggcccaggcatggcggaggctagatgccgccaatcatccagaacatgcagaagccgctgctggggagcttggggctgcggtggtggcgggtgacgggcttcgggacgcggagcgacgcggcctagcgcggcggacggccgtgggaactcgggcagccgacccgtcccgccatggagatggagaaggagttcgaggagatcgacaaggctgggaactgggcggctatttaccaggacattcgacatgaagccagcgacttcccatgcaaagtcgcgaagcttcctaagaacaaaaaccggaacaggtaccgagatgtcagcccttttgaccacagtcggattaaattgcaccaggaagataatgactatatcaatgccagcttgataaaaatggaagaagcccagaggagctatattctcacccagggccctttaccaaacacatgtgggcacttctgggagatggtgtgggagcagaagagcaggggcgtggtcatgctcaaccgcatcatggagaaaggctcgttaaaatgtgcccagtattggccacagcaagaagaaaaggagatggtctttgatgacacaggtttgaagttgacactaatctctgaagatgtcaagtcatattacacagtacgacagttggagttggaaaacctgactaccaaggagactcgagagatcctgcatttccactacaccacatggcctgactttggagtccccgagtcaccggcttctttcctcaatttccttttcaaagtccgagagtcaggctcactcagcctggagcatggccccattgtggtccactgcagcgccggcatcgggaggtcagggaccttctgtctggctgacacctgcctcttactgatggacaagaggaaagacccatcttccgtggacatcaagaaagtactgctggagatgcgcaggttccgcatggggctcatccagactgccgaccagctgcgcttctcctacctggctgtcatcgagggcgccaagttcatcatgggcgactcgtcagtgcaggatcagtggaaggagctctcccgggaggatctagaccttccacccgagcacgtgcccccacctccccggccacccaaacgcacactggagcctcacaacgggaagtgcaaggagctcttctccagccaccagtgggtgagcgaggagacctgtggggatgaagacagcctggccagagaggaaggcagagcccagtcaagtgccatgcacagcgtgagcagcatgagtccagacactgaagttaggagacggatggtgggtggaggtcttcaaagtgctcaggcgtctgtccccaccgaggaagagctgtcctccactgaggaggaacacaaggcacattggccaagtcactggaagcccttcctggtcaatgtgtgcatggccacgctcctggccaccggcgcgtacttgtgctaccgggtgtgttttcactgacagactgggaggcactgccactgcccagcttaggatgcggtctgcggcgtctgacctggtgtagagggaacaacaactcgcaagcctgctctggaactggaagggcctgccccaggagggtattagtgcactgggctttgaaggagcccctggtcccacgaacagagtctaatctcagggccttaacctgttcaggagaagtagaggaaatgccaaatactcttcttgctctcacctcactcctcccctttctctgattcatttgtttttggaaaaaaaaaaaaaaagaattacaacacattgttgtttttaacatttataaaggcaggcccgaattc

SEQ ID NO: 19 - DNA, 20 bp, Artificial Sequence
  Oligomeric compound
nnnnnnnnnnnnnnnnnnnn

SEQ ID NO: 20 - DNA, 20 bp, Artificial Sequence
  Oligomeric compound
ccttccctgaaggttcctcc

SEQ ID NO: 21 - DNA, 20 bp, Artificial Sequence
  Oligomeric compound
tgaacaggttaaggccctga